(19) United States
(12) Patent Application Publication
(10) Pub. No.: US 2023/0200668 A1
(43) Pub. Date: Jun. 29, 2023

(54) METHODS, SYSTEMS AND MACHINE READABLE PROGRAMS FOR CUFFLESS BLOOD PRESSURE MEASUREMENT

(71) Applicant: Digitough Health LLC, Valhalla, NY (US)

(72) Inventors: Jeffrey Reynolds, Valhalla, NY (US); Douglas Sparks, Valhalla, NY (US); Harsha Agashe, Valhalla, NY (US)

(21) Appl. No.: 18/055,884

(22) Filed: Nov. 16, 2022

Related U.S. Application Data
(63) Continuation of application No. PCT/US2021/033787, filed on May 21, 2021.
(60) Provisional application No. 63/117,761, filed on Nov. 24, 2020.
(60) Provisional application No. 63/028,453, filed on May 21, 2020.

(51) Int. Cl. A61B 5/022; A61B 5/024

(52) U.S. Cl. CPC A61B 5/02225; A61B 5/02416; A61B 2562/0247

(57) ABSTRACT

Methods, devices and machine readable programs are presented for measuring blood pressure using a device configured to guide a user regarding placement of an anatomical region of interest in relation to a sensor. The methods can include measuring, by the sensor, a pressure applied to the sensor and measuring, by the sensor, blood volume oscillations in the anatomical region of interest while pressure is being applied to the sensor by the anatomical region of interest. The methods can further include estimating, by processing circuitry (e.g, computer processor(s), memory and the like) of the device, a blood pressure value for the user from the measured pressure and the measured blood volume oscillations.

### CROSS-REFERENCE TO RELATED APPLICATIONS

The present patent application is a continuation of and claims the benefit of priority to International Patent Application No. PCT/US2021/033787, which in turn claims the benefit of priority to U.S. Provisional Patent Application No. 63/117,761, filed Nov. 24, 2020 and U.S. Provisional Patent Application No. 63/028,453, filed May 21, 2020. The present patent application is also related to U.S. patent application Ser. No. 16/809,902, filed Mar. 5, 2020. Each of the foregoing patent applications is incorporated by reference herein in its entirety for all purposes.

### BACKGROUND OF THE DISCLOSURE

### Field

The present disclosure relates to methods and devices for cuff-less blood pressure measurement.

### Description of Related Art

Hypertension afflicts about one-fourth of the world's adult population. It is a major risk factor for stroke and heart disease and is therefore a “silent killer”. Hypertension can be treated with lifestyle changes and medication. Medical therapy is associated with a 35-40% reduction in the risk of stroke and a 15-25% reduction in the risk of heart disease. Hence, hypertension management is an archetypical example of preventive, proactive healthcare. However, the detection of high blood pressure (BP) is often missed. An estimated 20% of people with hypertension in the US do not know they have it. Further, BP in known hypertensive patients is often uncontrolled. An estimated 53% of hypertensive patients in the US do not have their BP under control.

Hypertension detection and control rates are much worse elsewhere, especially in low resource settings wherein personnel trained in BP measurement and the means for people to have their BP measured are lacking. Hypertension management is complicated by the well-known masked and white coat effects in the clinic and large BP variability amongst few measurements. In fact, ambulatory BP monitoring is now considered the gold standard for the diagnosis of high BP. Ubiquitous BP monitoring technology can improve hypertension detection by providing serial, out-of-clinic measurements in the mass population and can enhance hypertension control by providing continual feedback to the individual patient.

Several methods are available for measuring BP. However, none of these methods offers ubiquitous BP monitoring capabilities. Catheterization is the gold standard method. This method measures a BP waveform by placing a strain gauge in fluid contact with blood. However, this method is invasive. Auscultation is the standard clinical method. This method measures systolic BP (SP) and diastolic BP (DP) by occluding an artery with a cuff and detecting the Korotkoff sounds using a stethoscope and manometer during cuff deflation. The first sound indicates the initiation of turbulent flow and SP, while the fifth sound is silent and indicates the renewal of laminar flow and DP. The method is non-invasive but requires a skilled operator. Further, due to safety and ecological concerns, mercury manometers are being replaced with high maintenance aneroid manometers.

Oscillometry is the most popular non-invasive and automatic method. This method measures mean BP (MP), SP, and DP using an inflatable cuff with a sensor to record the pressure inside it. The recorded cuff pressure not only rises and falls with cuff inflation and deflation but also shows tiny oscillations indicating the pulsatile blood volume in the artery. The amplitude of these oscillations varies with the cuff pressure, as the arterial blood volume-transmural pressure relationship is nonlinear. Transmural pressure of an artery is defined as the internal pressure (i.e., BP) minus the external pressure (cuff pressure in this case). The BP values are estimated from the oscillogram (i.e., the oscillation amplitudes versus the cuff pressure) using an algorithm (e.g., fixed-ratios). However, automatic cuffs do not afford ubiquitous BP monitoring capabilities. That is, people in low resource settings may not have any access to such devices; others must go out of their way (e.g., to a pharmacy) to use these devices; and even people who own a device cannot carry and use them outside their homes.

Volume clamping is a non-invasive and automatic method used in research. This method measures a finger BP waveform by using a cuff with a photoplethysmography (PPG) sensor built-in to measure the blood volume. The blood volume at zero transmural pressure is estimated by slowly varying the cuff pressure. The cuff pressure is then continually varied to maintain this blood volume throughout the cardiac cycle via a fast servo-control system. The applied cuff pressure may thus equal BP. However, in addition to requiring a cuff, the method is prohibitively expensive.

Tonometry is another research method. This method measures a BP waveform by pressing a manometer-tipped probe on an artery. The probe must flatten or applanate the artery so that its wall tension is perpendicular to the probe. However, manual and automatic applanation have proven difficult. As a result, while the method should not require any calibration, the measured waveform has been routinely calibrated with a cuff in practice. Furthermore, the method is likewise costly. As a result, cuff-less BP monitoring technology is being widely pursued.

Much of these efforts are based on the principle of pulse transit time (PTT). PTT is the time delay for the pressure wave to travel between two arterial sites. An increase in BP causes the arteries to stiffen which, in turn, causes PTT to decline. So, PTT is often inversely correlated with BP in individual subjects. Further, PTT may be simply determined from the relative timing between proximal and distal arterial waveforms.

Hence, PTT carries the advantage of possibly offering passive BP monitoring without using a cuff. However, this approach also has major disadvantages. Firstly, PTT not only changes with BP but also smooth muscle contraction (especially when measured in small arteries) and aging and disease (especially when measured in large arteries). Smooth muscle contraction occurs acutely and thus severely limits the accuracy of the approach, whereas aging and disease are longer processes that prevent PTT from being able to track chronic changes in BP such as the common development of isolated systolic hypertension due to large artery stiffening with aging. Secondly, the required calibration of PTT in units of msec to BP in units of mmHg must either be population-based and thus error-prone or involve periodic use of a BP cuff and thus not truly cuff-less.

In sum, hypertension is a major cardiovascular risk factor that is treatable, yet high BP detection and control rates are unacceptably low. Ubiquitous BP monitoring technology can improve hypertension management, but oscillometric and other available non-invasive BP measurement devices employ an inflatable cuff and therefore do not afford such monitoring capabilities. While the PTT approach can potentially permit cuff-less and passive BP monitoring, its accuracy will be limited due to confounding physiology and the need for calibration. Hence, there is a need in the art for a ubiquitous method for reliable, cuff-less measurement of BP.

### SUMMARY OF THE DISCLOSURE

The patent application provides devices, methods, and machine readable programs provided on computer readable non-transient media to measure blood pressure including detection, processing and display circuitry. An illustrative device includes a pc board including circuitry, a measuring surface to receive a user's anatomic region of interest to detect blood pressure, a first circuit configured and arranged to detect, by a first sensor, a pressure or force applied to the measuring surface, a second circuit configured and arranged to detect, by a second sensor, measured blood volume oscillations in an anatomical region of interest while force is being applied to the sensor by the anatomical region of interest, and processing circuitry configured and arranged to estimate a blood pressure value for the user from the measured pressure or force and the measured blood volume oscillations.

In accordance with further aspects of the disclosure, the circuitry can be configured and arranged to place a visual indicator on the measuring surface of the device, wherein the visual indicator is arranged in relation to the first and second sensors. At least one of the first sensor and second sensor can be integrated into the measuring surface of the device. The device can further include a compliant deflectable mass disposed adjacent to the measuring surface of the device. The deflectable mass can include a gel material. The gel material can be covered by a membrane that at least partially defines the measuring surface. The first sensor can be at least partially embedded in the compliant deflectable mass. The first sensor can be located proximate the measuring surface. The deflectable mass can include a fluid surrounded by a bladder. The bladder can at least partially define the measuring surface.

In accordance with further aspects of the disclosure, the device can further include a housing to contain the pc board. The housing can include a shell that at least partially defines the measuring surface. The shell can define a first opening therethrough within a line of sight of the second sensor. At least one of the first sensor and second sensor can be mounted on a surface of the shell. At least one sensor can be mounted on an interior surface of the shell. The housing can define a second opening to permit a line of sight from the first opening through the second opening to a detector of a further device. The further device can include a detector of a portable electronic device. The device can further include at least one LED to direct light into the anatomical region of interest. The second sensor can be disposed on a movable platform displaceable from a first position from a second position, wherein movement from the first position to the second position is resisted by a resilient element. The resilient element can include a spring. The force required to displace the resilient element can be adjusted. The resilient element can include a bladder filled by a fluid or a gel. The contents of the bladder can be reduced by opening a valve in fluid communication with an interior volume defined by the bladder. The housing can define an opening therethrough to permit filling the housing with a fluid. If desired, the housing can surround a volume filled with an optically clear medium including gel or silicone oil. The housing can define an opening therethrough in fluid communication with a first surface of a pressure sensor, wherein a second opposing surface of the pressure sensor is in mechanical communication with the measuring surface to permit measurement of a differential pressure by the pressure sensor.

In accordance with further aspects of the disclosure, the measuring surface can define a cavity therein to accommodate tissue to permit a pooling of blood within the tissue. If desired, at least one of the sensors can include a photoplethysmography (PPG) sensor and a pressure sensor. The PPG sensor can be a reflectance-mode PPG sensor array with a surface having a surface area of about 5 mm2 to about 500 mm2. The PPG sensor can include at least one photodetector and at least one light emitting diode (LED). The PPG sensor can be an array of PPG sensors including a plurality of PPG sensors and the device can be configured and arranged to choose the sensor that yields a largest amplitude oscillogram. The pressure sensor can be a thin-filmed transducer. The pressure sensor can include a plurality of pressure sensors, and each pressure sensor can be configured and arranged to measure a corresponding applied pressure. For each of the plurality of pressure sensors, the device can be configured to measure the corresponding pressure.

In accordance with further aspects of the disclosure, a processor-readable tangible non-transient medium is provided storing a computer program for operating an arrangement of processing circuitry including a device for measuring blood pressure communicatively coupled to at least one processing circuit described anywhere herein. The computer program can include instructions configured to measure, by the sensor, a measured pressure applied to the sensor, instructions configured to measure, by the sensor, measured blood volume oscillations in the anatomical region of interest while pressure is being applied to the sensor by the anatomical region of interest, and instructions configured to estimate, by the processing circuitry of the device, a blood pressure value for the user from the measured pressure and the measured blood volume oscillations.

In accordance with further aspects of the disclosure, computer program can further include instructions configured to place a visual indicator on an exterior surface of the device, wherein the visual indicator is arranged in relation to the sensor and the sensor is integrated into the exterior surface of the device. The computer program can further include instructions configured to construct two or more oscillograms for the user from the measured pressure and the measured blood volume oscillations, wherein each oscillogram in the two or more oscillograms is constructed from measurements taken at different placements of the anatomy of interest in relation to the sensor.

37. All methods for measuring blood pressure as set forth herein.

38. All devices for measuring blood pressure as set forth herein.

39. A wearable electronic device to measure blood pressure as set forth herein.

40. The wearable electronic device according to claim 39, wherein the device is a smart watch.

41. A processor-readable tangible non-transient medium to carry out any method for measuring blood pressure as set forth herein.

It is to be understood that the following detailed description is exemplary and are intended to provide further explanation of the embodiments disclosed herein.

The accompanying drawings, which are incorporated in and constitute part of this specification, are included to illustrate and provide a further understanding of the method and system of the disclosure. Together with the description, the drawings serve to explain the principles of the disclosed embodiments.

### BRIEF DESCRIPTION OF THE DRAWINGS

FIG. 1 depicts a smartphone connected device for blood pressure monitoring in accordance with the present disclosure.

FIG. 2A is an illustrative embodiment of a clinical prototype device with a top cover installed, and FIG. 2B shows the embodiment of FIG. 2A with the top cover removed.

FIG. 3 is a block diagram illustrating the electronic and system architecture of the embodiment of FIG. 2A.

FIGS. 4A-4D illustrate preprocessed PPG and pressure waveforms obtained from the embodiment of FIG. 2A.

FIG. 4E is a block diagram for illustrative fingertip oscillometric predictions.

FIG. 5 is an isometric view of an illustrative prototype commercial device in accordance with the present disclosure.

FIGS. 6A-6B are screenshots from a prototype commercial smart phone application showing an engineering mode display and a gamified display.

FIGS. 7A-7C depict a summary of clinical trial results in accordance with the present disclosure.

FIGS. 8A-8F depict aspects of an example prediction for a normotensive subject with an illustrative algorithm.

FIGS. 9A-9F depict aspects of an example prediction for a hypertensive subject with an illustrative algorithm.

FIGS. 10A-10F depict aspects of an example prediction for a low, normotensive subject with an illustrative algorithm.

FIG. 11 illustrates an implementation to improve alignment tolerance for blood pressure measurement using multiple emitters.

FIGS. 12A-12D illustrate implementations to use a deformable surface to form a more uniform applied pressure across a non-uniform anatomy, such as a finger.

FIG. 13 is an isometric exploded view of a further illustrative device showing major components and subsystems.

FIG. 14 is a schematic view of an implementation of an integrated PPG and pressure sensor in accordance with the present disclosure.

FIGS. 15-51 describe further implementations in accordance with the present disclosure.

FIGS. 52-54 describe still further implementations in accordance with the present disclosure.

FIGS. 55A-55T set forth implementations of a smart watches or wearable devices in accordance with the present disclosure.

FIGS. 56A-61 set forth additional aspects of blood pressure measurement devices, or components thereof, in accordance with the present disclosure.

FIG. 62 illustrates aspects of a computer systemization in accordance with the present disclosure.

### DETAILED DESCRIPTION

Embodiments in accordance with the present disclosure, referred to herein at times as the Digitouch Health TouchBP™ device, can measure blood pressure at an anatomical location, such as a fingertip with a small button-like sensor by performing an oscillometric measurement on the transverse palmar arch artery in the pad of a subject's finger.

If desired, the anatomical region of interest can alternatively include, for example, one or more of a superficial artery, a non-superficial artery, a digital artery other than the transverse palmer arch artery, an artery of the hand other than the transverse palmer arch artery, an artery of the thumb, an artery of the nose, an inner artery of the nose, an outer artery of the nose, an artery of the forehead, an artery of the scalp, an artery of the ear, an artery of the toe, an artery of the wrist, the radial artery, the brachial artery, the axillary artery, the subclavian artery, the ulnar artery, the femoral artery, the popliteal artery, the tibial artery, the dorsalis pedis artery, the aorta, and the iliac artery.

In the illustrated example of the transverse palmar arch artery, the user can apply pressure with their finger against a fingertip pad. The fingertip pad can have a variety of sensors, such as a photoplethysmograph, which in turn can include one or more light emitting diodes (“LEDs”) and one or more photodiodes to facilitate measurement of blood volume oscillations, and a pressure sensor for measuring the applied pressure. As the pressure on the fingertip pad increases, the blood volume oscillations vary in amplitude, growing to a peak when the mean arterial pressure is equal to the externally applied pressure just outside the artery. An algorithm trained on a representative population analyzes the shape of the oscillogram, a curve defined by the amplitude of the oscillations, to determine the Systolic BP (SBP) and Diastolic BP (DBP).

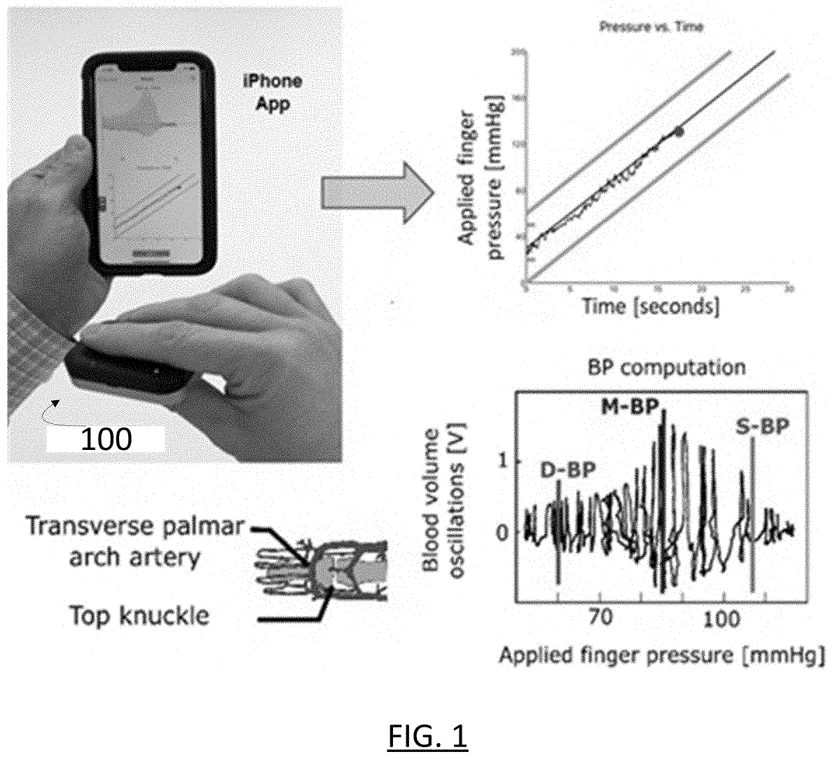

For purposes of illustration, and not limitation, as embodied herein, an implementation of such a disclosed embodiment 100 is illustrated in use in FIG. 1. Specifically, FIG. 1 illustrates a smartphone connected to a device to monitor blood pressure (BP) via an oscillometric finger pressing method. The user presses their fingertip against the device to steadily increase the external pressure on the underlying (transverse palmar arch) artery, while the device measures the applied pressure and resulting blood volume oscillations via photoplethysmography (PPG) optical signals. The phone display provides visual feedback to guide the finger actuation pressure. The device then constructs an “oscillogram” in PPG versus pressure space to compute BP.

Specially configured machine readable code within the software application displays the applied pressure to guide the user to apply a pressure ramp that is analogous to an inflating arm cuff. The user continues to apply pressure with his or her finger until the blood volume oscillations cease (in excess of the systolic pressure). The direct measurement of pressure on the artery, differentiates the disclosed approach from most other cuff-less technologies like Pulse Transit Time which are indirect measurements of pressure. Because the measurement is cuff-less and made on the fingertip, the device is small, portable, and convenient to use especially when compared to arm and wrist cuffs.

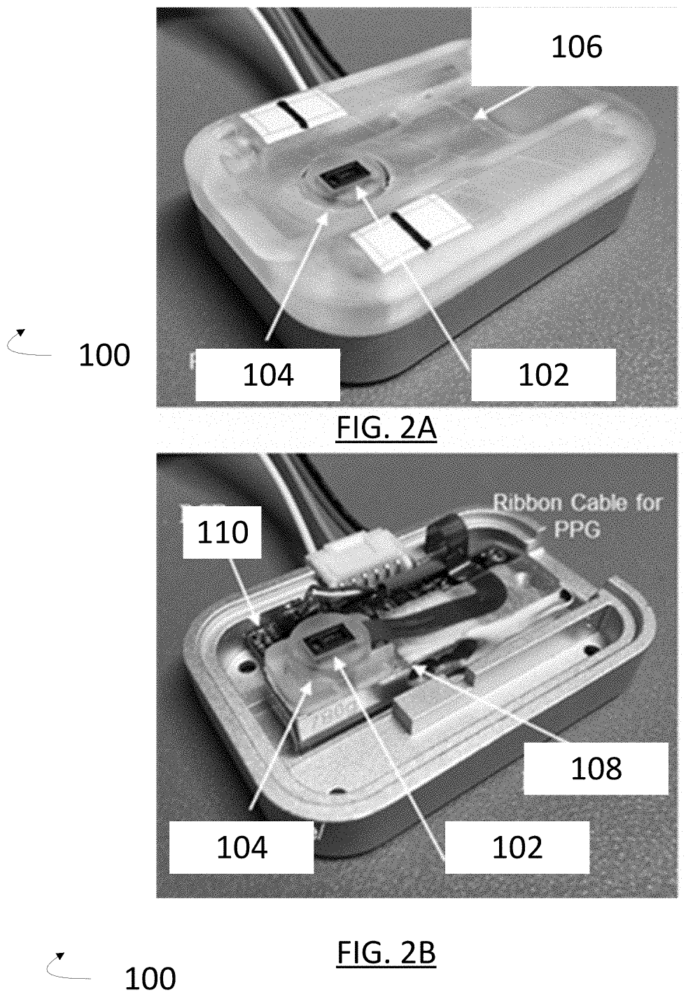

FIG. 2A is an illustrative embodiment of an illustrative prototype device 100 in accordance with the disclosure with a top cover installed, and FIG. 2B shows the embodiment of FIG. 2A with the top cover removed. FIG. 2C is a block diagram illustrating the electronic and system architecture of the embodiment of FIG. 2A. The depicted prototype sensor platform is designed to fit a handheld form factor and connect to a suitable processor to control the sensors and record measurement raw data. The sensor platform, in this example, includes a pressure sensor and an integrated PPG sensor on a chip 102, as illustrated in FIG. 2B. The illustrated pressure plate 104 is configured to hold the PPG sensor flush to the skin of a user that aligns their finger with a finger alignment groove 106 and define an area for calculating pressure from the force recorded by an underlying load cell 108. This arrangement allows for accurate clinical and laboratory assessments. But it will be appreciated by those of skill in the art that these components can be further integrated and simplified to create a commercial product. It will be appreciated that, in various embodiments, the camera of a smart phone, or other camera, can be used to photograph alignment of a finger with device 100, for example, process the image, and provide user feedback on finger alignment.

The PPG sensor system is a valuable sensor input for the oscillometric measurement and thus was given attention in the overall system design. An important component of the PPG measurement includes a light emitting diode (LED) and Photodiode Detector. The LED sends light into the highly scattering tissue which interacts with the blood pulsing through the transverse palmar arch artery in the pad of the finger, in this example. The scattered light travels back to the skin surface where it is detected by a photodiode sensor. Because the local blood volume increases at each pulse and blood has relatively higher optical absorption than the surrounding tissue, the received light signal is attenuated proportionally to the local blood volume. The strength of this absorption is a useful figure of merit in this application as well as in more traditional PPG applications, pulse rate detection and pulse oximetry.

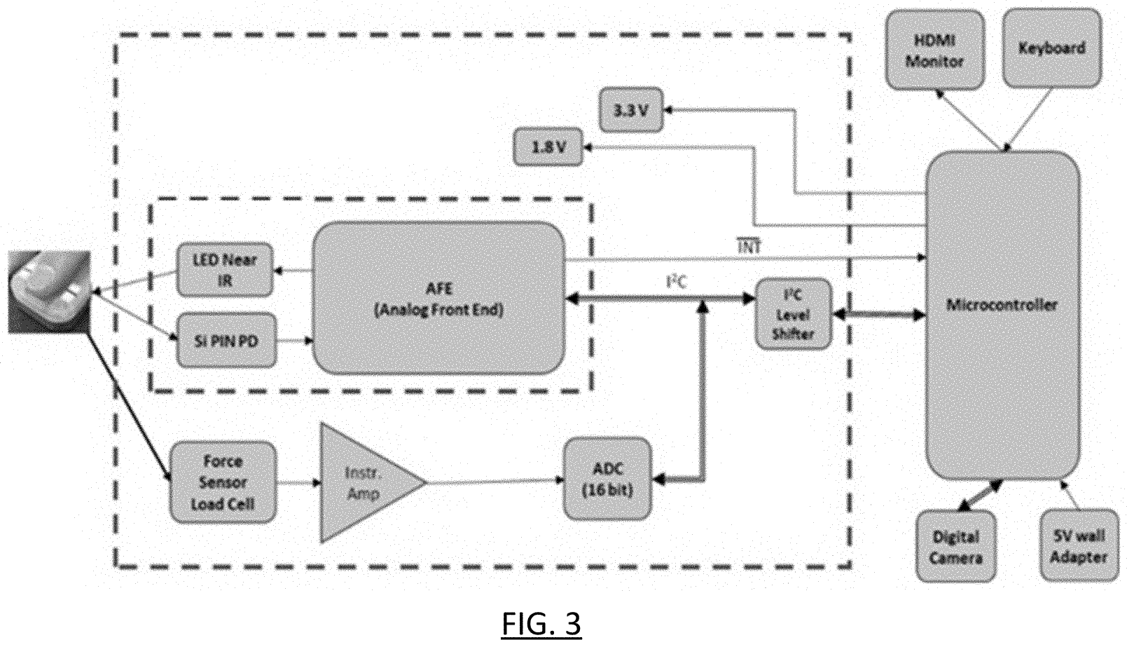

In the illustrated embodiment, and with reference to FIG. 3 in part, Applicant used an illustrative Pulse Oximeter and Heart Rate Sensor. This device can include, for example, a single chip package between 0.2 mm and about 1.5 cm on a side that can include a red and infrared LED, an integrated photodetector, preamplification, ADC, and custom signal processing, for example. The sources are pulsed and asynchronously detected with interlaced “dark” measurements between LED pulses allowing measurement of the PPG signal independent of room light interference. The device also adds analog compensation signals to the photodiode current to keep strong ambient light from saturating the PPG optical signal. This adds dynamic range to the system so that measurements can be made even in direct sunlight. The device can be controlled through an industry standard Inter-Integrated Circuit (I2C) serial bus that is standard on many low power microcontrollers. Functions that can be controlled include LED wavelength (IR and/or red), pulse amplitude, pulse frequency, and pulse duration. This provides for easy modification and optimization in both a research setting and in commercial device that can adapt to the user and environment under software (SW) control. This small circuit was integrated into a 10 mm diameter pressure pad. In some implementations, the red wavelength can be used to compute blood oxygen saturation, provide added information for the algorithm to improve BP predictions and/or detect error conditions. The pressure sensor is a further component of the sensory input for an oscillometric BP measurement. A load cell and defined pressure area sensor was selected for measuring pressure. A mechanical case and pressure plate/PPG holder were configured to integrate the PPG sensor to the load cell. The area of the pressure plate that holds the PPG sensor against the skin translates the measured force to a pressure applied to the skin. It will be appreciated by those of skill in the art that the described embodiments are illustrative only, and not intended to limit the scope of the disclosure or of the claims.

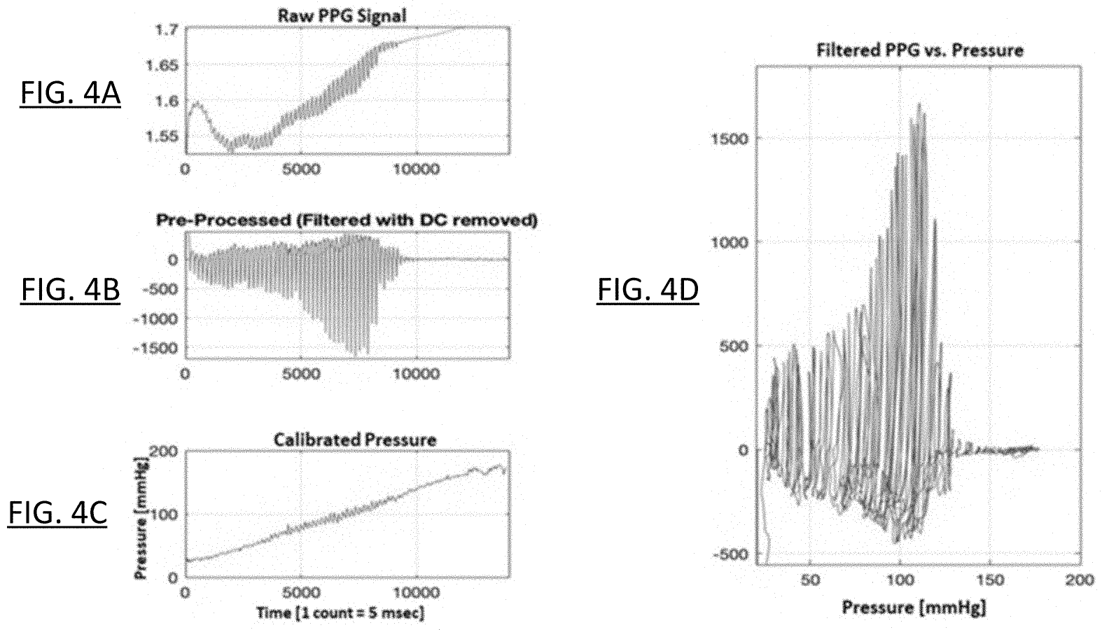

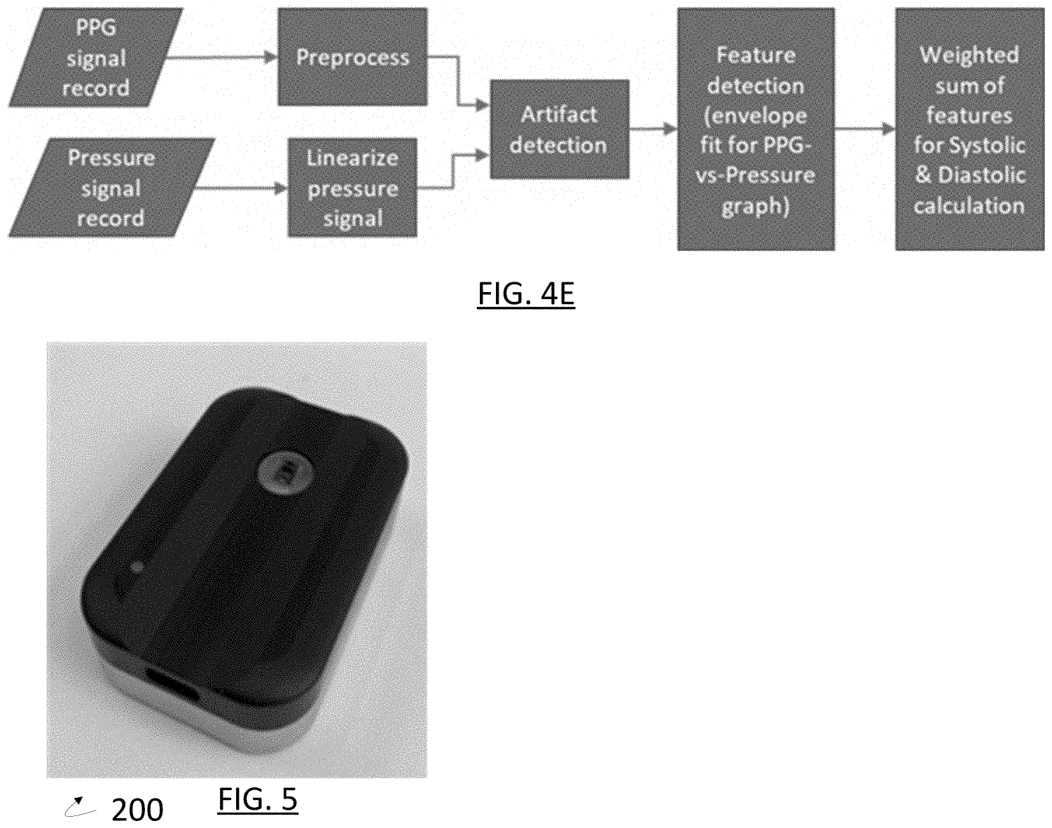

FIGS. 4A-4D illustrate preprocessed PPG and pressure waveforms obtained from the embodiment of FIG. 2A using the system and methodology illustrated in FIG. 3. FIG. 4A illustrates a raw PPG signal showing blood pulses riding on a slowly varying background, wherein the optical current is plotted in arbitrary units and the x axis samples at a rate of 200 samples/second. FIG. 4B illustrates the PPG signal with the DC or average signal removed (optical current in arbitrary units). FIG. 4C illustrates the applied pressure measured by the load cell sensor (corrected to mm-Hg). FIG. 4D illustrates the pulsatile (AC) portion of the PPG plotted in pressure space showing a classic oscillometric envelope.

FIGS. 4A-4C include the PPG and pressure readings in the time domain while the right-hand plot (FIG. 4D) combines the data into a PPG vs. pressure plot. With the system collecting high quality PPG and pressure signals, algorithms were developed to analyze the oscillometric signals to predict blood pressure.

The basic structure of the algorithm is pictured in FIG. 4E. Following preprocessing of the fingertip oscillometric signal, an algorithm fits the shape of the oscillometric envelope to the brachial reference signal. Biophysical models can be applied to help build algorithmic framework for this mapping. Various linear and nonlinear optimizations can then analyze oscillometric measurements paired with reference measurements across a large patient population to build an optimum prediction model. The raw data from the PPG and pressure are filtered to remove noise and analyzed for error conditions. They are then combined to build an oscillogram showing the blood volume versus applied pressure curve. Further automatic inspection of the oscillometric shape can further identify errors here (for example, an incomplete oscillogram). This oscillometric curve then forms the basis for prediction. Oscillogram envelope features were obtained by determining the maximum PPG excursion of the oscillogram, followed by marking the pressures at which the envelope achieved fixed percentages of the PPG maximum (e.g. pressure at which oscillogram achieves 90% of the maximum, pressure at 80% of maximum, etc). These pressure values, on each side of the oscillogram envelope, were defined as features for the next stage of the algorithm. Separate linear models were then constructed for systolic and diastolic BP values, by summing over a weighted sum of the features. The weights constitute the free parameters of the model. An algorithm's free parameters are trained on a data set of oscillometric curves with paired reference measurements by minimizing the sum of squared errors of the prediction vs. the reference. In some embodiments, a preferred alignment position is to align the midpoint of the LED and detector with the skin just proximal to the proximal end of the nail as illustrated in FIG. 3B.

The machine readable code can further include algorithms and process monitoring and error detection algorithms running in both real time and near real time. With these monitoring algorithms, the device can monitor PPG and pressure signals and guide the user to correct error conditions or reject data that is noisy. Examples of monitoring algorithms can include S/N ratio calculations, raw signal thresholds, heartbeat detection, and pulsatile modulation ratios. These preprocessing algorithms can be expanded based on the hardware modifications (for example in the case of using multiple independent LEDs as discussed elsewhere herein). By way of further example, real time signal measures can be fed back to the hardware controller to drive LED output optimization, change guidance (visual or otherwise) for the desired slope or other ramp (such as step-wise or randomized pressure levels) of the applied pressure based on heart rate or the current rate of pressure application by the user, or terminate the measurement when sufficient data has already been collected. Better and quicker feedback to the user will increase the likelihood of successful measurements and lead to higher customer satisfaction and better compliance.

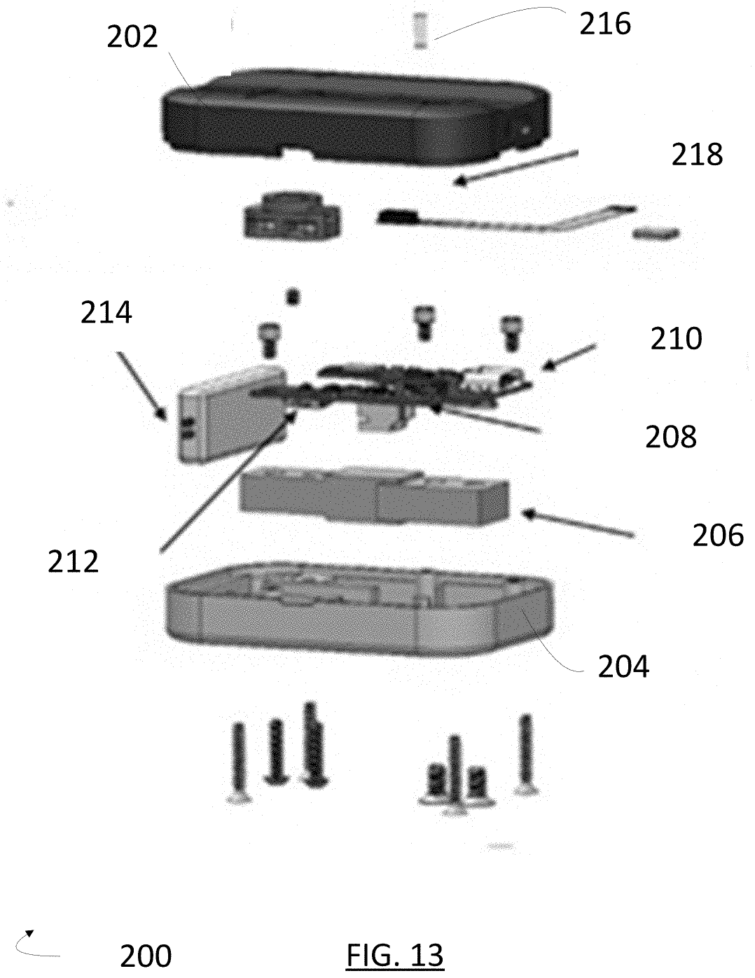

In order to demonstrate a commercial implementation of the TouchBP™ device, a commercial prototype 200 was designed, illustrated in FIG. 5. Embodiment 200 is illustrated in exploded view in FIG. 13. Embodiment 200 is defined by an upper housing portion 202 defining a finger groove therein, and a lower housing portion 204 to house system components. Load Cell 206 fits into lower housing 204, and a PC board assembly 208 including a processor and a charging port 210 and a power switch 212 are disposed over the load cell 206. The architecture is similar to the embodiment of FIG. 2A, wherein the controller includes an embedded low power BLE enabled microcontroller and the system is powered by a Lithium Polymer (LiPo) rechargeable battery 214. A light pipe or other optical conductor 216 is provided to direct light from one or more LED elements on the PC board assembly 208 to the surface of the upper housing component 202 to facilitate viewing thereof. A ribbon cable 218 is also provided to couple system components. In addition, an integrated 3-axis accelerometer was added to the embodiment of FIG. 5 in order to sense the orientation of the device 200 to improve force measurement accuracy for handheld operation, detect potential motion artifacts, and potentially provide an input for the UI. The illustrated embodiment 200 is designed to be held by the user at heart level and squeezed between the index finger and thumb or the middle finger and thumb depending on user preference. The user can also rest the device on a surface at heart level. The User Interface (UI) is provided by an App running on a smartphone in this illustration. The smartphone App communicates with the device via BLE, provides pressure feedback to the user, and records the PPG and Pressure Data. A simulated measurement is generated at the end of a pressure profile to demonstrate the complete experience. A simulated BP is used so that a BP from an unapproved device is not given to the user. Once the BP prediction is displayed it is transferred to a health tracking app, for example, to demonstrate connectivity to other health apps.

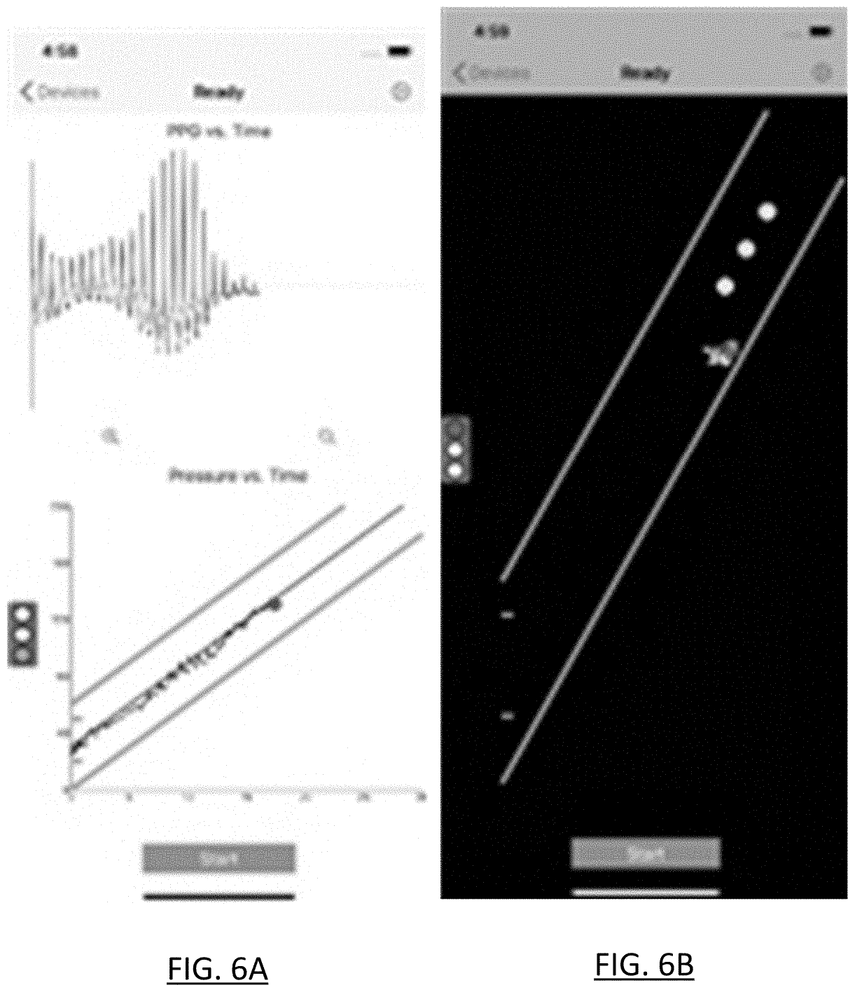

Two versions of the visual measurement guide were developed, as illustrated in FIGS. 6A and 6B: an engineering mode (FIG. 6A), which displays the change in pressure over time and the resulting PPG signal oscillation; and a gamified version (FIG. 6B) in which the user applies pressures to direct a character wearing a jet pack to collect coins. The harder the user presses, the higher the jet-pack wearing character flies. In the engineering mode the pressure signal and the filtered PPG signal are displayed. In the game mode, only the pressure feedback signal is displayed. See FIGS. 6A-6B. In the game mode, the user is guided to provide the pressure ramp needed for the measurement by guiding a jet packed character whose height is controlled by the finger pressure. The closer the user stays to the “ideal” pressure curve the more coins are collected. Alternative coin collection patterns corresponding to different pressure application means are also possible in this mode. The user can also receive a bonus when the device determines that sufficient data has been collected. Proper control of hypertension requires frequent and regular monitoring. It is believed that gamification is likely to significantly increase compliance by making the experience varied, fun, and rewarding. Thus, it will be appreciated that the system encompasses such devices, methods, and machine readable programs on non-transient media for implementing such software programs for the use of monitoring hypertension.

A clinical study was completed with 102 subjects representing a broad distribution of blood pressures covering the range required by ISO standard, 8160-2 Non-invasive sphygmomanometers Part 2: Clinical validation of automated measurement type. This standard is recognized by the FDA for clinical validation of automated blood pressure monitors. The 8160-2 standard is very prescriptive and calls for 85 subjects representing a defined range of blood pressures be compared to a reference arm cuff made using a traditional sphygmomanometer and manual auscultation. The accuracy criteria for that group of measurements is that the mean difference be less than 5 mm Hg and the standard deviation less than 8 mm Hg. For each subject a minimum of 4 Digitouch TouchBP™ readings were taken along with 4 reference arm cuff measurements made by a trained nurse. The population statistics for the study are summarized in Table 1.

TABLE 1
Study Demographics

| | | | | |
|---|---|---|---|---|
| Age Quartiles (years) | 18 | 28 | 45.5 | 81 |

| | |
|---|---|
| Mean Age (years) | 43.6 |
| Age Std. Dev. (years) | 15.9 |
| Female | 61 |
| Male | 41 |

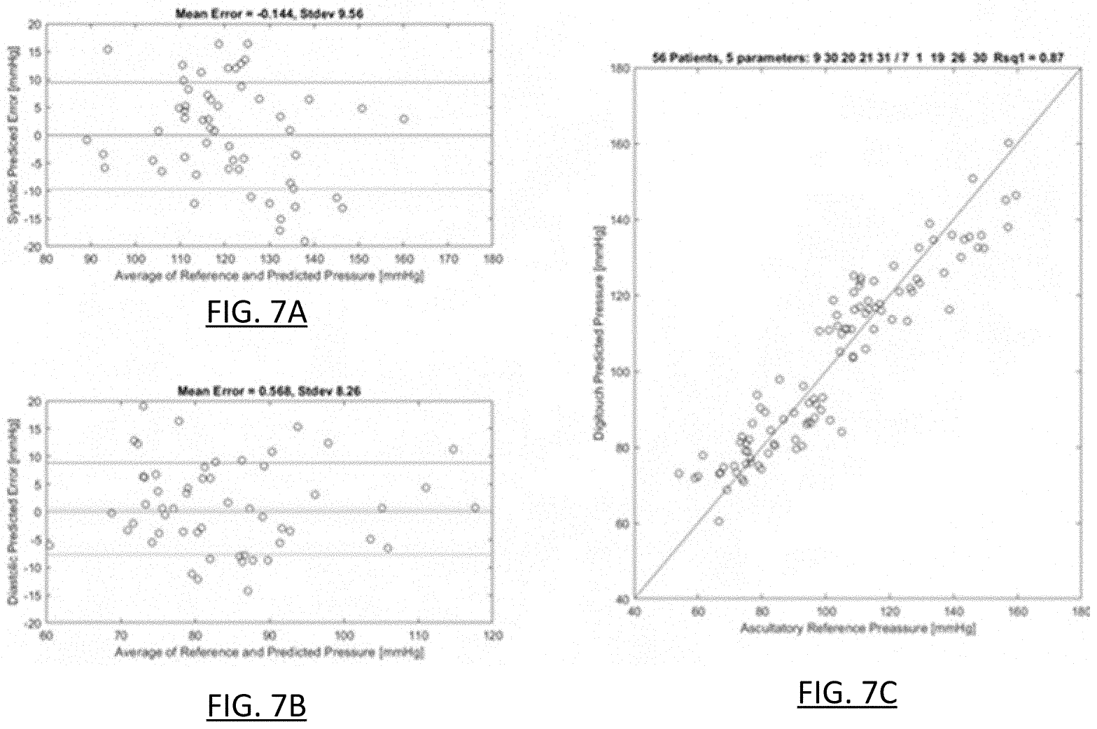

The results from the 102 subjects were split into two groups: a first group (72 subjects) and a testing group (30 subjects). The testing group was chosen to contain a representative sample of low, normal and high blood pressures. A careful review of the signals contained in the first group showed that 56 of the 72 subjects had the highest quality signals (based on a review of optimal finger-sensor alignment and the applied pressure traces). Initial results for these 56 subjects are summarized in FIGS. 7A-7C.

FIGS. 7A-7C depict a summary of results for 56 patients studied in clinical trials with paired reference readings. Reference readings were averaged per session. The plots of FIG. 7A on the left/top are Bland-Altman difference plots for Systolic Predictions vs. References and on the left/bottom are Bland-Altman difference plots for the Diastolic Predictions v. References. Horizontal lines indicate Standard Deviation and mean. On the right a standard scatter plot showing the correlation of predicted Systolic and Diastolic BPs with the Auscultatory Reference Systolic and Diastolic BPs against the blue perfect fit line with R2=0.87. These results demonstrated the successful prediction of BPs across a wide physiological range in diverse population.

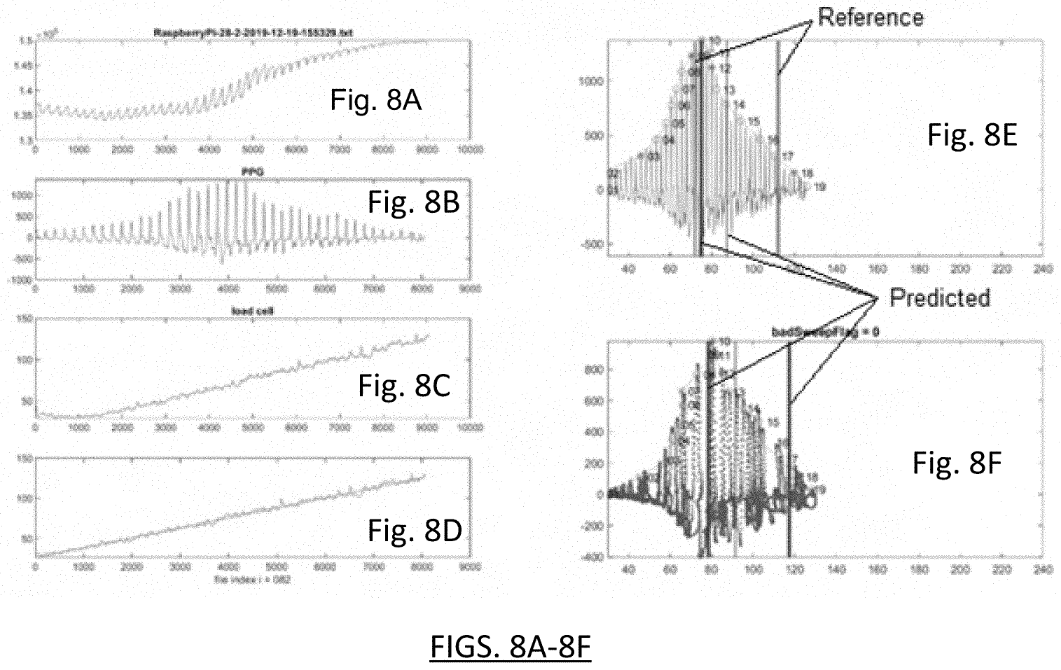

These results demonstrate the accuracy of TouchBP™ across low, normotensive, and hypertensive subjects, with DBP and SBPs ranging from 55 to 160 mm Hg. For diastolic pressure, the bias and standard deviation were 0.568 and 8.26 mm Hg, respectively. For systolic pressure, the bias and standard deviation were −0.144 and 9.56 mm Hg, respectively. In FIGS. 8A-10F a representative prediction on subjects with naturally occurring normal, hyper, and low normotensive blood pressures are displayed.

FIGS. 8A-8F depict aspects of an example prediction for a normotensive subject with an illustrative algorithm. FIGS. 8A-8D on the left depict raw and processed time domain PPG and pressure waveforms. FIG. 8E at upper right is the algorithmic fit used to build the algorithm for that measurement. Illustrated are a first set of lines that are the reference DBP and SBPs while a second set of heavy lines are the predicted values. Note that here the prediction for systolic overlaps closely with the systolic reference. FIG. 8F at bottom right is the same algorithm applied using the raw (unsmoothed) pressure signal.

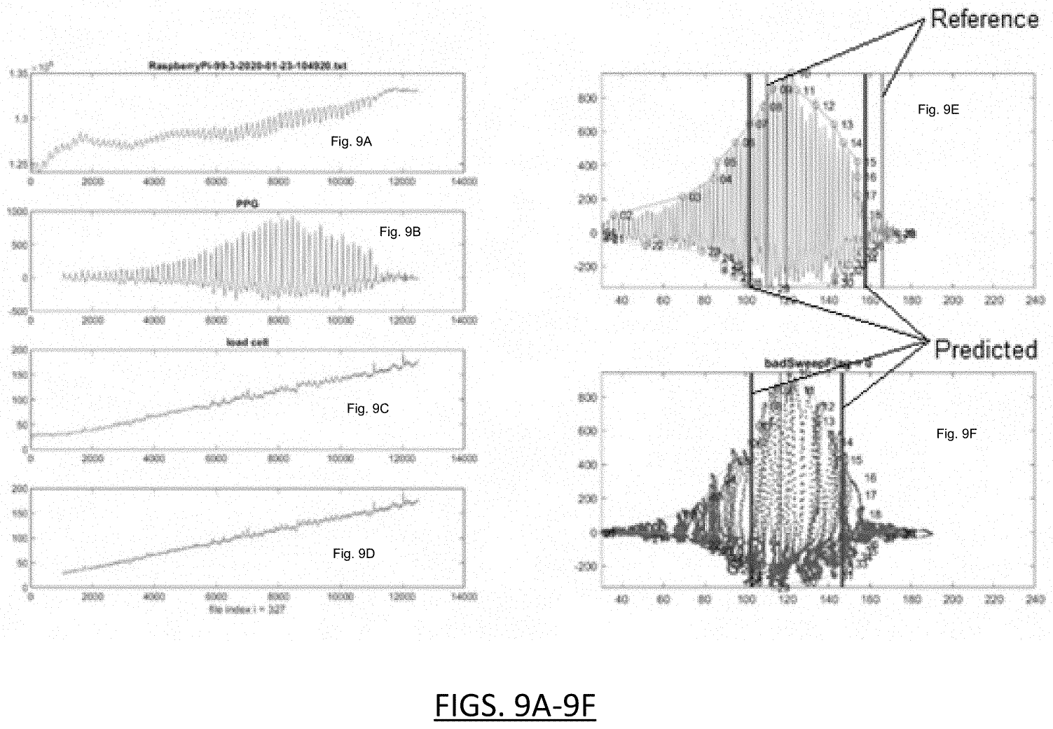

FIGS. 9A-9F depict aspects of an example prediction for a hypertensive subject with an illustrative algorithm. On the left are raw and processed time domain PPG and pressure waveforms. The upper right is the algorithmic fit used to build the algorithm for that measurement. Illustrated are a first set of reference DBP and SBPs while a second set of heavy are the predicted values. The bottom plot is the same algorithm applied using the raw pressure signal.

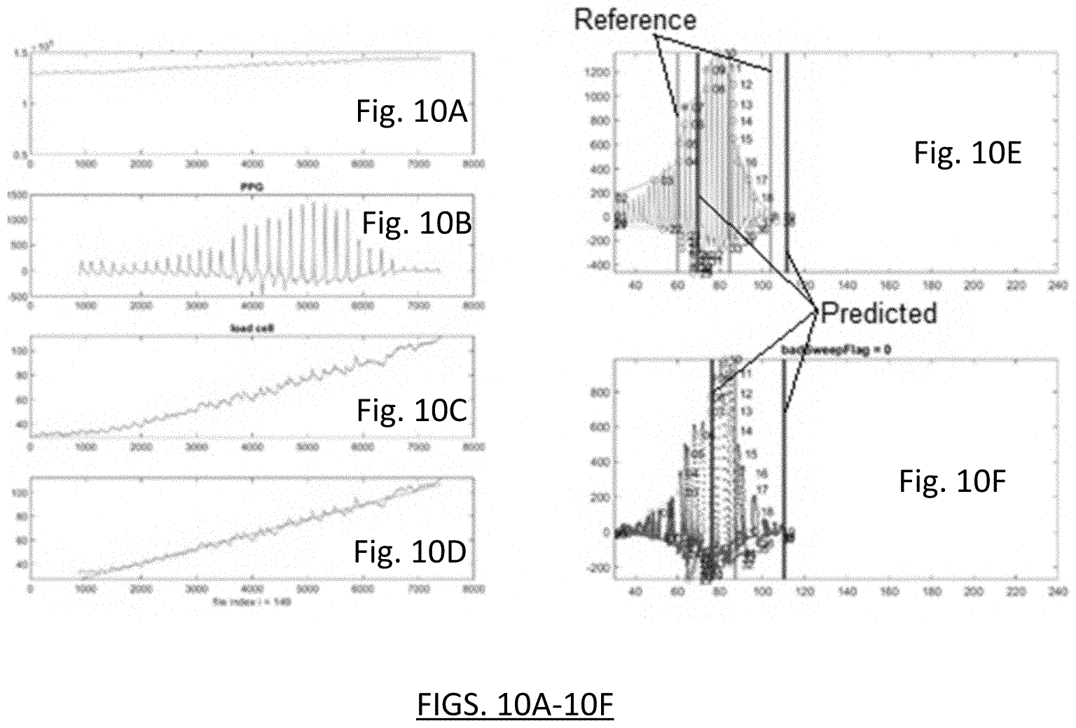

FIGS. 10A-10F depict an example prediction for a low, normotensive subject with an illustrative algorithm. On the left (FIGS. 10A-10D) are raw and processed time domain PPG and pressure waveforms. The upper right (FIG. 10E) is the algorithmic fit used to build the algorithm for that measurement. A set of vertical lines are the reference DBP and SBPs while the heavy vertical lines are the predicted values. The bottom plot (FIG. 10F) is the same algorithm applied using the raw pressure signal.

These results demonstrate that it is feasible to monitor low, normal and high blood pressures using the disclosed TouchBP™ blood pressure monitor.

Further Implementations

In further accordance with the disclosure, it is possible to provide additional implementations of blood pressure measurement devices.

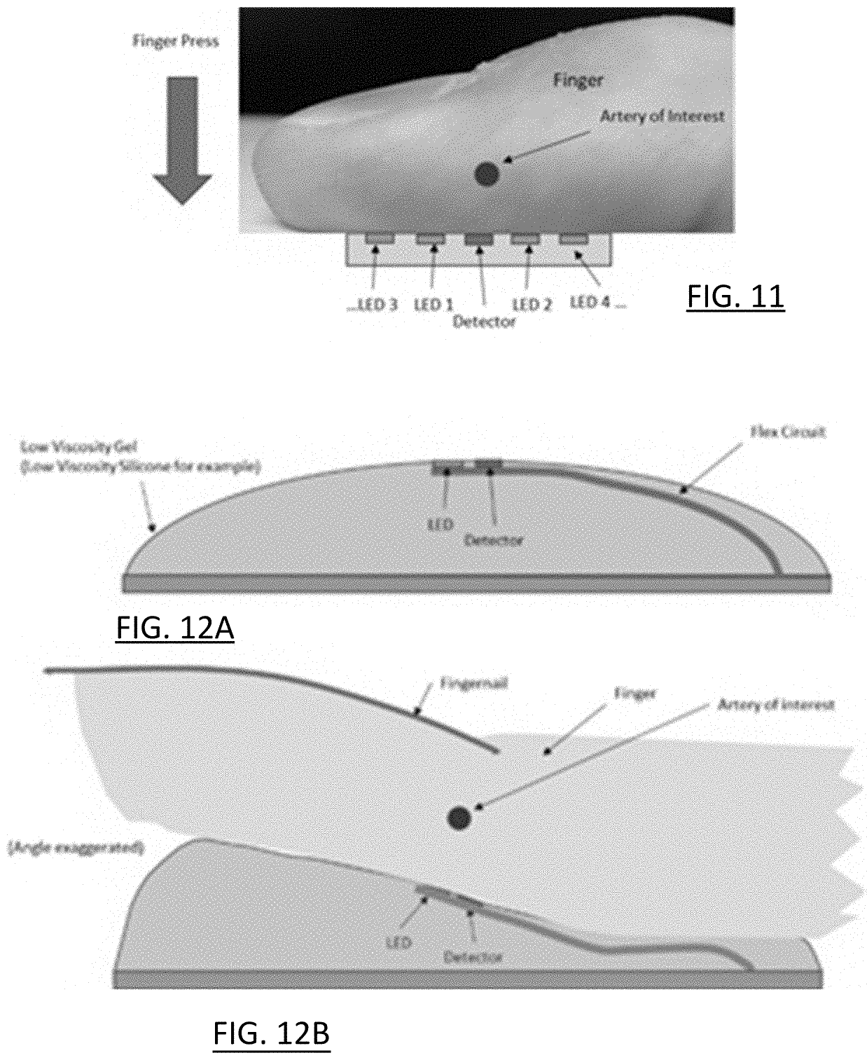

In some implementations, improved finger/sensor interfaces are provided to enhance BP measurements. Implementations to improve finger/sensor interface preferably improve both usability and accuracy of the overall system. For example, multi LED combinations can be used to automatically mitigate the effect of anatomical and alignment variations as set forth in FIG. 11. An example of this implementation is shown in FIG. 11. Each additional LED expands the range of coverage for optimizing the detection of the pulsatile component of the arterial blood volume. This can be implemented in various combinations of LEDs and detectors, and the system can, for example, monitor the multiple channels and pick the signal that has the strongest pulsatile component or other waveform or waveshape characteristics that are desirable for enhanced accuracy. The relative signal strengths can be used together, as desired, or the strongest signal can be selected for measurement. The relative signals can give an indication of finger movement that can interfere with the measurement.

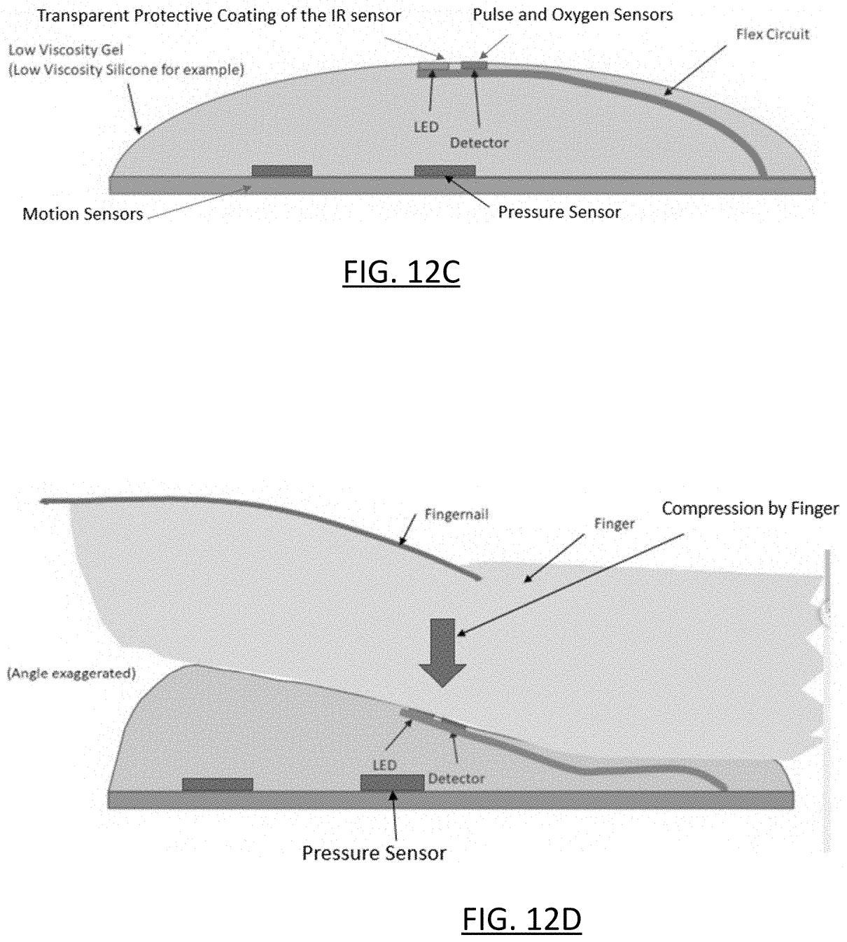

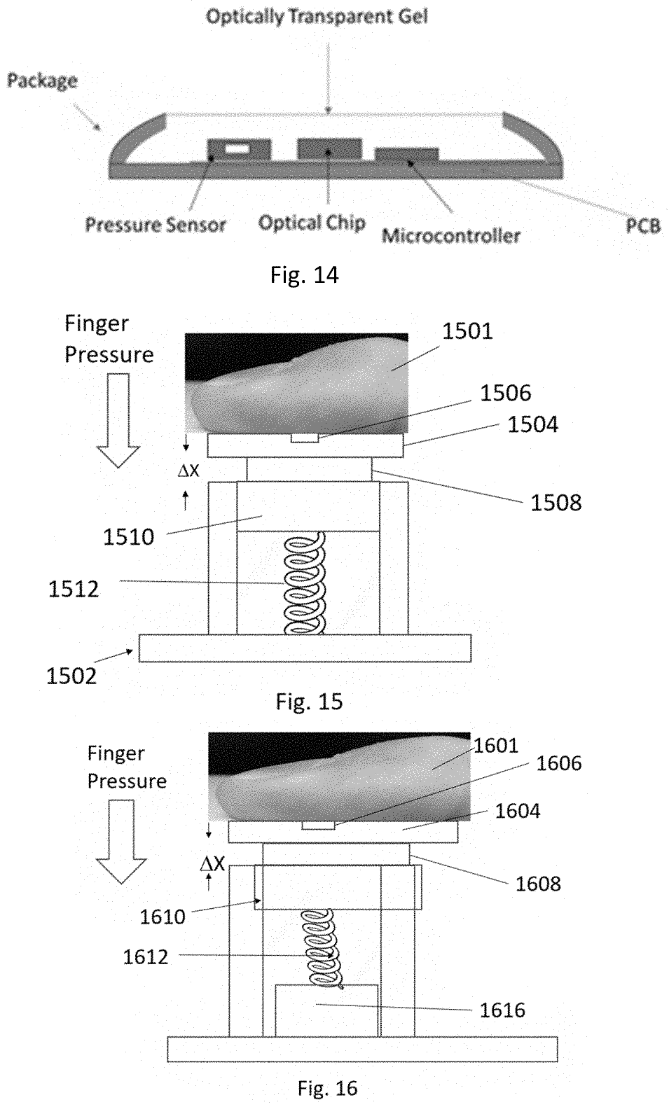

In further implementations, it is possible to use various mechanical designs to improve the ease of use and accuracy of measurements. Ease of alignment a desired artery can be enhanced by ergonomic design of the handheld device, optimized finger grooves, and simplified video and written instructions for use, for example. The main mechanical parameters to vary can include the size, shape, and rigidity of the pressure surface and the overall shape of the device. Applicant has come to appreciate that the angle of the finger can affect the measurements, so a deformable pressure interface (gel-like) can be used, as illustrated in FIG. 12 and in FIG. 14. In another implementation, ergonomically curved or angled device shapes can be used such as those of a computer mouse that more naturally support the finger. The use of adjustable and pop-up or attachable guides can also be used to facilitate finger alignment. Micromachined silicon pressure transducers can similarly be used. For example, a small wedge-shaped and slightly curved bump approximately 5 mm×8 mm wide×5 mm high that is designed to match the generally sloping pad side of the fingertip can be used in some implementations. The bump is preferably high enough to engage the fingertip and allow consistent finger alignment but low enough as to not interfere with the fingernail. The bump can be adjustable so that initially it can be aligned by the user and then locked in place by a mechanism such as a ratchet or pawl.

With reference to FIG. 12A, a platform is provided, such as a PC board including a variety of processing components (not shown) onto which a low viscosity gel, such as low viscosity silicone is provided that encapsulates a flex circuit that terminates in one or more LED elements and a detector. As illustrated in FIG. 12B, a user's finger is depressed onto the structure of FIG. 12A causing the gel mass to deflect and conform to the surface of the user's finger. The LED and detector are then in intimate contact with the finger, facilitating measurement of the user's blood pressure. Alternatively, instead of gel, the flexible body formed by the gel can instead include a fluid filled membrane, such as a silicone or rubber membrane and a silicone oil. If gel is used, the gel body can be formed from a plurality of layers of gel wherein each layer of gel has unique physical properties, such as different viscosities, and the like. If desired, a mixture of opaque gels and optically transparent gels can be used to couple light to and from the skin while minimizing optical cross talk within the sensor structure. Whether gel(s) or a fluid filled membrane are used, in either instance, flat windows or lensed windows can be formed above the emitters and detectors to facilitate clean optical coupling to the tissue.

As illustrated in FIGS. 12C-12D, a further embodiment is provided of a sensor package including a chip or board covered by a gel body or fluid filled bladder that includes one or more LED elements and pulse and oxygen sensors disposed at the end of a flex circuit at a location where a user's anatomy of interest, such as a finger, would contact. A pressure sensor is disposed at the bottom of the assembly under the gel or bladder to sense an application of force by a user and motion sensors can be provided that can see through the gel or bladder to detect movement of the finger. FIG. 12D illustrates deflection of the gel body or bladder under load from a user's finger.

FIG. 14 depicts a variation of the embodiment of FIG. 12, wherein a package, or housing, is provided that includes a bottom surface and defines a peripheral wall that surrounds an opening defined by the peripheral wall at the top of the embodiment. A PC board is disposed in the bottom of the housing including a pressure sensor and optical chip, and a cavity is defined by an upper surface of the lower wall of the housing, the upper surfaces of the PCB and components, and the peripheral wall. This cavity is filled in this implementation by an optically transparent gel and the user places their finger on the exposed upper surface of the gel, which then conforms to the shape of the user's finger to facilitate blood pressure measurement.

In some implementations, improvements are provided with respect to automation of the pressure application, miniaturization of the pressure sensing technology through integration and silicon micromachined sensors, simultaneous sensing of other biomedical parameters for additional diagnostic information and to improve the oscillometric prediction, and improved user interfaces to guide user through the measurement including gamification.

FIGS. 15 to 19 describe further mechanical systems for oscillometric fingertip blood pressure monitoring.

FIG. 15 is a schematic representation of an implementation of a force overload protection device in accordance with the present disclosure. A support 1502 is depicted that supports a pressure plate 1504 having a defined surface area to receive pressure from a user's finger 1501. Plate 1504 includes a PPG sensor 1506 and/or other sensors, as desired, such as a temperature sensor (not shown). Plate 1504 is in turn disposed on top of a force sensor 1508 configured, for example, to detect vertical loading from finger 1501. The force sensor 1508 can be disposed on a displaceable piston or alignment guide 1510 that rides in a vertical guide, wherein the piston or alignment guide rests on a compression spring 1512 having a spring force constant k. In some implementations, the force overload protection system can be designed such that the maximum blood pressure is less than the spring force that can be experienced by compressing the spring, wherein the spring force is provided (at least approximately) by the relation F=kx wherein x is the maximum displacement of the alignment guide or piston in compressing the spring, and such that the maximum spring force is less than the maximum force that the force sensor can detect. As will be appreciated, the spring and mass may act to dampen motion artifacts. The packaging can be configured into a form factor that is squeezed by a user between their finger and thumb, for example. If desired, the force overload protection system can include a visual and/or audible warning when approaching an over pressure, or force, limit on the device itself and/or on a connected smart device or phone. By way of further example, the device can include a removable (e.g., sliding or rotating or fully removable cover and/or a mechanical interlock that protects the force sensor from pressure when not in use. This can be tied to an on/off switch mechanism. A removable cover can also have the advantage of protecting the device from contamination from dust, dirt, or other foreign matter.

FIG. 16 depicts a force overload protection mechanism to automatically adjust pressure and/or assist a user in applying and varying pressure. Like reference numerals indicate analogous system components compared to FIG. 15. As illustrated, this is a variation on the embodiment of FIG. 15. Specifically, this embodiment includes an adjustment 1616 to change the actual length of the spring, placing the spring under more or less compression, resulting in relatively more or less force being required to compress it. For example, a variable wedge can be provided that is slid to adjust the length of the spring to modulate the spring force. Additionally or alternatively, a threaded screw actuated mechanism (coupled to an adjustable mechanical dial, for example) can be used to change spring compression distance and force. A detent mechanism can be incorporated into any of these types of adjustments to provide for a stepwise adjustment of the spring force in order to keep the spring force from changing during measurement. Alternatively, the adjustment can be continuously adjustable. It will be appreciated that the embodiments of FIGS. 15-16 can alternatively be provided with a compressible material in lieu of a compression spring, such as a compressible foam layer formed from high density, medium density or low density polyurethane foam, such as polyester foam or polyether foam.

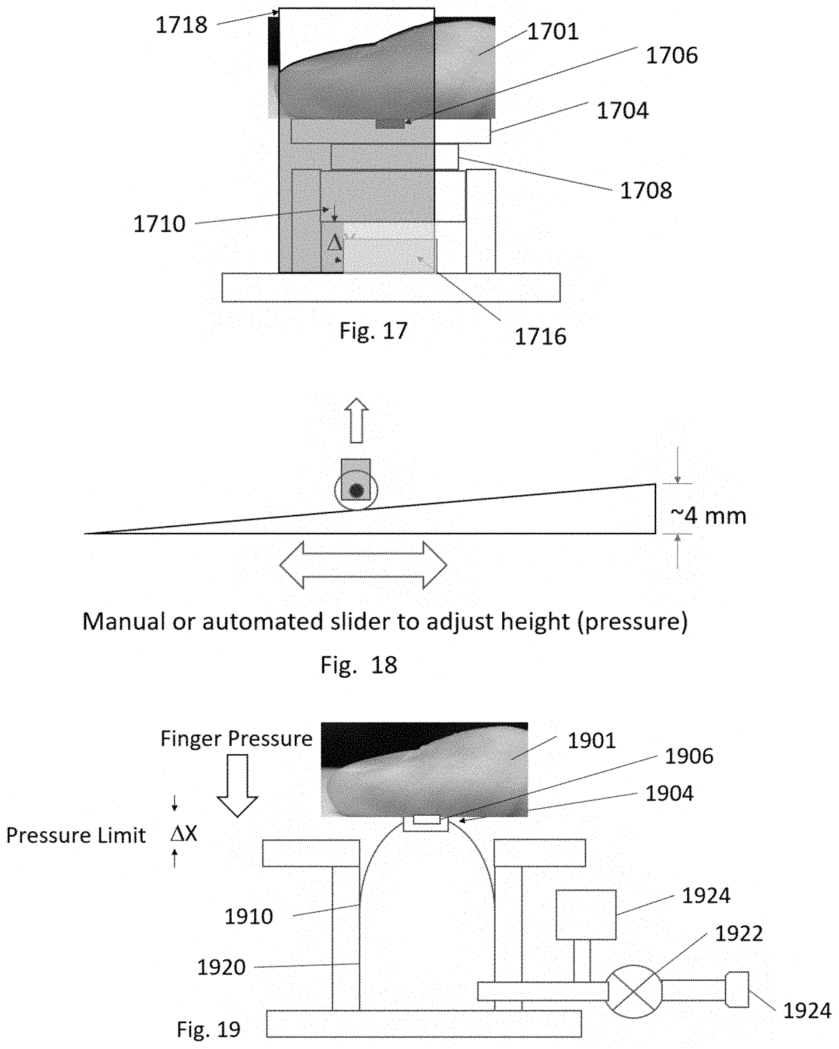

FIG. 17 illustrates Mechanism to assist user in applying and varying pressure. Like reference numerals indicate analogous system components compared to FIG. 15. This embodiment is not illustrated as having a compression spring. Instead, this mechanism is indicated as having a compression adjustment, such as a wedge or dial, as set forth in the embodiment of FIG. 16, and further wherein the fingertip is captured in a finger restraint 1718, such as a groove, that can constrain side to side, back to front, or up and down motion of the finger. The adjustment in height of the pressure plate 1704 can be configured so as to match the range of finger compression, such as about 1 to 4 mm. A distal stop, or other alignment device set forth elsewhere herein, can be provided at the distal end of the finger so as to permit variation of the lengthwise position of the finger to enhance pulsatile signal.

FIG. 18 presents an implementation of a mechanism to adjust height of the pressure plate, and thus applied pressure to the finger (and force sensor). Applicant's experience suggests that a finger only compresses 1-4 mm in the course of a BP measurement by the fingertip oscillometric method. Thus a motion that is as much as 4 mm can be used to supply the required pressure. FIG. 18 shows a sliding wedge configured to meter this compressive force. This embodiment shows a wheel to couple the slide to the sensor platform in FIG. 17. This contact point can also include a low friction slide point or can include a gear on a rack. This shows a linear slide of the wedge, but the wedge can take other nonlinear forms like a circular disk and actuated by rotation (a screw like motion). The adjustment mechanism can include a friction lock or geared coupling to allow more precise control. The motion can be actuated by a user or an automatic actuator can be used having external potential or electromotive energy (such as a spring or a motor).

FIG. 19 illustrates an embodiment utilizing an inflatable bladder 1920 disposed in an alignment guide 1910 to vary the force applied to the finger of a user. As illustrated, this embodiment does not include a large pressure plate, but includes an electronics package disposed on a mount that is attached to a top of a fluid filled bladder. The outside of the bladder is exposed to atmospheric pressure. The user can then compress the bladder 1920 with their finger 1901, wherein the user pushes directly against the bladder and small electronics. A controlled leak is then permitted to flow through a constriction, such as an orifice plate or a valve 1922. A pressure sensor 1924 can be included at a location that experiences the pressure inside the bladder at some location, wherein the illustrated location is intended to only be an example. The pressure can be monitored and logged and be correlated with an applied pressure value for the finger and PPG measurement as illustrated, for example, in FIG. 2C, such as one that is derived empirically for the particular device. If desired, the bladder can be replaced by a fluid filled displaceable piston that includes a relatively weak spring that does not provide much resistance against a user's finger but that instead is configured to re-inflate the bladder after the user's finger is removed. A vent 1926 can be provided to vent excess fluid or other material. Similarly, a small mechanical pump (not shown) can be added to allow electromechanical or user activated mechanical pressure for a more automated measurement experience.

The disclosed force measurement mechanisms can be expected to require calibration, at least during manufacture. This can likely be accomplished empirically, for example, and then correlated through software. This can also be facilitated with precise manufacturing of the sensor itself and/or with a factory calibration of the force sensor component, either at the component level or as part of the assembled BP device during manufacture. If periodic field calibration were required, this can be done in a variety of ways. For example, a user can use the weight of the device pressing against the sensor to accomplish this. Alternatively, a blank, such as a prosthetic having a predetermined shape and dimension configured to fit in the sampling area can be used by inserting it to impart a predetermined load. The device can then be reset by instructing the software to correlate the signals the device generates when the blank is inserted with predetermined values. If the shape of the sensor does not allow the pressure pad to contact a flat surface (like a tabletop), then a removable coupler or stand can be used. The device need not be flat and balanced on the sensor, it can be tilted and a consistent force can be generated for calibration or audit of the pressure sensor.

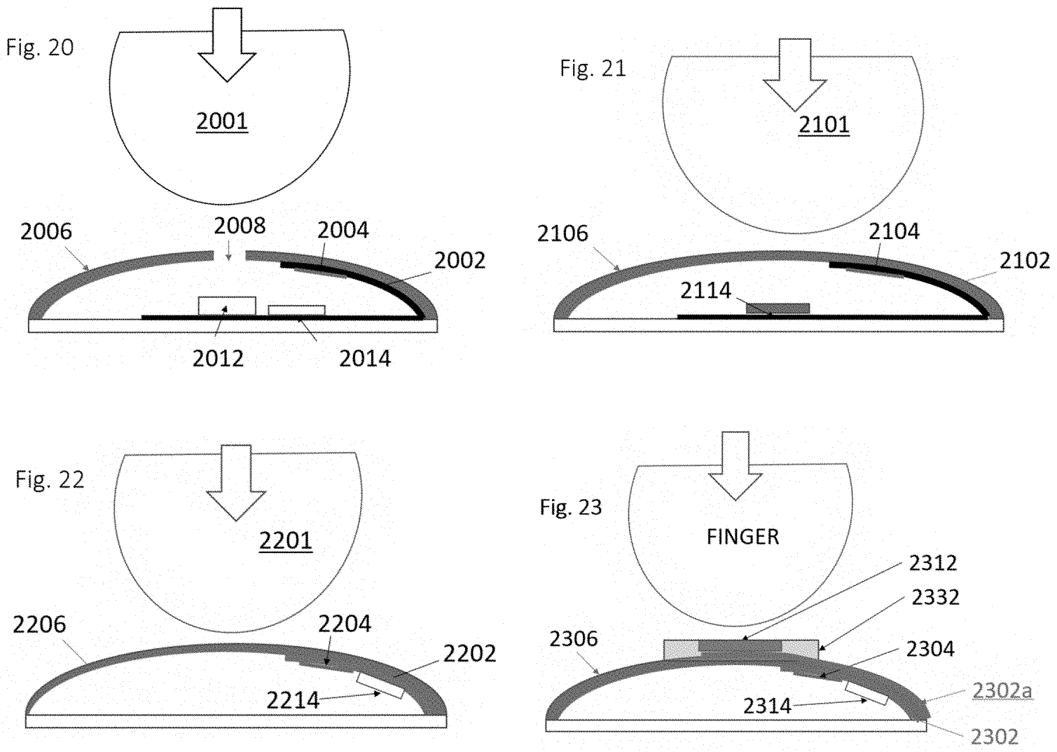

FIGS. 20-25 describe various implementations of strain gauge pressure sensing configurations for blood pressure monitoring.

In one implementation in FIG. 20, a strain gage 2004 is disposed on a deformable surface, such as a silicon strain gauge disposed on a metal dome with an optical sensor as depicted. The strain gage is disposed on a flexible printed circuit board 2002 that is adhered or otherwise attached to a steel dome 2006 or a dome of stiff material. The dome defines an optical window 2008 therethrough to permit transmission of light. Light, such as visible or IR can be transmitted through the window onto an optical chip or optical sensor 2012, such as one including a photodetector. The optical chip is in turn operably coupled to a microprocessor 2014 that is also coupled to the strain gage 2004. The microprocessor 2014 is coupled to a power supply such as a battery and/or a piezoelectric element (not shown) and to a data output line that permits the microprocessor to send a digital signal out to a device to process the signal. As the user presses on the dome 2006 with their finger 2001, the dome 2006 is deflected to an extent, and the deflection is commensurate with a signal generated by or a signal that changes that is passed through the strain gauge.

FIG. 21 illustrates a variation of the embodiment of FIG. 20 that omits a window in the dome 2106 and the optical chip but retains the strain gage 2104 and microprocessor 2114 mounted on the PCB 2102. While not illustrated, a separate optical sensing module and tissue interface, such as that of FIG. 23, can be disposed on top of the sensor. With respect to any of the disclosed embodiments, the pulsatile component of the measured pressure signal can be sued to provide information for the blood pressure prediction algorithm.

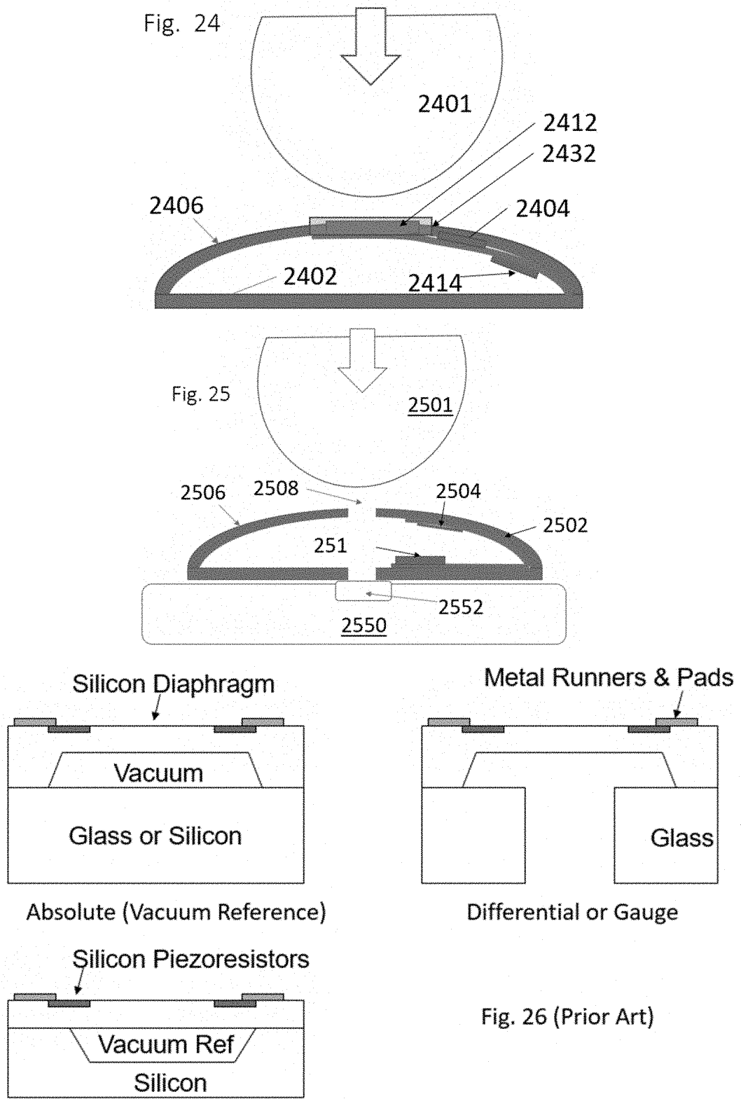

FIG. 22 depicts a further variation wherein the flexible circuit board 2202, strain gauge 2204 and microprocessor 2214 are mounted on the dome 2206. FIG. 23 presents a variation of the embodiment of FIG. 22, wherein a further flexible circuit board 2302a is added to the top of the stiffened dome 2306, and a mount 2332 is attached to the top of the dome 2306 with an optical sensor 2312. The mount holds and protects the sensor 2312, and can be flexible or rigid, as desired. FIG. 24 presents a further alternative embodiment wherein a flexible printed circuit board 2402 is mounted under the dome 2406, and an optical sensor 2412 is mounted above it that protrudes through the dome 2406. The silicon strain gauge 2404 is above the PCB 2402 but below and in intimate contact with the dome 2406. The microprocessor is underneath the PCB. FIG. 25 presents a further alternative similar to that of FIG. 20, but wherein the window 2508 in the dome 2506 has a line of sight to a camera 2552 of a mobile device 2550, such as a smartphone. In accordance with a further embodiment, a pressure sensor incorporated into a smart phone can be used to sense the pressure.

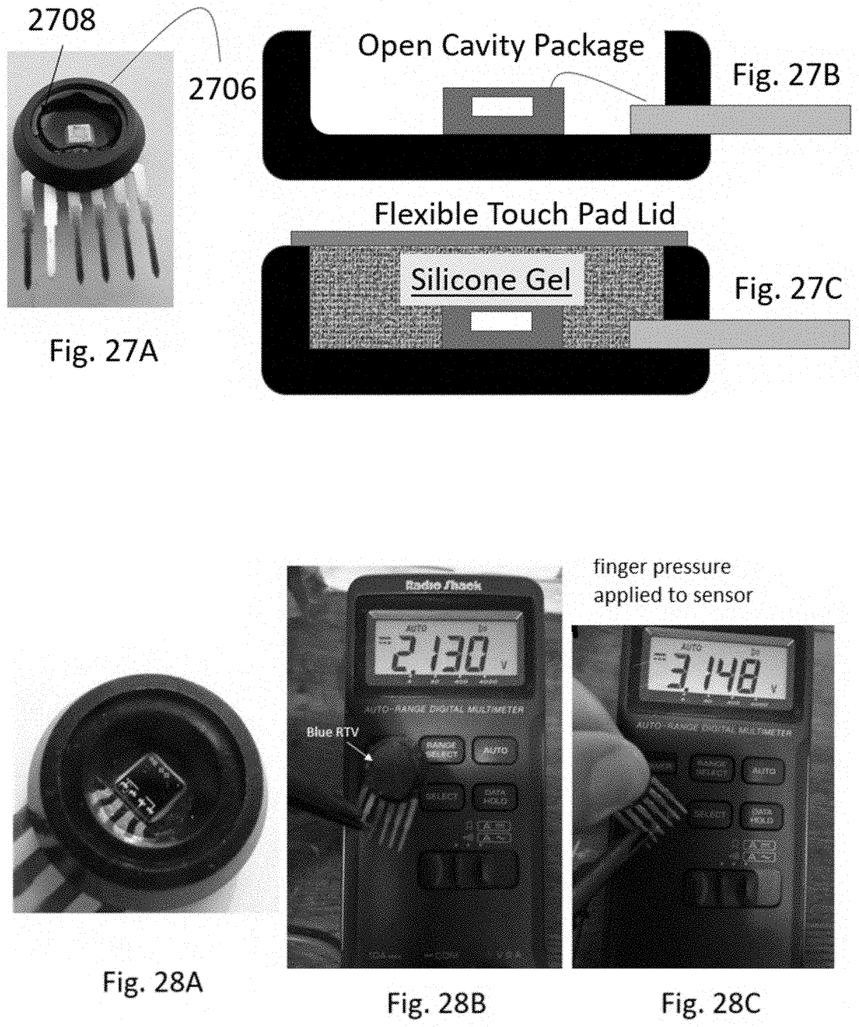

FIGS. 26-37 set forth illustrative embodiments of silicon micro-machined pressure sensing configurations for blood pressure monitoring. FIG. 26 sets forth various pressure sensors known in the art. FIG. 27 illustrates an implementation of an absolute pressure measurement device that includes a leadframe package that defines an open cavity therein. The cavity includes a bottom wall with a sensor package 2706 (e.g., absolute pressure sensor) that is electrically coupled to a plurality of conductors exiting the package and defines an opening 2708 therein that can receive an optically conductive gel material, for example. The device can be an open cavity as indicated in FIG. 27B that receives a fingertip therein to contact the sensor package. Alternatively, if desired, the cavity can be filled with a flexible medium such as a silicone gel as illustrated in FIG. 27C and fitted, if desired, with a flexible touch pad lid that can receive a fingertip. Signals can then be transmitted from the fingertip to the sensor and from the sensor to the fingertip, if so configured, through the gel or other medium and the lid, if so provided. It should be noted that all implementations herein that use a gel medium can be modified to include a fluid in a bladder, and that all embodiments herein including a fluid in a bladder can be replaced by a gel or other medium, such as an elastomeric medium that can transmit optical signals. FIG. 28 illustrates an embodiment of a prototype of such a gel filled embodiment that is filled with a blue RTV as indicated in FIG. 28A and provides a signal under finger pressure as indicated in FIGS. 28B-C.

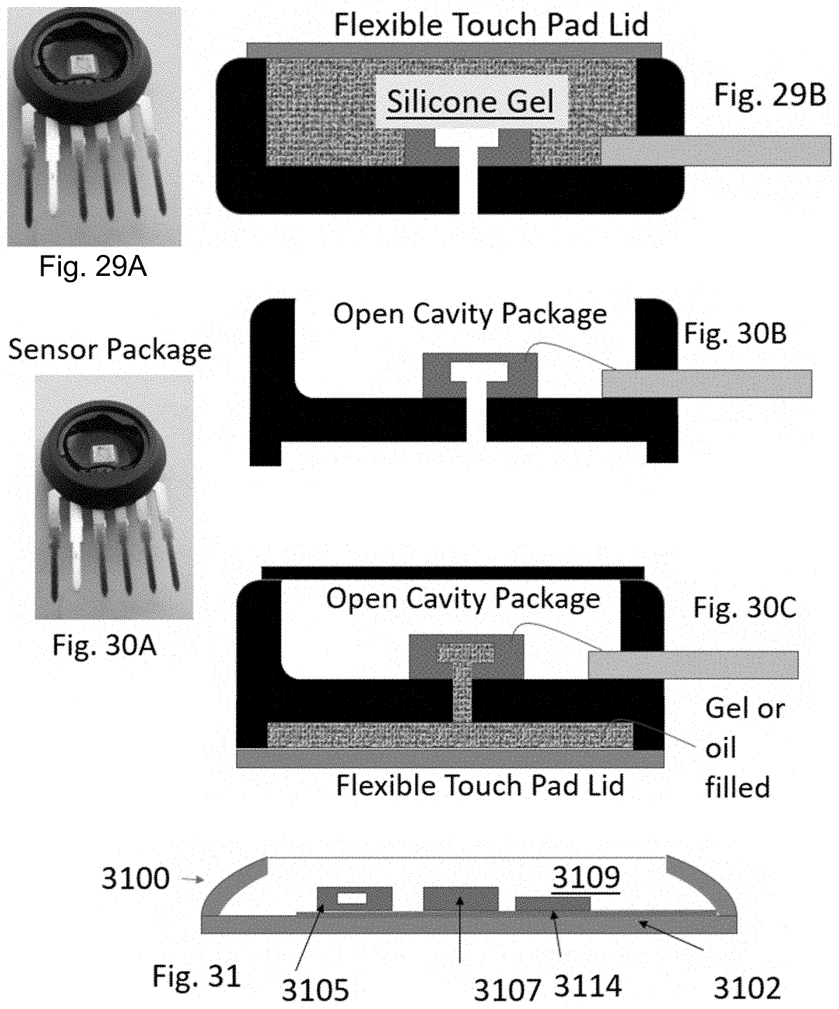

FIG. 29 illustrates a variation of the embodiment of FIG. 27 that includes a differential pressure measurement arrangement that defines a passage through a bottom side of the leadframe package to a backside of the pressure sensor, to permit a differential pressure measurement to be taken of pressure applied to the touch pad lid. FIG. 30 illustrates implementations of a backside sensor leadframe. FIG. 30B illustrates a compartment containing a pressure sensor that is open to the atmosphere on the front and back. The sensor is mounted in a relatively larger cavity and the lower cavity is connected to an underside of the package by way of a channel. The embodiment of FIG. 30C adds a gel or silicone oil to the cavity formed by the lower cavity that passes through to the underside of the pressure sensor and adds a touch pad lid on top of the gel to hold the gel in place. A cover can similarly be placed over the open cavity containing the sensor chip.

FIG. 31 describes an implementation 3100 of an absolute pressure sensor device similar in configuration to the sensor package of FIG. 14. The package includes a compartment defined by a flexible pad or base wall and a peripheral wall cooperating to define a compartment with an open top. A printed circuit board (PCB) 3102 is disposed on an upper surface of the bottom wall. The PCB in turn includes a pressure sensor 3105 and an optical chip 3107 operably coupled to a microprocessor 3114. The cavity is filled with an optically transparent gel 3109 that, if desired, can also be provided with a cover plate that includes an optically clear portion.

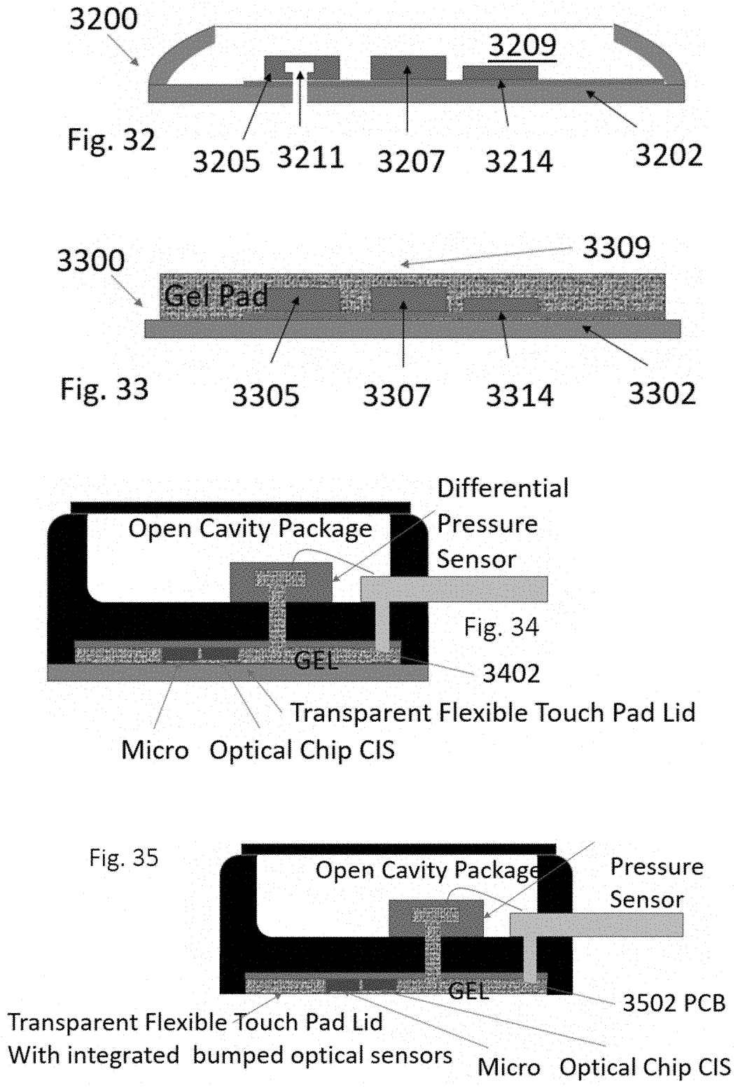

FIG. 32 presents an alternative design 3200 of the embodiment of FIG. 31 that is configured to measure a differential pressure between the surface of the gel 3209 (or a cover surface over the gel) and atmospheric pressure. Specifically, a vent channel 3111 is formed between a bottom surface of the package 3200 and an underside of the pressure sensor 3105, such that the pressure sensor 3105 experiences atmospheric pressure on its underside and pressure through the gel above, such that a differential pressure measurement results from the pressure sensor. The pressure sensor can also be part of the flexible lower pad along with the optical sensor.

FIG. 33 presents a further embodiment of a sensor package 3300 in accordance with the disclosure that includes a flexible base layer (e.g., of a polymeric or composite material) that includes a PCB 3302 disposed thereon, and further wherein the PCB 3302 includes a pressure sensor 3305 and an optical sensor 3307 operably coupled to a microprocessor 3314. A gel pad 3309 is molded over, or otherwise disposed over the components. The system can sense absolute pressure as pictured, or a vent channel as with embodiment 3200 can be formed to an underside of the pressure sensor to permit sensing of a differential pressure. The pressure sensor can also be part of the flexible lower pad along with the optical sensor. Leads in the form of solder bumps, for example, can be used to interface with a further receptacle or other PCB.

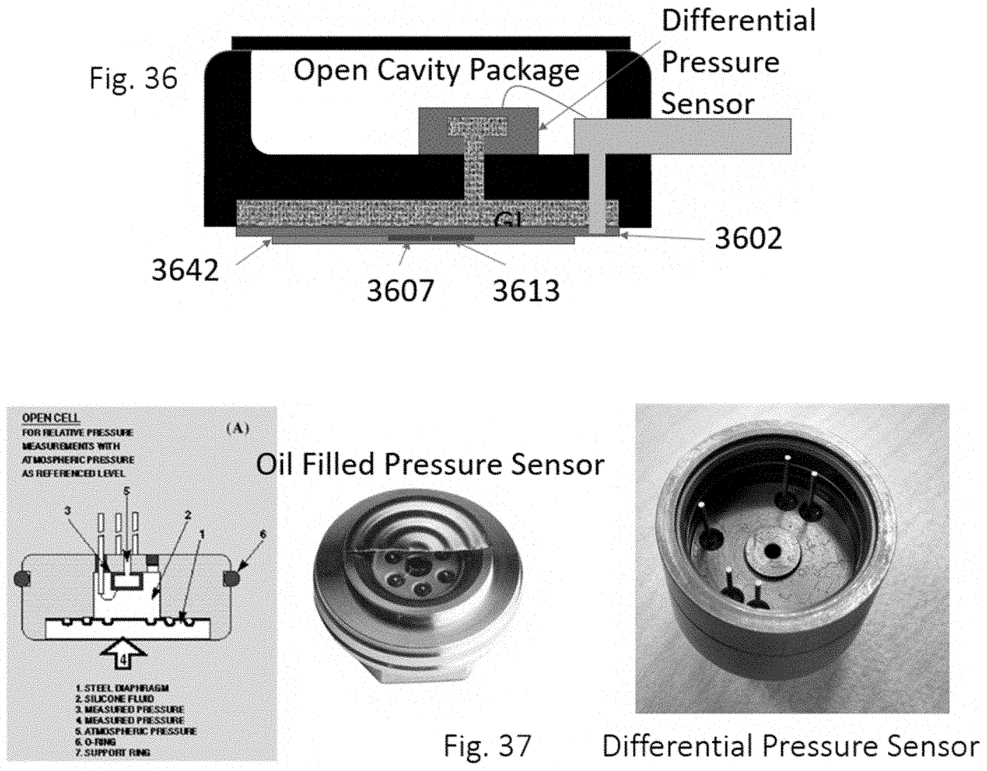

FIG. 34 discloses an open cavity package having a housing that defines first and second chambers surrounded by vertical walls and having a respective bottom wall in the upper chamber, and a top wall in the lower chamber in respective top and bottom sections of the housing. The bottom wall of the upper chamber can define a passage therethrough to connect the chambers. The upper chamber includes a sensor package in the form of a differential pressure sensor. The sensor includes a gel interior that passes through the passage formed through the bottom wall into the lower chamber. The lower chamber includes a PCB 3402 mounted on its upper wall, or ceiling, that includes a microprocessor coupled to an optical chip and the pressure sensor. The lower chamber further includes an optically clear gel or other optically clear flexible medium that surrounds the optical chip and is coupled to the gel portion that extends toward an underside of the differential pressure sensor. The lower chamber is covered by a transparent flexible touch pad lid and the upper chamber can also be covered by a cover portion. The upper chamber is preferably vented to atmospheric pressure to permit a differential pressure measurement to be taken. FIG. 35 provides a variation of this embodiment that uses a thin film cover over the lower chamber. FIG. 36 illustrates a further variation wherein the lower chamber is filled with gel along with the passage to the underside of the pressure sensor, and further wherein the PCB 3602 is mounted over the gel layer on the lower face of the device, and the optical sensor 3607 and sensor interface electronics 3613 can be coupled to the pressure sensor and the leads. A passivation or protective layer 3642, which can be flexible, rigid or a combination of both can be disposed over the PCB 3602 and the optical sensor and interface electronics so as to improve or optimize the interface for the finger of a user. The PCB 3602 can be connected to the pressure sensor, but can also be connected externally to a further device.

FIG. 37 illustrates an example of a welded, e.g., metal, corrugated diaphragm used for pressure measurement that is typically filled with silicone oil. Such a device can be modified to use the electronics as described above, and the material can be varied and be injection molded plastic instead of metal. Similarly, silicone oil can be used as a medium in the above described embodiments rather than gel or silicone, for example.

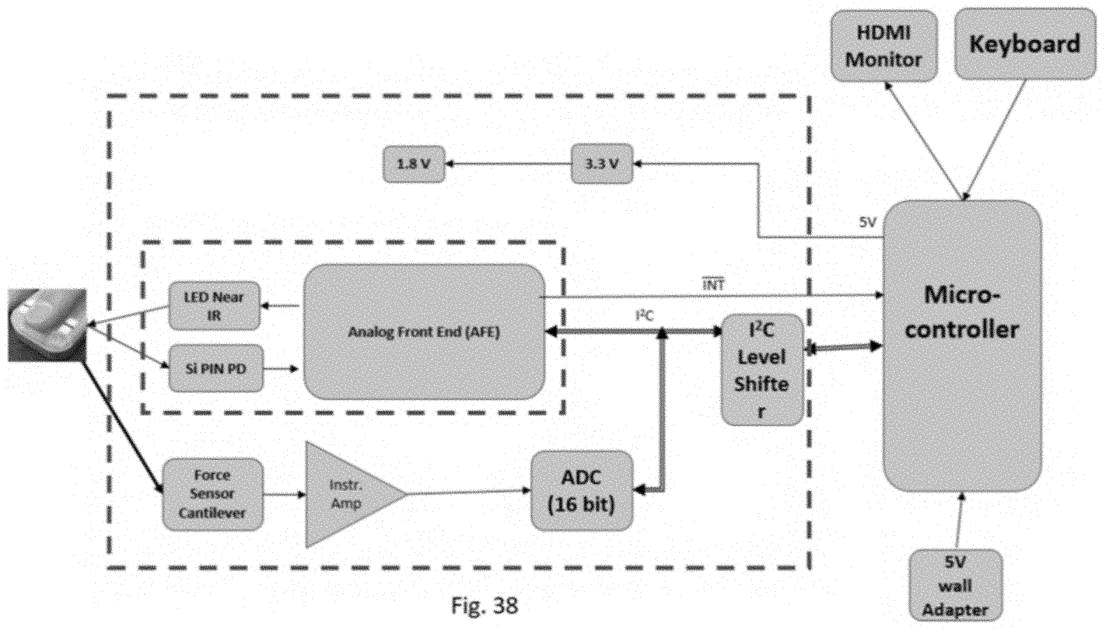

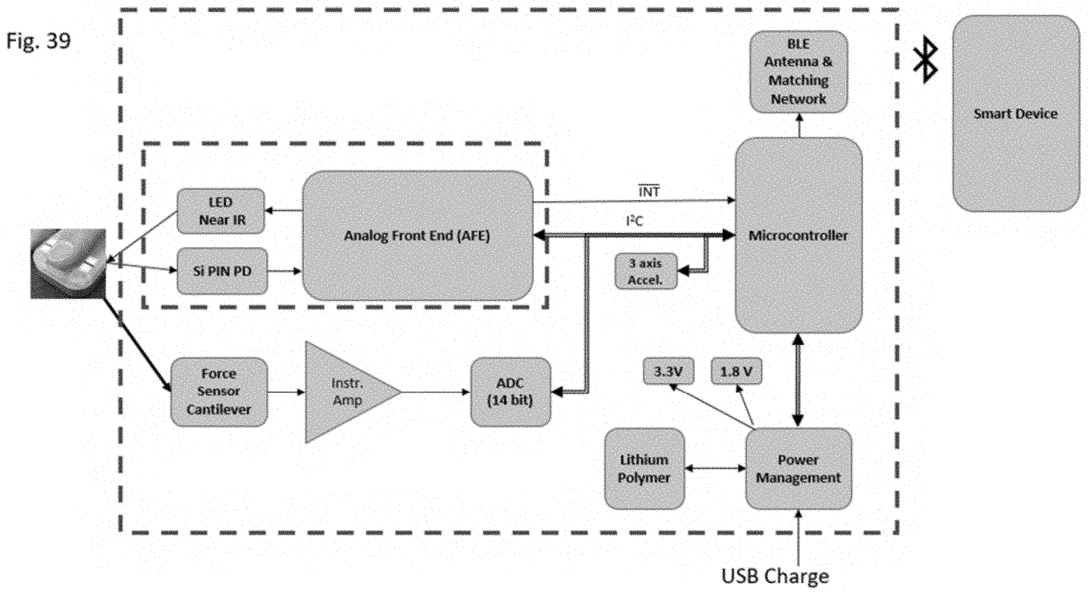

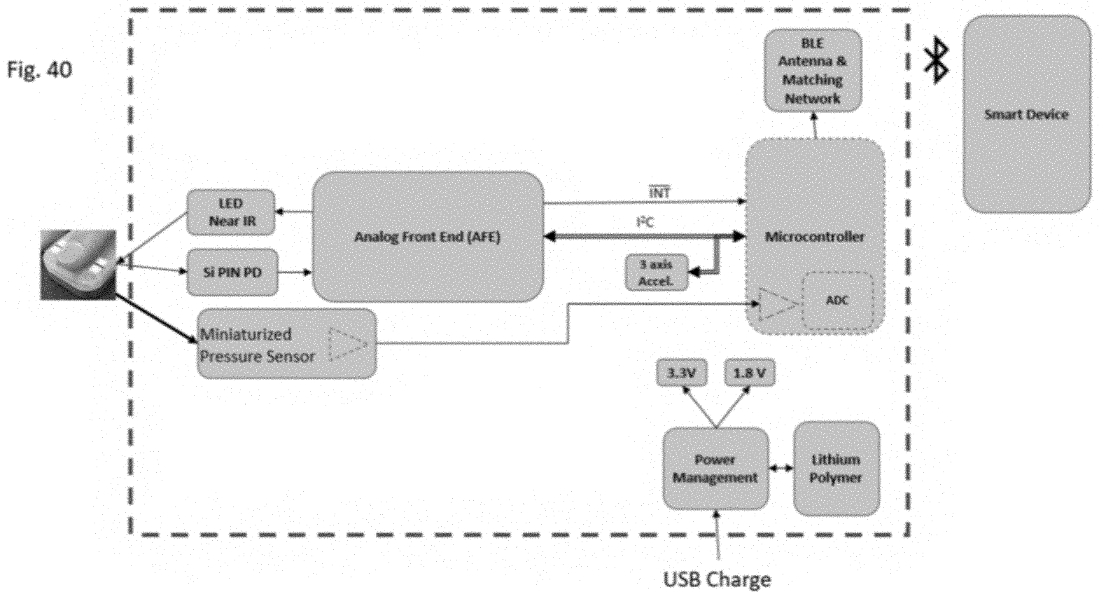

In further accordance with the disclosure, three illustrative electronic architectures for the oscillometric fingertip blood pressure monitoring system are shown in FIGS. 38-40. The oscillometric sensing is done by measuring the applied pressure with a force sensor and the pulse amplitude in the finger using a photoplethysmogram (PPG) sensor. This basic sensing architecture can be used to monitor more physiologic parameters than just blood pressure and heart rate, and additional sensors can be added to facilitate simultaneous and/or sequential measurement of still more physiologic and environmental parameters that have additional clinical significance as well as being helpful in providing information to better predict blood pressure (improved accuracy and reliability). The improved accuracy can be facilitated by adding independent information to the blood pressure prediction computation and the reliability can be increased by detecting potential error conditions in the measurement. These alternate measurements are described below.

Pulse Oximetry

PPG sensors can be used in the disclosed embodiments to measure blood pulse volume. It is further within the scope of the disclosed embodiments to add measurement using one or more additional wavelength(s) of light to obtain a blood oxygenation signal. For example, some implementations include both a red and IR LED. Some implementations use the IR LED to measure blood volume. However, additional implementations can utilize the red LED to make an independent measurement and perform computations to predict blood oxygenation. Similar integrated chips can be used with or without external LEDs and photodetectors accordingly. Blood oxygenation is a useful diagnostic parameter. However, additional information provided by utilizing additional wavelengths can provide additional information that can increase the accuracy and reliability of blood pressure predictions. An example of this, in some implementations, is that the pulsatile change in blood oxygenation or oxygenated versus deoxygenated hemoglobin measured by the multi-wavelength PPG measurements can be utilized to better identify the pulsatile arterial signal which can be correlated to the oscillometric effect used to predict blood pressure.

Independent Mechanical Measurement of Pulse

The pressure or force sensor used in certain implementations is used to assess pressure applied to the fingertip. There is however some pulsatile component in the force measurement that is caused by the volume expansion due to each blood pulse which in turn causes a transient increase in measured pressure even with constant force applied by the user. This pulsatile signal is very visible in many subjects and has an oscillatory amplitude profile that tracks the PPG pulsatile signal. This mechanical signal can be used as an independent input to the computation to predict blood pressure which Applicant believes can improve blood pressure prediction.

Accelerometer

Some embodiments of the Touch BP device can include an accelerometer. The accelerometer can be used to measure the orientation of the device in order to compensate for tilt in the force sensor. This compensation is helpful because the “zero” of the strain gauge that measures the applied force on the fingertip depends on the self weight of the pressure sensor head. By computing the exact orientation of the axis of the force gauge relative to the acceleration due to gravity, it is possible to compensate for changes in the zero of the force gauge. Because micromotion artifacts can disrupt a PPG signal, this accelerometer can also be used to detect motion artifacts. If an abrupt motion is detected, then the device can correct the PPG reading or instruct the user to apply pressure in the range of the artifact for additional time to “replace” PPG vs applied pressure data that was noisy or corrupted.

ECG (or EKG)

Electrocardiogram (ECG) signals can also be measured by using a sensor chip that includes a dedicated analog front end (AFE) having a multichannel PPG sensor circuit as well as a dedicated two wire ECG sensor circuit. The ECG signal can be measured independent of the blood pressure measurement, or simultaneously. Two electrodes are supplied to make contact to fingers on opposite hands of the subject. This can be accomplished, for example, by putting a first of the two electrodes in a first location, such as around the PPG sensors on the pressure plate, and the second of the two electrodes in a different location, such as the side, end, or bottom of the device where a finger from the opposite hand can be held on the device during a normal oscillometric fingertip blood pressure measurement.

ECG alone is a very valuable diagnostic tool for detecting episodes of arrhythmia and other unusual heart beats. However, coupling this measurement with the normal oscillometric measurement data can improve the accuracy and reliability of BP prediction. For example, having simultaneous ECG signals and PPG signals permits pulse rate, heart rate, and pulse transit time values to be computed and this can provide an independent input vector to the blood pressure predicting algorithm. Likewise, the simultaneous PPG and pressure data coupled with the ECG signal can have higher diagnostic predictive value than either measurement independently when it comes to cardiovascular disease and events such as strokes and heart attacks (MI). Because this system is well suited for frequent at home and on the go monitoring, there is significant opportunity to collect data over large populations and build predictive algorithms. The ECG signal can be used as a basis lock-in synchronous detection of the PPG pulse to detect pulsatile signal with greater a signal to noise ratio.

Temperature

Skin temperature is another diagnostic measurement that can be added to the system. A thermopile or surface thermistor can be used to measure temperature simultaneously as the finger lays against the device at the base or even at the fingertip where the blood pressure is measured. It can also be placed elsewhere on the device to measure the temperature separate from the blood pressure measurement. Not only does body temperature provide important diagnostic information like detecting fever, it also provides information about perfusion which is important to a fingertip blood pressure measurement. Because perfusion can affect the pulsatile signal and thus have an effect on the oscillometric signal, the temperature can be used to improve the oscillometric BP predictions across a wider population. The temperature sensor can also be located in such a way that it can help verify proper alignment of the finger on the device for a measurement. This can also be helpful in detecting Reynaud's syndrome.

DC Component of the PPG Signal

Typically pulse measurements and oscillometric measurements concentrate on AC or the time varying component of the raw optical signal from the PPG sensor. However, as some disclosed implementations can also measure the DC component, the algorithms for predicting the BP can also use the DC component of the signal. The absolute DC signal or the ratio of the AC/DC signal for example can be used to detect signal quality, or this ratio can provide independent information that can improve prediction accuracies or robustness.

The disclosure further provides methods and apparatus for improved finger/device interface in fingertip oscillometric blood pressure (and other) physiologic monitoring. These can include, for example, a soft/conformal pressure pad to provide even pressure with varied finger shape and hardness, an alignment guide for finger placement, device guided alignment optimization for the finger or other anatomy of interest, multiple emitter/detector pairs using all or optimum pair elongated pressure pads, and a pressure pad with an elongated pressure ridge optimized to cross the transverse palmer arch artery.

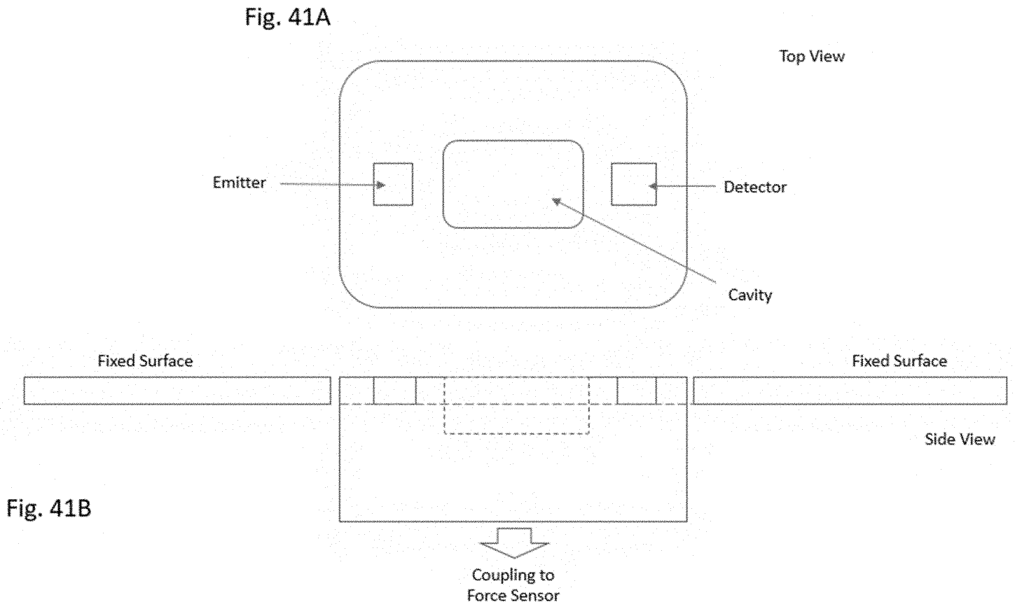

FIG. 41 describes an illustrative implementation that utilizes a void defined between the PPG emitter and detector to increase pulsatile signal. Specifically, a void can be defined on the device that creates a small area where capillaries do not collapse yet the overall compression of the artery occurs. An open connection is maintained between the compressed artery and the uncompressed capillaries between the emitter and detector, which increases the pulsatile component of the PPG signal. This can be especially helpful in people with decreased perfusion. This technique can be applied to other more traditional PPG measurements like pulse and blood oxygenation.

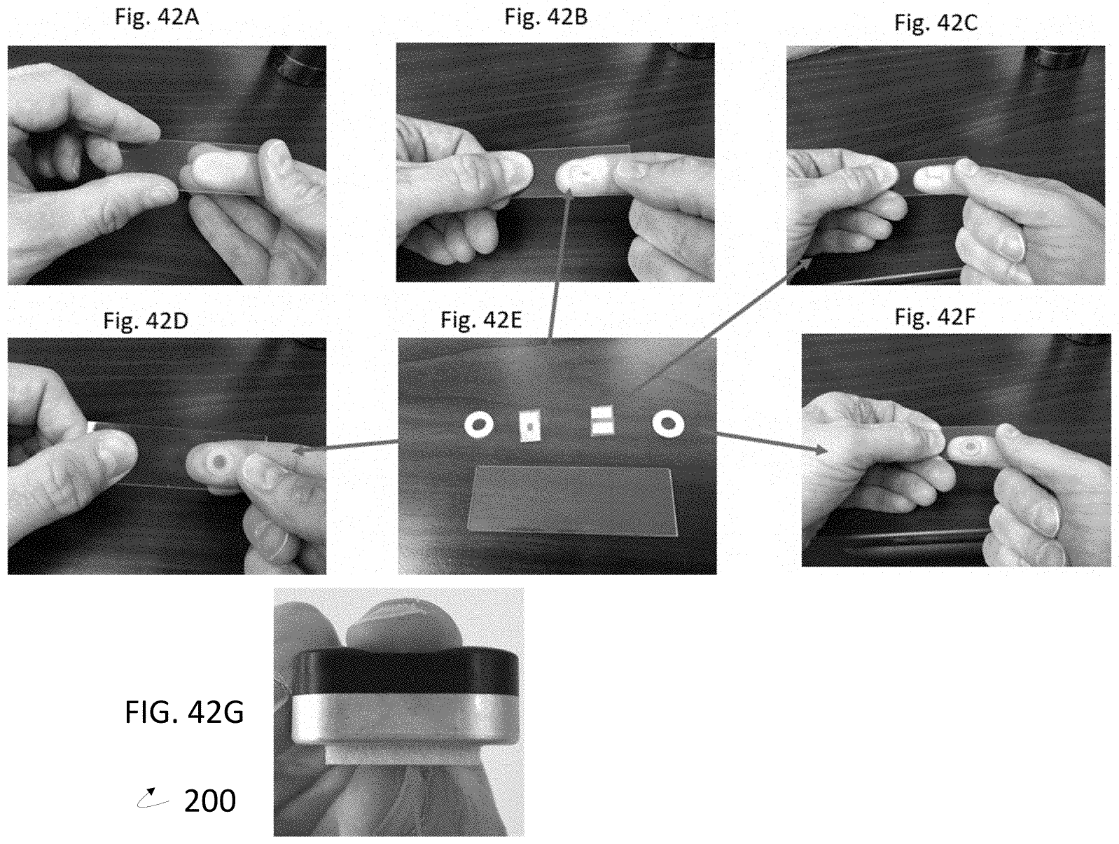

As an illustration of this, FIG. 42 illustrates the pooling of blood in capillaries in a void in the compression plate. FIG. 42A illustrates the presence of no aperture, showing pale white skin tone demonstrating the collapse of capillaries. FIG. 42B illustrates a small rectangular aperture, showing darkened skin in the aperture, wherein the aperture is about 3.8×2.5×1.4 mm. FIG. 42C illustrates a small slit aperture of dimensions that are about 8.4×2.4×1.4 mm, also showing darkened skin in the aperture demonstrating pooling of blood. FIG. 42D illustrates a 6.4 mm diameter 1.6 mm deep aperture, and FIG. 42F shows a circular aperture that is about 5.6 mm in diameter and 1.0 mm deep.

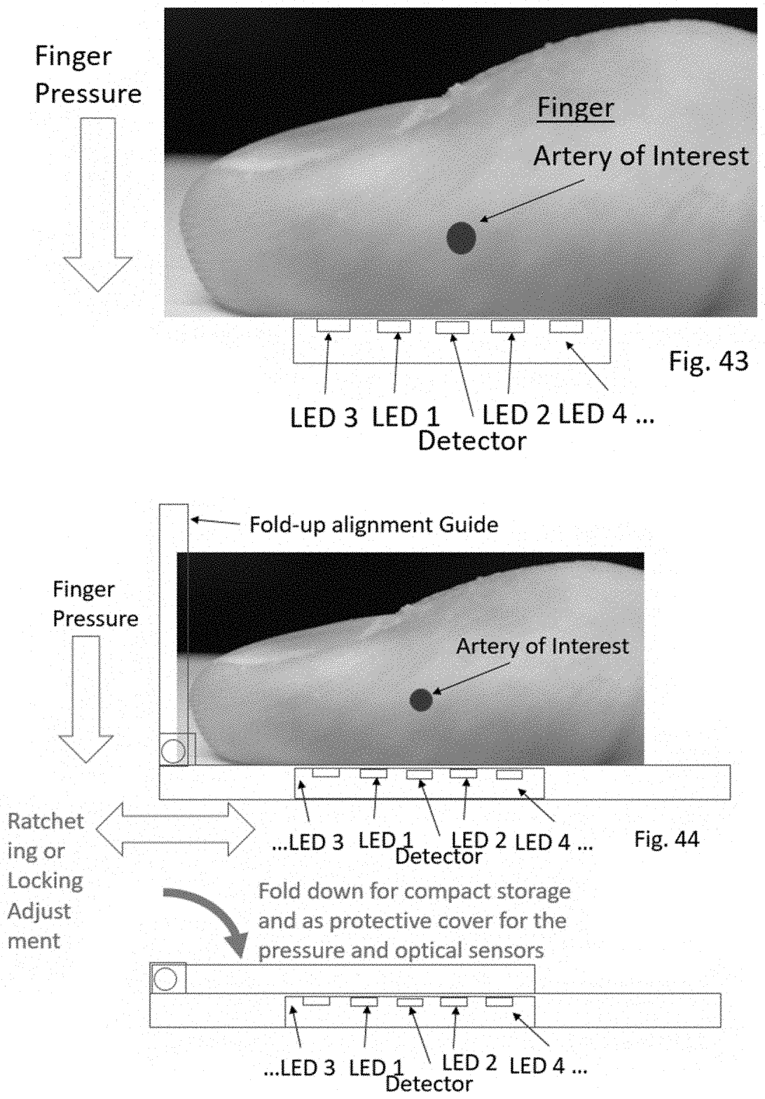

FIG. 43 describes an arrangement of multiple emitters/detectors to optimize alignment of an anatomy of interest. While a fingertip is illustrated, it is possible to use other anatomical locations as mentioned elsewhere herein. The pulsatile component of the light that propagates through the tissue is the primary signal of interest for oscillometry because the pulsatile signal is proportional to pulse blood volume. Experimental measurements indicate that the pulsatile component is sensitive to proper positioning at the scale of approximately 1-3 mm. Obtaining the optimum signal will very slightly from person to person due to physiological/anatomical differences including variations in tissue optical absorption and scattering and variation in depth and position of the artery of interest (the transverse palmar arch artery for example). Even if the optimum position for a detector and emitter pair are identified for a given subject, there can still be considerable variation between measurements due to variation in placing the finger on the sensor. In the embodiment of FIG. 43, multiple optical emitters and a single detector are used to help minimize misalignment with the artery. For example, when the finger is initially placed on the sensor the signals from all combinations of sensors can be compared and the emitter that gives the highest pulsatile component can be used to make the measurement. In some implementations, the initial comparison can be done at a time when the pressure being maintained is relatively constant. Although four emitters and one detector are illustrated in FIG. 43, it will be appreciated that there can be more (e.g., five, six, seven, or more) or less (one, two or three) emitters and there can also be multiple detectors (e.g., two, three, four, five, or more). Multiple signals can be collected from multiple emitter/detector pairs to generate independent data to predict or estimate blood pressure or detect unwanted changes in finger position during a measurement.

FIG. 44 illustrates embodiments of mechanical alignment guides to help aid finger placement, or for the placement of other anatomy, as appropriate. Adjustable and fixed alignment guides can be used to ensure consistent alignment of a subject's finger or other anatomy. In FIG. 44, a flip-up guide that pivots around a pin, or hinge, that acts as a finger stop is illustrated. The flip up guide is advantageous as it reduces the profile of the device for storage when it is not in use and it can act as a protective cover for the pressure and optical sensors while the device is not in use. The alignment guide can be adjustable along the axis of the finger so that the artery of interest is optimally aligned with the pressure and optical sensors regardless of anatomical differences between subjects (for example length of finger, fingernail length, and artery position relative to the fingertip). Once the guide is in a suitable position, it can be locked in place. For example, a ratchet lock can be used that is released to move after pushing or squeezing a release trigger, for example, and then once in place the release trigger is actuated to lock the alignment guide in place. This can be accomplished, for example, with a slide and friction lock or a geared or ratcheted mechanism with a release or lock. In use the subject can be guided to change the finger position while maintaining a target pressure. The pulsatile signal can be monitored during this time to find a position of maximum or increased pulsatile component and this can be the point where the alignment guide is locked. The device can be programmed to signal the user to lock the alignment guide at an optimal or suitable position. Although the illustrated alignment system is depicted with multi emitters and detectors, it can be used in a system with any number of emitters and detectors (as with the embodiment of FIG. 43) including only one emitter/detector pair. The alignment need not be a pure vertical stop. The alignment can be a fingertip shaped depression or a small ridge that does not reach the fingernail. Not touching the fingernail can eliminate the possibility of alignment changes due to changes in fingernail length. For example, a small wedge shaped and slightly curved bump approximately 5 mm×8 mm wide×5 mm high that is designed to match the generally sloping pad side of the fingertip can be sued. The bump can be high enough to engage the fingertip but low enough as to not interfere with the fingernail Alignment can also be made to reference to other points on the finger. For example, a small ridge can align with the knuckle groove of the finger joint.

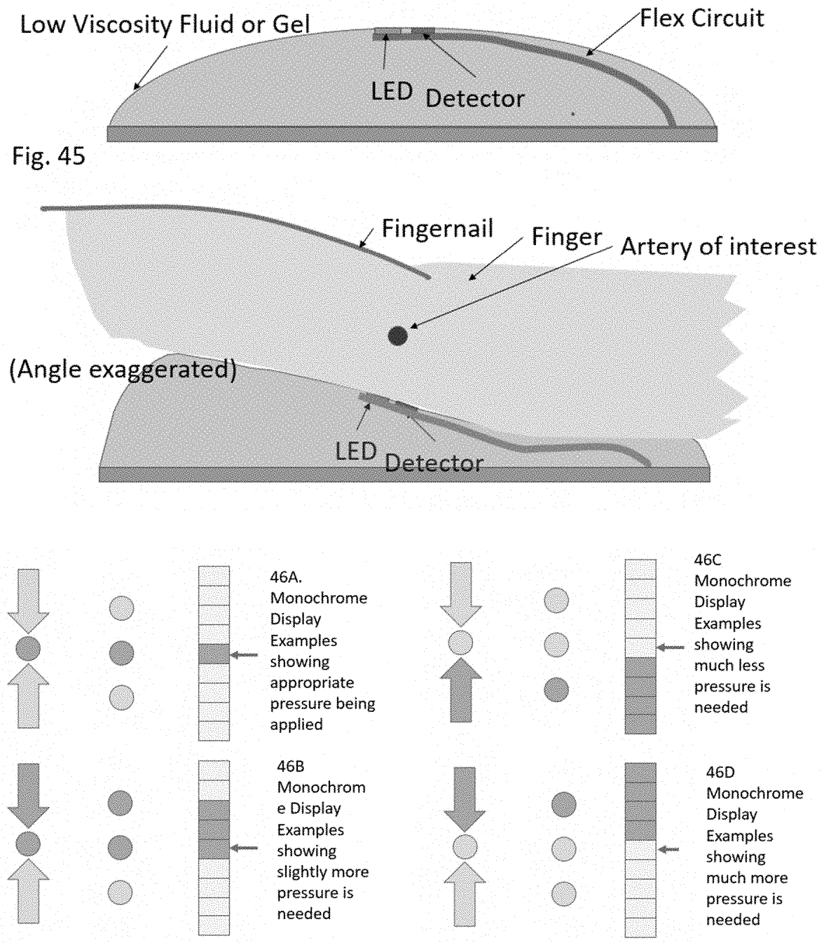

FIG. 45 illustrates an embodiment of the Touch BP device having a soft and/or conformable pad for more uniform pressure application on the finger or other anatomy of interest. As illustrated in FIG. 45, a deformable pressure pad can be used to address the effect of orientation and physiological differences in finger anatomy on the accuracy of measured blood pressure. A pad of low viscosity gel such as a silicone or an oil filled bladder (e.g., filled with silicone oil) can permit pressure to be applied more evenly on the underlying arteries. In the example of a fingertip, the distal phalanx has a natural slope getting thinner toward the tip which creates an uneven pressure on the tissue that is compressed between it and a flat hard surface. The distal phalanx is also rounded and significantly smaller that the external dimensions of the finger pad which can also create pressure gradients when the finger is pressed against a flat surface. By conforming more to the finger external and internal bone shape a soft, deformable surface can even out applied pressure and help reduce the variation in the internal pressure at the artery as compared to the applied pressure measured by the instrument. This can reduce the measurement variation caused by small changes in alignment and variations in anatomy. In FIG. 45, this is shown with one detector emitter pair, but it can be used with multiple emitter/detectors as discussed earlier.

The measurement of pressure can be accomplished by measuring the pressure (or force) of the base on a force sensor (like a load cell as discussed elsewhere in this application). This can also be applied to the silicon micromachined pressure sensing technologies also discussed hereinabove. In this latter case, the pressure sensor can be embedded in the gel or oil to measure the pressure. The soft pad can be constructed with a predetermined shape that molds to the average curvature of the finger. Various embodiments herein can further include an indentation(s) and/or pads, such as a deformable pad on the housing for a thumb grip, as illustrated in FIG. 42A, wherein device 200 is pictured in end view with an index finger placed in the upper finger groove for measurement, and the user's thumb is placed on a conformable pad (e.g., of foam) on the opposing side, or bottom, of device 200. The soft pad can have a shape that implements a void that enhances PPG signal as discussed above with respect to FIGS. 41-42 for example. It can be advantageous to make the pressure pad elongated along the finger axis to better match the shape of the finger.

The disclosure further includes methods and user interfaces to guide applied finger pressure including gamification and adaptive pressure guidance. In some implementations, adaptive pressure slopes can be utilized. For example, it is possible to set the pressure slope (e.g., to increase or decrease) based on heart rate. A faster heart rate means a higher possible slope. In order to obtain a high signal to noise ratio one needs a certain number of heart beats per change in applied pressure to accurately describe the oscillatory envelope. However, it is not desirable to set the slope of the pressure gradient arbitrarily low because this increases the measurement time which reduces subject convenience. One example of an approach is to base the slope of the pressure gradient on the subject's measured heart rate. A faster heart rate can allow higher pressure slope and therefore reduced time of measurement for a given blood pressure profile. By making it adaptive in this manner, the device can reduce measurement time for an individual. The average adult resting heart rate is typically in the range of 60-100 BPM, but there are still people with heart rates lower or higher than average. In this way a wide range of pulse rates can be accommodated with a reduced measurement time while maintaining measurement accuracy. In another implementation, it is possible to reduce measurement time by starting the measurement at a point that is based on diastolic readings from previous measurements. In one example, for two subjects, one can have an average diastolic blood pressure (DBP) of 60+/−7 mm-Hg and another can have an average diastolic BP of 100+/−15 mm-Hg. Once the device has enough data to confidently estimate DBP, then as an example the start pressure for the first subject can be set to 40 mm-Hg and 70 mm-Hg. Thus, the sweep time can be significantly reduced, especially for the subject with higher DBP. In the unlikely event that the current DBP is unusually low then the user can be guided to repeat the measurement with a lower starting point or directed to obtain data at the lower pressure to supplement the original scan.

The quality of the data can also be increased by actively changing the requested slope based on a subject's performance, quality of data, and oscillometric envelope in real time. For example, if the quality of data is poor in an important area of the oscillometric curve because of, for example, weak pulsatile component or motion artifacts, then the pulse slope can be adjusted to revisit this area or dwell longer in that area. Similarly, if the subject has difficulties maintaining the pressure slope and effectively skips through a critical area of the oscillometric envelope then the target slope can be “reset” at the current point and the user guided via computer implemented instruction to apply a new pressure slope from this starting point. This can help avoid erratic finger pressure changes as the user tries to quickly correct. This can be especially helpful for users who have trouble maintaining a constant pressure profile. In an example where a person is slow to react to the need to increase pressure and ends up near the low end of the acceptable range, rather than encourage the patient to rapidly correct for this by moving up rapidly to a programmed slope it can produce smoother data to reset the desired path to start near the current position. In another implementation, the system can track the ability of a person to track the pressure profile. As a measure of hand eye coordination, this can be used as a long term indicator of cognitive health and as a diagnostic tool for cognitive degeneration.

In some implementations, and as illustrated in FIG. 46 methods, devices and graphical user interfaces and supporting machine readable programs are provided to guide pressure measurement on the device. For example, it may be advantageous for the user to make the device work independently from a smart phone. This can be done so long as a user interface can be added to the device. A simple three segment numerical display can display the blood pressure value using, for example, monochrome or color segmented or matrix LCD, OLED, or other display technology like for example, E-ink displays. Segments can be designed or graphics on a matrix display can be designed to tell the user if they need to press harder or lighter. The advantage of a simple segmented display is that they are often lower cost and potentially lower power whereas a matrix display is generally more flexible as symbols can be changed by changing software. Color can also be used to give guidance to the user. Examples of such displays are presented in FIGS. 46A-46D. FIG. 46A illustrates three monochrome display examples showing appropriate pressure being applied. FIG. 46B illustrates three monochrome display examples showing slightly more pressure is needed. FIG. 46C illustrates three monochrome display examples showing much less pressure is needed. FIG. 46D illustrates three monochrome display examples showing that much more pressure is needed.

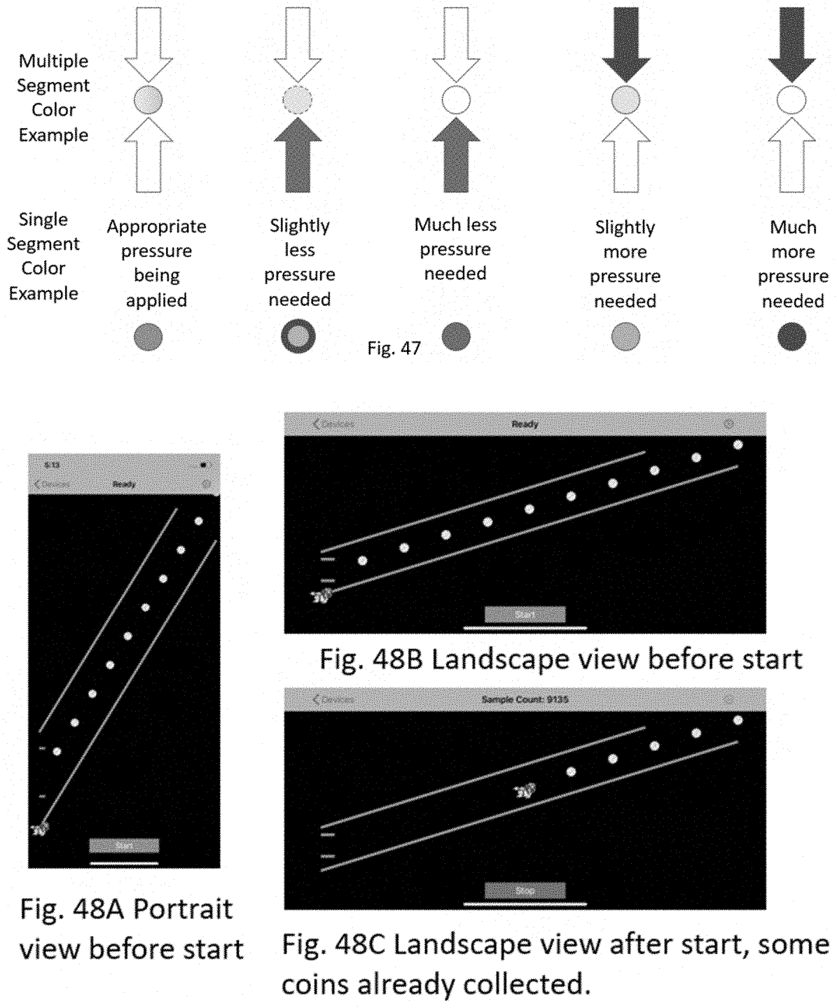

FIG. 47 illustrates how adding color, pattern, gradient or varied line weight can be used to produce a simple and compact display to tell the user how much pressure to apply. Color can be used to enhance communication to any of the monochrome display options as well as to even a single display LED. If desired, sound can be used independently or in addition to visual cues to guide the user. For example, a tone can be used to give feedback. A higher pitch can tell the user to push harder and a lower pitch can tell the user to push less hard (or vice versa). Two tones can be generated. Or, a varying tone can be generated to guide a user in the desired direction. For example, a higher pitch (or spacing of beeps) can tell a user that they are complying or getting “warm”, and a lower pitch or spacing of beeps can tell a user they are not complying or getting “cold”, so to speak. One can be controlled by the pressure and the other swept to generate a pressure profile and the user can be instructed to match the tones.

Gamification of the user interface can also be advantageous to make the act of blood pressure monitoring more fun, which is very likely to enhance user compliance. An illustrative example of a game user interface that can guide a user to apply the correct pressure profile is shown in FIGS. 48A—48C. Here, the applied pressure on the sensor controls the height of flying cartoon that is flying from left to right across the display. If the user follows the desired pressure profile, he/she will capture more coins and score more points. Thus, in FIG. 48, the subject is guided to follow the slope by their finger pressure which controls the height of the flying cartoon character or icon that captures coins as they follow the pressure slope. Screen shots from a prototype smartphone App are disclosed. FIG. 48A presents a portrait view before start, FIG. 48B illustrates a landscape view before start, and FIG. 48C illustrates a landscape view after start, wherein some coins are already collected.

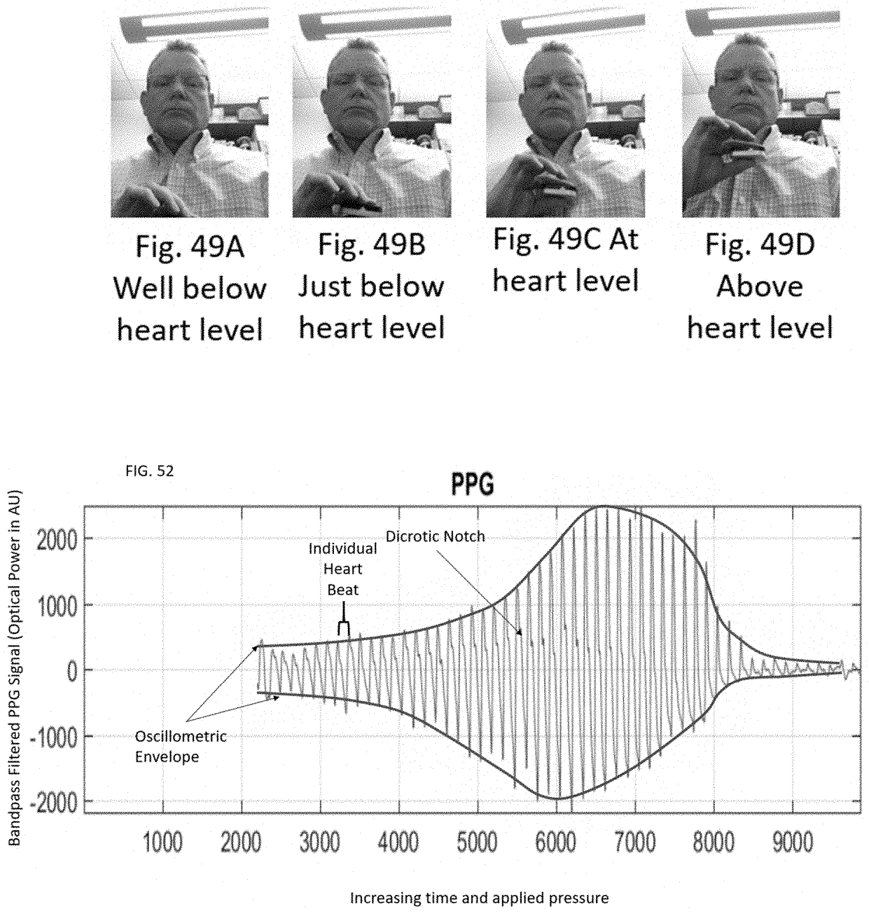

FIGS. 49A-49D illustrate aspects of estimating height of the BP sensor relative to heart level. The difference between arterial blood pressure at the heart and the blood pressure measured at a remote point in the arterial system varies linearly with the relative height difference between the remote point and the heart. This so-called hydrostatic difference is related to the relative density of blood and Hg and is approximately described by the simple physics of hydrostatics. The “gold standard” of noninvasive BP monitoring is auscultation by a trained observer using a pressure cuff on the brachial artery on the upper arm. The upper arm is convenient because on a sitting or supine subject it is relatively easy to keep the point of measurement close to the level of the heart. The fingertip by comparison can easily be at a significantly different level than the heart. One can instruct the user through various means including training, as well as printed and digital instructions, to practice placement of the finger at or near heart level. However, it is advantageous if the device/system can keep track of the relative height of the sensor. If the device/system knows the relative height of the sensor, it can make corrections mathematically to compensate for this height difference or instruct the user to put the device at heart level. This can lead to more accurate BP measurements.

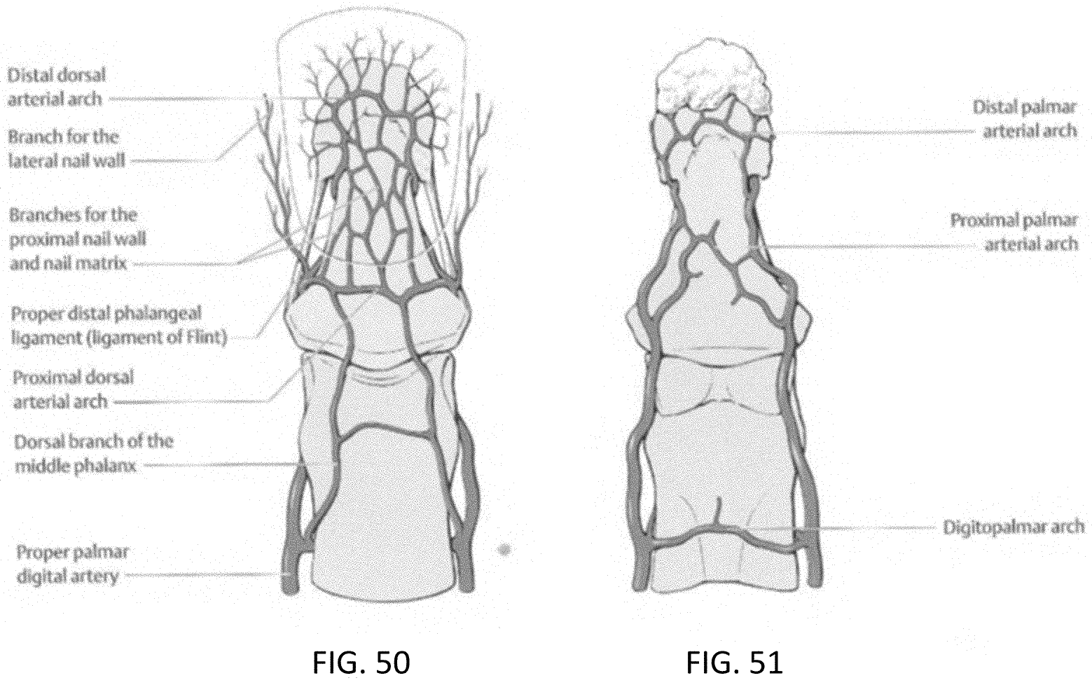

A further approach, aspects of which are presented in FIGS. 49A-49D, is to use a smart phone and sensors to determine the height of the device relative to the heart. In a configuration that uses a smart phone display as an integral part of the user interface this is particularly convenient. The subject can typically hold the device in one hand with a finger applying pressure on the device for measurement while holding the smart phone in the other hand (as illustrated) with the display facing them. The front facing camera can then be used take a picture that includes the device and the persons head and upper torso. The camera can focus distance, inclination, and the picture can be used to determine the distance from the heart to the device. Algorithms can be used to measure distance. It may be difficult to find the heart level on the picture so the device may use a more recognizable feature like the nose, mouth, or eyes and extrapolate that to the heart level based on an earlier calibration measurement or algorithmic compensation based on average anatomical ratios. An alignment(s) mark or other visual indicia can be placed on the handheld device as well to facilitate its detection by the camera of the smart phone to increase the robustness of the image processing. Certain newer BLE 5.0 protocols have the ability to measure orientation and position between two devices. This can be used in addition to the camera to increase the robustness of the relative position of the two devices. It will be appreciated by those of skill in the art that embodiments of the disclosure can include measuring the finger contact area and using this value to calibrate the pressure measurement from the pressure or force sensor, as appropriate. FIGS. 50-51 illustrate Dorsal and Palmar aspects of the arterial supply to the distal phalanx and nail structures, respectively.

### Exploiting Tissue Propagation and Wavelength and Spatial Diversity

It can be advantageous to use multiple wavelengths and multiple emitter/detector pairs to improve PPG versus pressure prediction of blood pressure and other cardiac parameters. One way to implement this is to exploit tissue scattering and absorption characteristics to better isolate an arterial PPG signal. This can be achieved by using both multiple wavelengths, multiple emitter/detector distances, or both.

In addition to pulsatile components from arteries, skin based PPG oscillometric measurements can be affected by the interaction of light with pulsatile blood outside of arteries, such as capillaries and arterioles, and also with venous blood, but likely to a lesser extent. Although the pulsatile component of the PPG signal is larger in arteries than in arterioles and capillaries, arterioles and capillaries are closer to the skin surface. This is generally true of skin around the body, and particularly true for the pad of the fingertip. In the layout of the circulatory system, the arteries in the finger are closer in diameter and closer in distance to the brachial artery in the upper arm than the capillaries and arterioles in the finger. Therefore, the fingertip artery can be expected to correlate more closely to the brachial pressure than fingertip arterioles and capillaries. For example, capillaries can be expected to collapse with a few tens of mmHg of applied surface pressure, as contrasted to arteries, which can be expected to require more than 100 mmHg surface pressure to collapse and cease pulsing. Applicants appreciate that it can be advantageous to measure only the PPG signal from the deeper arteries when attempting to accurately correlate those measurements to brachial blood pressures. However, it is difficult to do this on the skin because the arteries are farther from the skin surface.

Wavelengths in the near infrared penetrate tissue reasonably well because they scatter less than shorter wavelengths and because attenuation is generally lower. In general, wavelengths in the 660-1300 range, and more preferably the 800-1100 nm range, are used. Longer wavelengths generally travel farther in skin tissue. Beyond that wavelength range, water absorption becomes appreciable. Moreover, 1100 nm can be considered to be an upper limit in which silicon photodiodes are utilized, as wavelengths greater than 1100 nm are not generally detectable by such photodiodes. Below the lower portion of the above ranges (660-800 nm), scattering increases (increasing the effective photon path) and absorption from skin pigments and other chromophores in tissue also increase. This wavelength dependence can be exploited for improving the capture of arterial PPG by using multiple wavelengths, for example, at two spaced apart wavelengths at 940 nm 500 nm in one example. The 940 nm light can be expected to propagate deeply and interrogate the artery deeper in the tissue while also partially sampling the intervening arterioles and capillaries. The shorter wavelength light will propagate less and will mostly interrogate the shallower capillaries and arterioles. If one then normalizes or adjusts the magnitudes or amplitudes of the pulsatile component of the waveforms (e.g., from a scale of −1 to +1, or from 0 to +1) and then subtracts the resulting normalized 500 nm PPG waveform from the 940 nm PPG waveform, it is then possible to at least partially remove the unwanted contribution from the capillaries and arterioles. Such a scaled or normalized subtraction can be improved by using optical propagation models and machine learning optimization to pick the proper combination of the two wavelength components for an improved scaling and improved prediction of the arterial signal, by removing the effect of the near surface capillaries and arterioles. In addition, more than two wavelengths can be used in this manner to further improve the prediction.

The multiple scattering nature of light propagation in the near infrared wavelengths is another way to gain depth resolution and compensate for the unwanted contribution of capillary and arteriole pulsatile signal. Taking again for example light at 940 nm which propagates comparably well in tissue, you can analyze signals from different detector/emitter separation distances. Because of the slightly anisotropic and multiple scattering that takes place, the signal propagating between the detector and emitter combinations that are farther apart will have a larger component of signal from the deeper artery than the closer spaced emitter and detectors. These two different signals can be analyzed as described previously for the multiple wavelength data described above. Similarly, one can use two or more wavelength separation distances. Indeed, one can combine both multiple wavelengths and multiple positions to further enhance the isolation of the arterial component. A suitable range of target wavelengths for shallow transmission to detect the effect of capillaries or arterioles can be about 400 nm to about 780 nm, or any wavelength therebetween in increments of one nanometer, inclusive of the endpoints of that range. A suitable range of target wavelengths for deeper transmission to detect the effect of arteries can be about 660 nm to about 1300 nm, or any wavelength therebetween in increments of one nanometer, inclusive of the endpoints of that range. Preferably, the target wavelengths for the shallow and deeper transmission are separated by 250-500 nm, or any increment therebetween in increments of one nanometer, inclusive of the endpoints of that range.

The following references are germane to the aforementioned discussion and are incorporated by reference herein for all purposes: JING LIU, BRYAN PING-YEN YAN, WEN-XUAN DAI, XIAO-RONG DING, YUAN-TING ZHANG,1,3,4 AND NI ZHAO, “Multi-wavelength photoplethysmography method for skin arterial pulse extraction,” BIOMEDICAL OPTICS EXPRESS Vol. 7, No. 10, 1. Mendelson, Y. and Ochs, B, “Noninvasive pulse Oximetry Utilizing skin reflectance photoplethysmography,” IEEE TRANSACTIONS ON BIOMEDICAL ENGINEERING, VOL. 35, NO. 10, October 1988.

### Per Beat Analysis of PPG Waveforms Under Time Varying Applied Pressure

Other per-beat variations in the PPG waveform may be examined in accordance with the present disclosure as a function of applied pressure. This implementation goes beyond the standard oscillometric analysis which focuses on the change in PPG peak-to-peak value versus applied pressure. In this implementation, the change in the shape of the entire PPG beat can be analyzed versus pressure. This can provide additional information that can be used to improve predictions made by traditional oscillometric analysis or provide independent information about other cardio parameters including, but not limited to arterial stiffness, cardiac output, and the like. Further, this also goes beyond the existing approaches to estimation of cardiovascular parameters based on PPG waveform shape, as those methods do not look at the (change in) wave shape vs. applied pressure.

As can be seen in FIG. 52, there is variation in the shape of the beat in time as the applied pressure varies. A shift in the position of the systolic peak within the beat is visible. There also appears to be a time shift in the position of the dicrotic notch. One approach for dealing with these apparent changes PPG beat shape is to identify beats in the PPG waveform and then parameterize the per-beat shape parameters. One example of an approach for this can include identifying an average beat shape and then observing at the changes in each beat shape in time versus applied pressure.

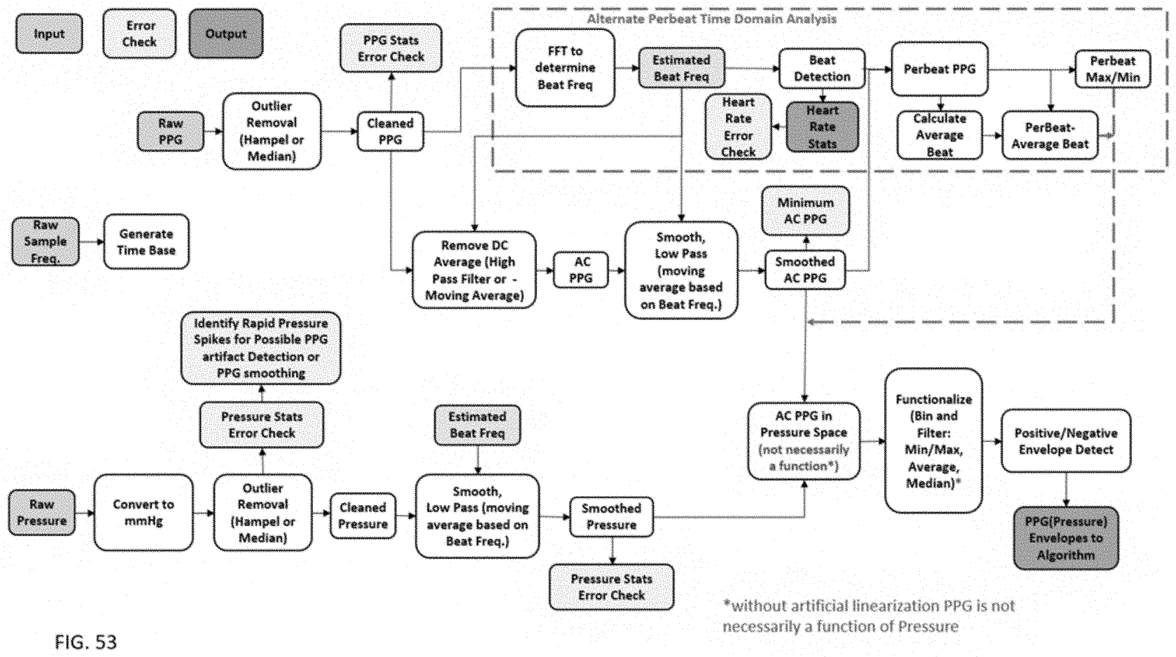

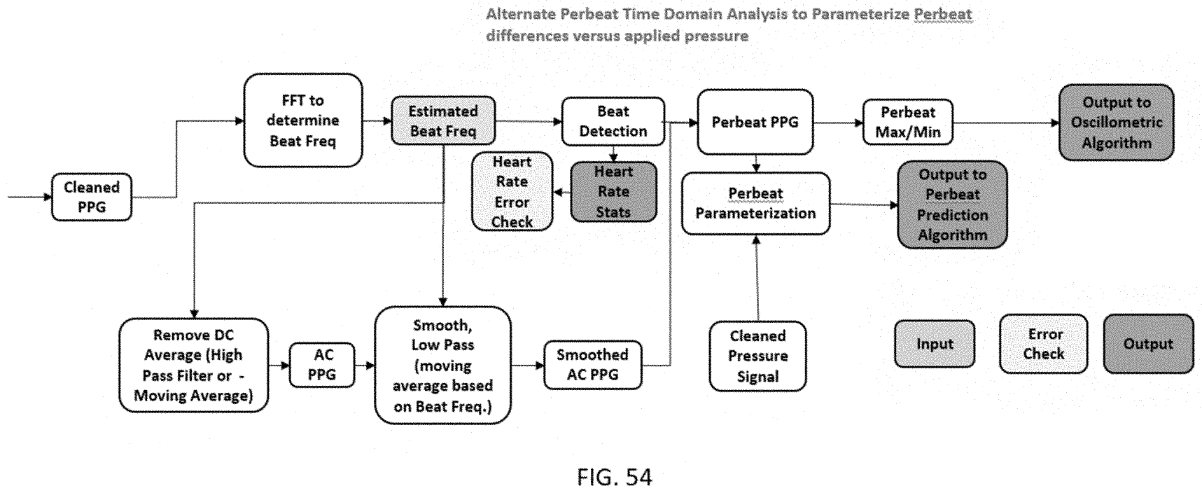

An illustrative process for carrying this out is illustrated in the flow charts of FIGS. 53 and 54. Given that PPG peak amplitude is already captured in a more standard PPG based oscillometric analysis, it can be advantageous to normalize the amplitude of each peak. Such normalization can be achieved, for example, by employing a linear transform function that maps the beat minima to 0, and the maxima to 1. If desired, this can be done on each beat separately, so that after this operation, all beats will span a range of 1.

Once parameterized, machine learning algorithms can be applied and/or linear and nonlinear optimization can be performed to relate these parameters to one or more reference cardio-parameters, including but not limited to blood pressure. Once a model is established, then this analysis can be used to predict a cardio-vascular parameter such as blood pressure. The beat data can be used independently to compute the prediction, or it can be used in combination with other measurements or analyses to make improved predictions. In the example of blood pressure, the per beat versus pressure analysis can be used to predict blood pressure or can be used in combination with a purely oscillometric based prediction. Further information from other sources can also be included such as ECG signals, differential PPG time of flight, pulse oximetry, skin temperature, and skin resistance to further improve the estimation of blood pressure or other cardio-parameters.

FIG. 52 illustrates PPG pulses in time with a constantly increasing external pressure ramp from approximately 50 to 140 mmHg. On the horizontal axis, one unit is equal to five milliseconds. FIG. 53 presents a flow chart for simultaneous oscillometric and per beat preprocessing and parameterization for constructing an algorithm to predict blood pressure and other cardiac parameters from a measured PPG signal under a known pressure profile. FIG. 54 presents a flow chart showing generalized Per Beat Preprocessing and Parameterization for constructing an algorithm to predict blood pressure and other cardiac parameters from a measured PPG signal under a known pressure profile.

The per beat time domain analysis for parameterization of the changes in each beat as a function of applied pressure is shown generalized in FIG. 54. The parametrization involves quantifying the difference in the shape of each beat as the applied pressure during that beat varies. One approach, as depicted in FIG. 53, can be to calculate an average beat and then parameterize the difference between an average beat and the beat at a given applied pressure. In another implementation it is possible to calculate the difference between each beat at a given pressure to the average beat at a fixed pressure. In either example, it is possible to normalize the amplitudes of the beats as described above in order to concentrate on per beat shape differences. Other forms of parameterization can include comparing differences in the per beat first, second, and/or nth order derivative. Likewise, the specific relative timing of beat morphological structures like the shape and time position of the dicrotic notch can also be a parameterized versus applied pressure. The skewness of the pulse shape is another shape parameter that can be used, among others such as kurtosis.

In further accordance with the disclosure, implementations of cuffless blood pressure measurement using a wrist mounted device, such as a watch, are provided.

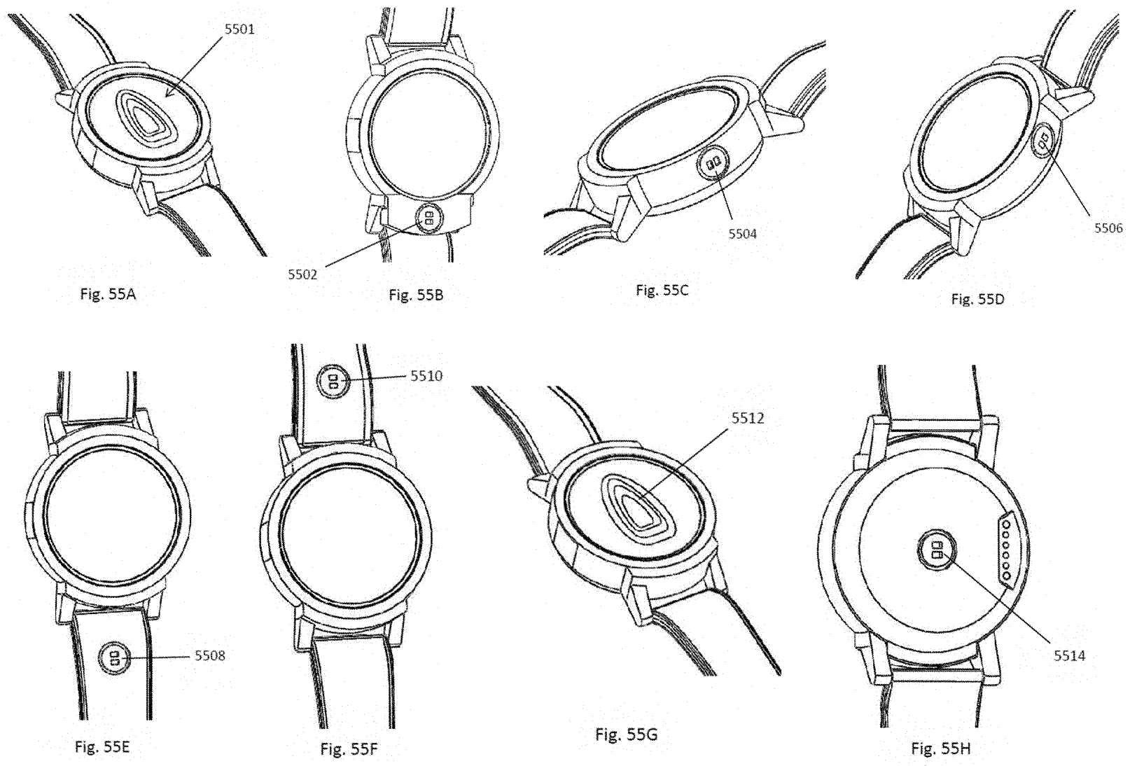

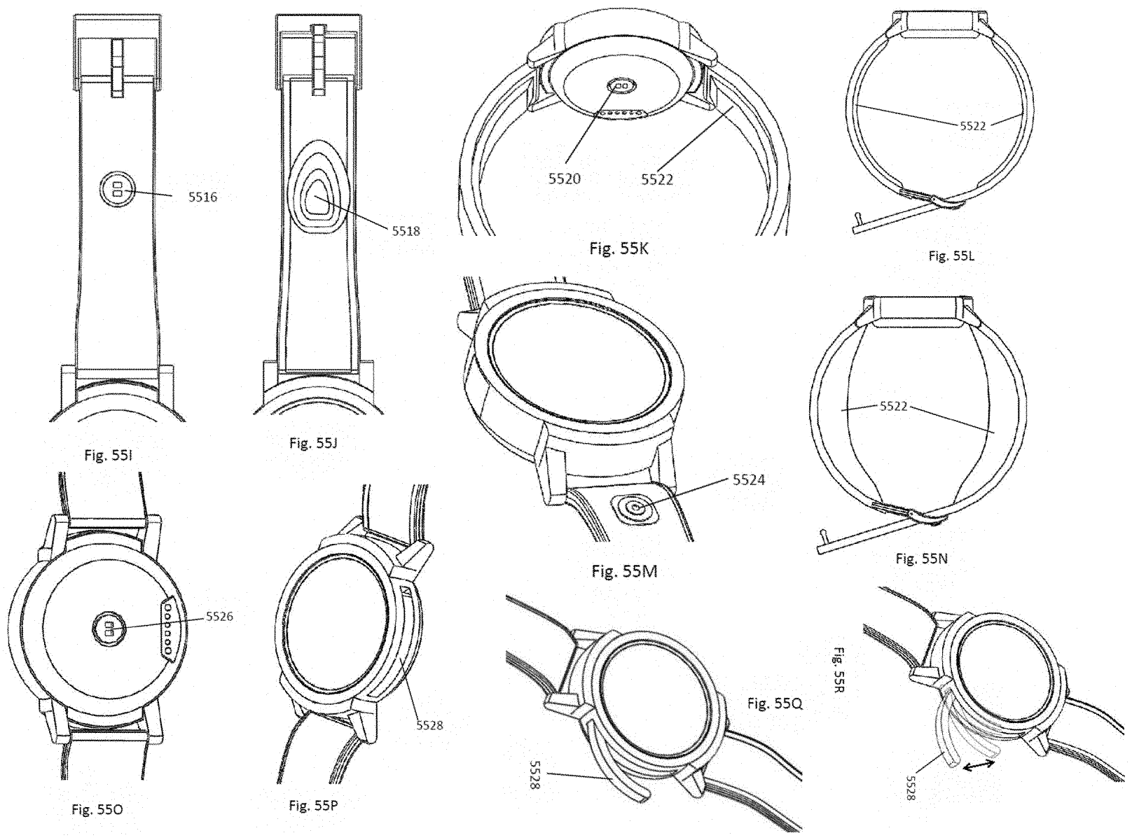

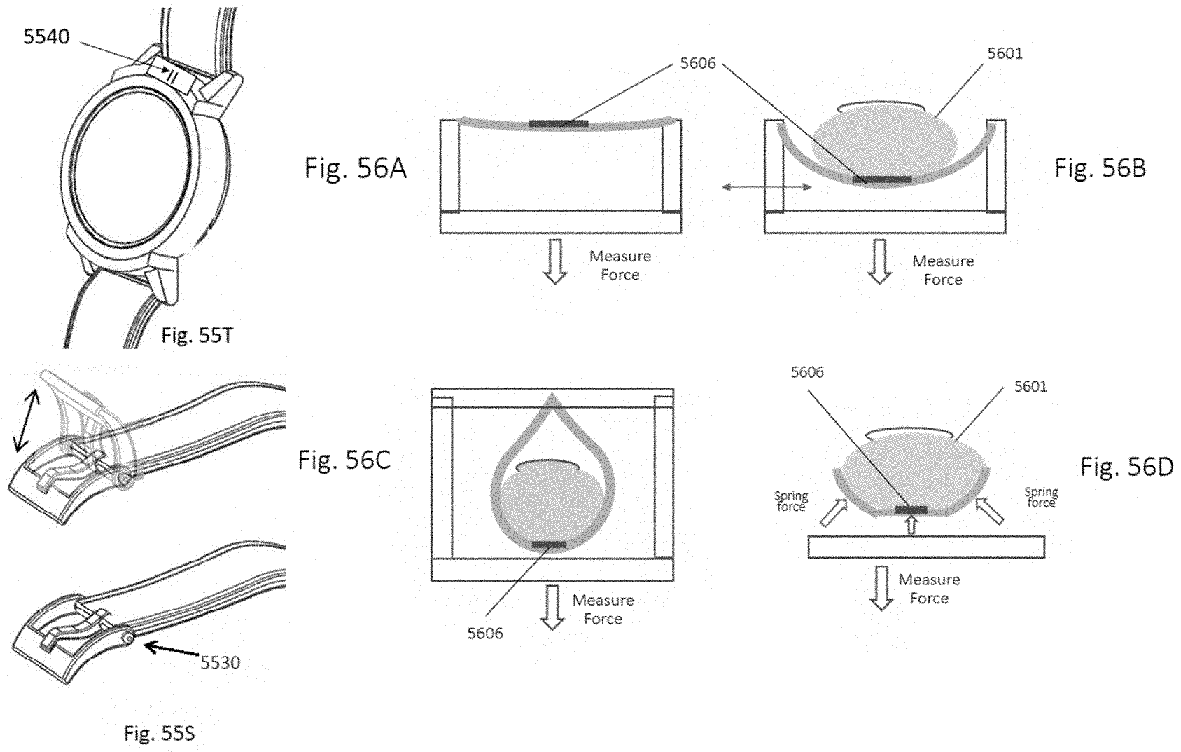

For purposes of illustration, and not limitation, FIGS. 55A-55T present aspects of a watch device that can be used to measure blood pressure. With suitable sensors built into a watch, the power supply and microprocessor support for the user interface (UI) can be provided directly by the watch. Communication can be wired or wireless, but because it is integrated, wired is believed to be the most convenient. The watch provides the UI or can connect to a smart device like phone or tablet to provide the user interface.

With watchband integration the sensor and electronics can be totally built into the band including power supply (battery) and signal processing and microcontroller. It can be configured as a clip-on to a watch band or made integral with the watch band. This can allow the volume of the electronics to be spread out over the watchband. This can then be sold with a watch or as a replacement band to fit smart watches. The system can be programmed to communicate with the watch for UI purposes using either a wired link or a wireless link. Example wireless technologies for wireless can be Bluetooth, Bluetooth low energy, and ultrasonic. This same communication can be used to communicate directly with a smart phone or tablet.

FIG. 55A illustrates a first implementation of a watch device in accordance with the disclosure that includes a sensor built into an LED display on the face of the watch, wherein the “fingerprint target” 5501 for placing a user's finger is displayed. FIG. 55B depicts an implementation that includes one or more sensors 5502 disposed on a rim of the watch face. FIG. 55C illustrates an implementation that includes one or more sensors 5504 disposed on a side of the watch face aligned with a center of the side of the watch face. FIG. 55D illustrates an implementation that includes one or more sensors 5506 disposed on a side of the watch face out of alignment with a center of the side of the watch face. FIG. 55E illustrates an implementation that includes one or more sensors 5508 built into a watch band segment that does not include a buckle. FIG. 55F illustrates an implementation that includes one or more sensors 5510 built into a watch band segment that does include a buckle. FIG. 55G illustrates a manner in which a LED watch display can display a target 5512 for a user to press their finger. FIG. 55H illustrates a sensor 5514 that is built into an underside of the watch face that compresses against a user's wrist to accomplish measurement of BP. FIG. 55I illustrates a sensor 5516 located on an underside of a watch band. FIG. 55J illustrates a pressure target 5518 located on an upper side of the band showing finger placement to permit sensor 5516 to complete a measurement. FIG. 55K depicts a sensor 5520 located on an underside of the watch body, and an uninflated bladder 5522 disposed along an inner edge of the watch band. FIG. 55L depicts the bladder 5522 uninflated, and a pump located in tail of the band. FIG. 55M shows an alternative arrangement wherein a pump actuator 5524 is disposed in an end of the band near the watch body.

FIG. 55N illustrates the bladder 5522 in an inflated condition to urge the bottom of the watch body against a user's skin to measure blood pressure. FIG. 55O illustrates sensor 5526 built into an underside of the watch face to be compressed against a user's wrist for detection and readout. FIG. 55P illustrates an actuator, such as a hinged ratcheted handle, that nests inside a side face of the watch body. The actuator can be reciprocated back and forth to inflate the bladder, if desired. FIG. 55Q illustrates the arm deployed so as to permit inflation of a bladder (e.g., 5522) to commence. FIG. 55R illustrates that actuation of the actuator can pump air into the bladder 5522 or to mechanically tighten the strap to facilitate taking the measurement. Alternatively, and with reference to FIG. 55S, the watch buckle can serve as a ratchet arm, with a button on the buckle to release pressure when the reading is complete. Thus, it will be appreciated that the PPG/pressure sensor can be placed in a variety of locations of the watch body or the wristband.

In some aspects of the disclosure, implementations are provided that conform closely to a user's finger shape in order to create a pressure around the artery that closely tracks the surface pressure that is actually measured. In some implementations, this can be achieved by using a finger cuff to apply pressure to a PPG sensor. This can be done in other ways, as discussed below. Many of these implementations for conforming to a user's finger can work both with Si micromachined pressure sensors as well as with a force/area approach. In these implementations, a user can apply very even pressure, and the external pressure can be expected to map well to the internal pressure on the artery. It will be appreciated that it can be helpful to account for finger size differences in determining pressure. A wider finger can give a larger force for the same pressure. It is possible to derive pressure from a force measurement, particularly when using an array pressure sensor to determine the finger contact area and/or measure the average pressure. But, if accommodations are made to adapt to the shape of the finger, this can create complications for determining pressure from a measured force if the area is not known. In the case of the hammock version on the left above, it is possible to make the vertical supports adjustable in height and/or orientation.

FIGS. 56A-B illustrate a sling or hammock option to accommodate a finger, wherein a user's finger is viewed from the end in FIG. 56B. As illustrated in FIG. 56A, a flexible web or a hammock including PPG sensor 5606 thereon is presented, wherein the overall assembly including the frame holding the hammock is disposed on a transducer or other element to measure the applied force to the system by a user's applied finger 5601. As is evident, the flexible nature of the hammock or web confirms to the user's finger and allows for variation of width from one user to another. FIG. 56C presents an embodiment where a hammock or web is suspended from an upper end to permit the web to wrap around a larger extent of a user's finger. FIG. 56D presents an implementation wherein a central platform including a PPG sensor 5606 is coupled to two resilient curved side panels that urge against a user's finger 5601 that is pressed into the device. The resilient but flexible side panels help distribute the stress evenly about the finger near the artery of interest.

The prototype embodiment 200 described herein above can be modified to incorporate a flexible or adjustable structure as illustrated in FIGS. 56A-56D to accommodate for different finger sizes. Alternatively, the device 200 can be provided with a kit of inserts of different sizes to accommodate different finger geometries. This permits a purchasing consumer, or a user generally, to insert a suitable insert into the device 200 to provide more reliable BP data. If desired, a user can have their finger measured and/or tested at a central location, such as a clinic for fit and function and be prescribed a suitable insert, or be provided with a custom manufactured insert based on the shape and/or underlying structure of their finger.

FIGS. 57A-57G present additional illustrative embodiments that include a deformable gel or liquid filled flexible bladder to provide an interface that wraps around or otherwise conforms to a user's finger, similar in some respects to the embodiments of FIGS. 12A-12B, that lack a rigid frame, and to FIGS. 27-37, which tend to include a rigid frame 5762 surrounding a flexible material. Such embodiments can be expected to provide a more uniform pressure on the finger and also provide more consistent tracking of applied surface pressure to the pressure at the artery of interest in the finger. A rigid container or housing can be provided that holds the low viscosity gel or flexible membrane filled with liquid, as described with respect to previous embodiments.

The embodiments of FIGS. 57A-57C are trough shaped to conform to the shape of the finger while the embodiments of FIGS. 57D-57G have a compound saddle profile that raise the sides only in the middle with a trough with the finger to facilitate contact with the PPG sensor 5706. Each device includes a perimeter frame 5762 and is filled with a gel material or a liquid filled bladder, and includes a PPG sensor on top. As such, from the side, a hump is visible in FIG. 57D, whereas from the end, there is a rectangular profile. In top plan view, the device can be elliptical (FIG. 57F) or oval (FIG. 57G) in shape. These devices can be used with a pressure sensor imbedded in a low viscosity gel or liquid. The devices can also be used in a force measurement mode by measuring force down on the whole sensor. Preferably the majority of the surface area of the sensor that is deflectable is in direct contact with the user's finger.

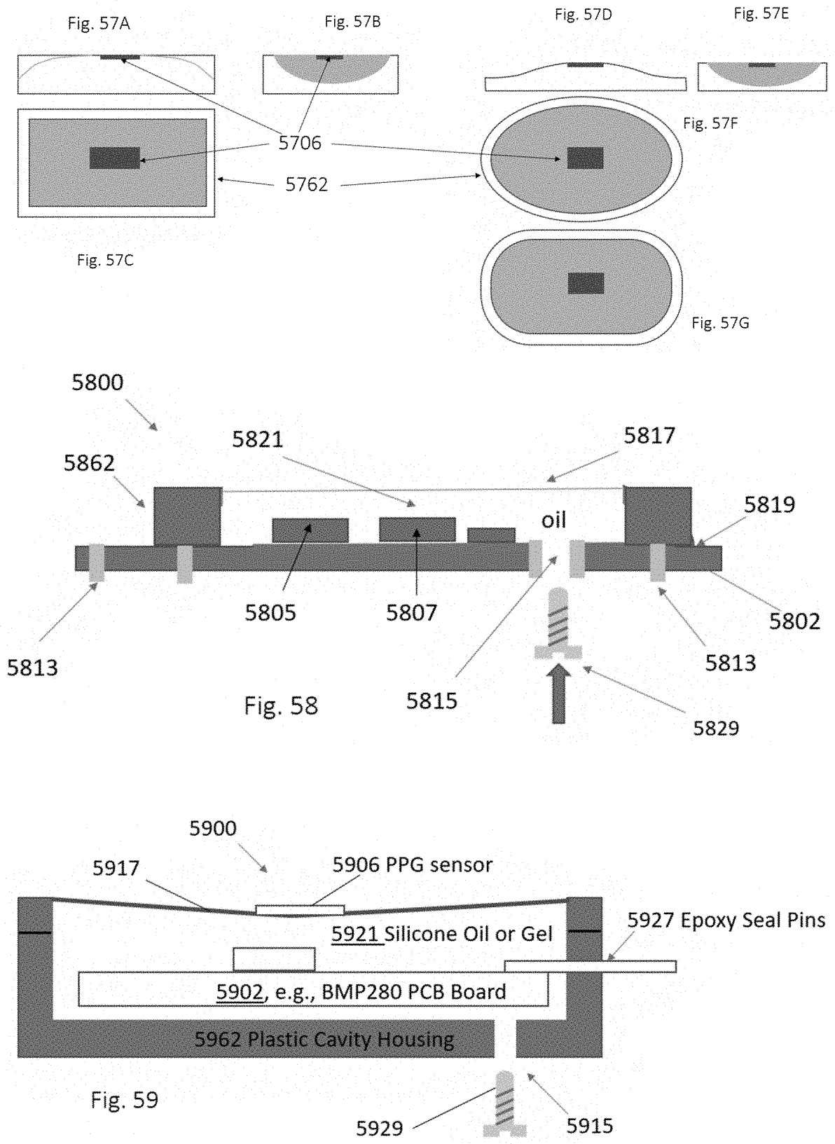

FIG. 58 presents a further implementation of a sensor package 5800 wherein a housing 5862 surrounds and defines a chamber that is filled with silicone oil or gel 5821. The housing 5862 is provided with a vent 5815 to permit introduction of fluids, such as the silicone oil or gel, and to permit evacuation of gases or other impurities. A set screw 5829 can be used to occlude the vent 8915. A PC board 5802 can be used. The board 5902 can be provided with a pressure sensor 5805 and an optical sensor 5807. Electrical interface pins 5813 provide an electrical connection between the PCB 5802 and a further device, or receptacle. A flexible membrane 5817 can be provided that is held in place with epoxy or other suitable adhesive. An adhesive seal 5819 can help seal the board 5802 to the frame 5862.

FIG. 59 presents a further implementation of a sensor package 5900 wherein a housing 5962 surrounds and defines a chamber that is filled with silicone oil or gel 5921. The housing 5962 is provided with a vent 5915 to permit introduction of fluids, such as the silicone oil or gel, and to permit evacuation of gases or other impurities. A set screw 5929 can be used to occlude the vent 5915. A PC board 5902 can be used. The board 5902 is held in place in the chamber by one or more seal pins, and the board can also be held in place by epoxy or other sealant material. A PPG sensor 5906 can be disposed on a flexible membrane 5917, and be in electrical communication with the board 5902. The membrane can be held in place with epoxy or other suitable adhesive.

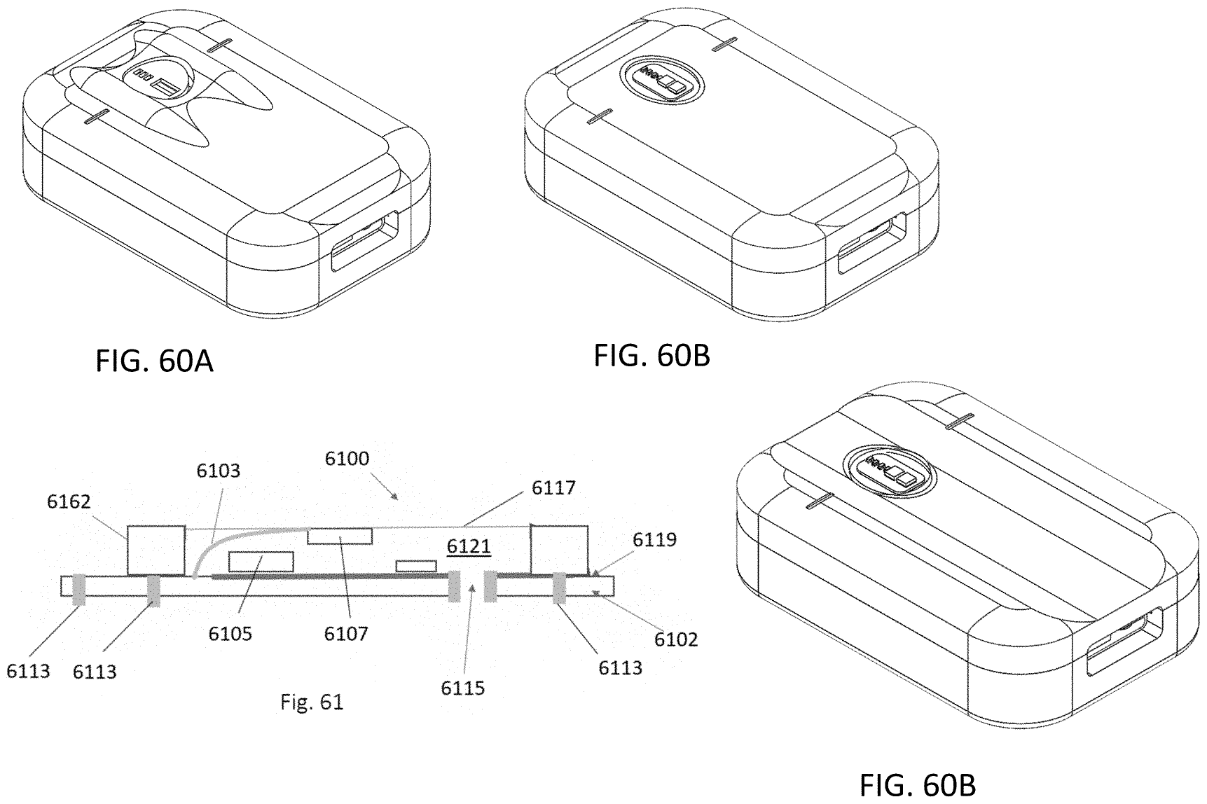

FIGS. 60A-60C present implementations of different finger interfaces for a blood pressure measurement system in accordance with the present disclosure.

FIG. 60A depicts a combination of a curved (e.g., plastic) housing and curved button that can be used to create a shape that will more accurately fit the curved profile of a finger. These two components can sit flush together so that the finger can rest in the resulting valley while pressing on the sensor. This geometry can help to spread the pressure evenly over the finger. Multiple sizes can be manufactured to accommodate various finger sizes and shapes.

FIG. 60B illustrates an embodiment of a device with a gel membrane with an embedded sensor can be used to evenly distribute pressure on the finger while taking a BP measurement. The purpose of this membrane is to provide a pressing area that will mold to the finger as pressure is applied. The pressure can be expected to become evenly distributed over the finger as the gel continues to mold to the finger shape with the applied pressure.

FIG. 60C presents an additional configuration that includes a gel membrane and sensor combination that features a trough in the housing. This trough can guide the finger to be aligned with the center of the device while the gel membrane ensures that the proper amount of pressure is applied to the finger.

FIG. 61 presents a further embodiment of a pressure and optical sensor package 6100 that includes a peripheral rigid frame 6162 attached to a PCB 6102 that includes a pressure sensor 6105 mounted thereon. An optical PPG chip 6107 is suspended above the PCT 6102 by a flex circuit 6103. The frame 6162 can be attached to the PCB 6102 by any suitable means, including for example solder, adhesive 6119, and/or sealant. The PCB 6102 further includes a plurality of electrical interface pins 6113 that are in electrical communication with various components of the package 6100. A port 6115 can be provided to fill the compartment under the membrane 6117 with optically clear oil or other liquid or gel material 6121. The port 6115 can be sealed with a fastener, plug or adhesive material after filling is complete. The pressure sensor and other components can first be mounted on the underlying (preferably rigid) PCB 6102. The flexible circuit connector 6103 can be used to attach the optical sensor to the membrane. The attachment adhesive is preferably selected to not attenuate IR signal, to be suitable for repeated fatigue over time, wo withstand mechanical cycling, and to be compatible with prolonged exposure to fluid 6121. Suitable means should be selected to prevent detachment of the optical sensor from the membrane, such as by molding the optical sensor 6107 into the membrane 6117, or other suitable technique.

### Example—DIGI™ Data Information System and Controller

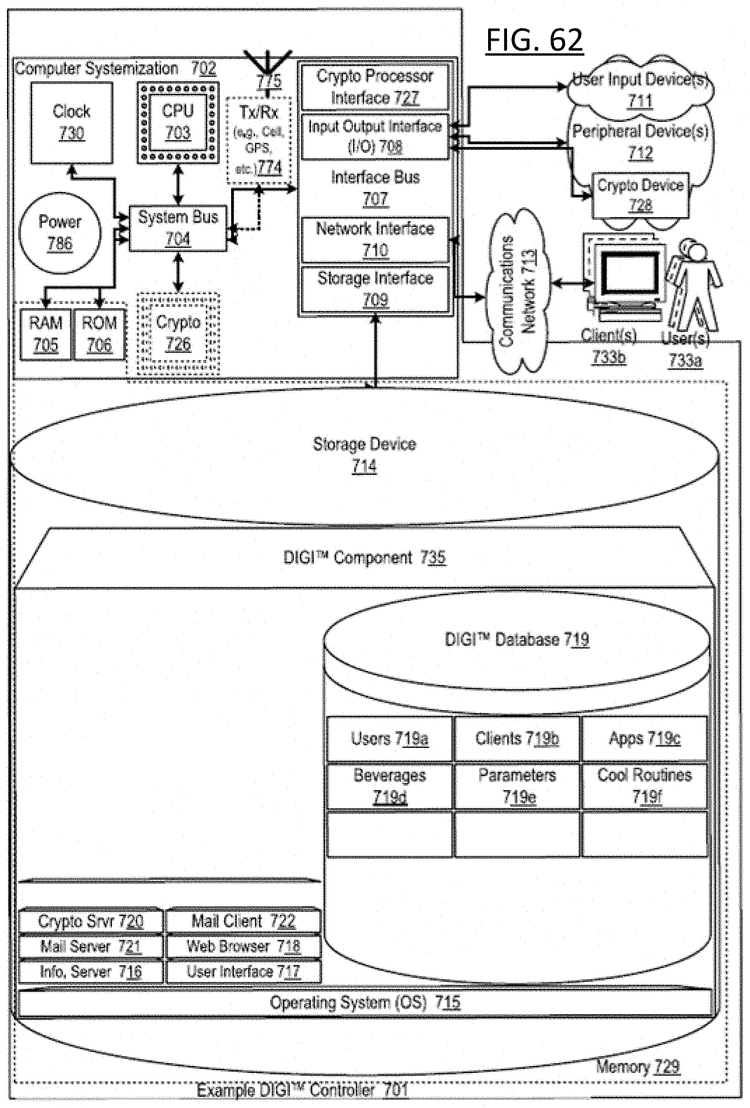

FIG. 62 illustrates inventive aspects of a DIGI™ controller 701 for controlling a system such as those described herein implementing some of the embodiments disclosed herein. In this embodiment, the DIGI™ controller 701 may serve to aggregate, process, store, search, serve, identify, instruct, generate, match, and/or facilitate interactions with a computer through various technologies, and/or other related data. In particular, it is contemplated that users of blood pressure monitoring devices as described herein can upload and back up their blood pressure data to a computer database system where the data can be stored retrieved and analyzed for various purposes, such as central monitoring, and conducting studies, provided that appropriate legal privacy requirements are met concerning medical data.

Typically, a user or users, e.g., 733a, which may be people or groups of users and/or other systems, may engage information technology systems (e.g., computers) to facilitate operation of the system and information processing. In turn, computers employ processors to process information; such processors 703 may be referred to as central processing units (CPU). One form of processor is referred to as a microprocessor. CPUs use communicative circuits to pass binary encoded signals acting as instructions to enable various operations. These instructions may be operational and/or data instructions containing and/or referencing other instructions and data in various processor accessible and operable areas of memory 729 (e.g., registers, cache memory, random access memory, etc.). Such communicative instructions may be stored and/or transmitted in batches (e.g., batches of instructions) as programs and/or data components to facilitate desired operations. These stored instruction codes, e.g., programs, may engage the CPU circuit components and other motherboard and/or system components to perform desired operations. One type of program is a computer operating system, which, may be executed by CPU on a computer; the operating system enables and facilitates users to access and operate computer information technology and resources. Some resources that may be employed in information technology systems include: input and output mechanisms through which data may pass into and out of a computer; memory storage into which data may be saved; and processors by which information may be processed. These information technology systems may be used to collect data for later retrieval, analysis, and manipulation, which may be facilitated through a database program. These information technology systems provide interfaces that allow users to access and operate various system components.

In one embodiment, the DIGI™ controller 701 may be connected to and/or communicate with entities such as, but not limited to: one or more users from user input devices 711; peripheral devices 712, user devices, servers; an optional cryptographic processor device 728; and/or a communications network 713. For example, the DIGI™ controller 701 may be connected to and/or communicate with users, e.g., 733a, operating client device(s), e.g., 733b, including, but not limited to, personal computer(s), server(s) and/or various mobile device(s) including, but not limited to, cellular telephone(s), smartphone(s) (e.g., iPhone®, Blackberry®, Android OS-based phones etc.), tablet computer(s) (e.g., Apple iPad™, HP Slate™, Motorola Xoom™, etc.), eBook reader(s) (e.g., Amazon Kindle™, Barnes and Noble's Nook™ eReader, etc.), laptop computer(s), notebook(s), netbook(s), gaming console(s) (e.g., XBOX Live™, Nintendo® DS, Sony PlayStation® Portable, etc.), portable scanner(s) and/or the like.

Networks are commonly thought to comprise the interconnection and interoperation of clients, servers, and intermediary nodes in a graph topology. It should be noted that the term “server” as used throughout this application refers generally to a computer, other device, program, or combination thereof that processes and responds to the requests of remote users across a communications network. Servers serve their information to requesting “clients.” The term “client” as used herein refers generally to a computer, program, other device, user and/or combination thereof that is capable of processing and making requests and obtaining and processing any responses from servers across a communications network. A computer, other device, program, or combination thereof that facilitates, processes information and requests, and/or furthers the passage of information from a source user to a destination user is commonly referred to as a “node.” Networks are generally thought to facilitate the transfer of information from source points to destinations. A node specifically tasked with furthering the passage of information from a source to a destination is commonly called a “router.” There are many forms of networks such as Local Area Networks (LANs), Pico networks, Wide Area Networks (WANs), Wireless Networks (WLANs), etc. For example, the Internet is generally accepted as being an interconnection of a multitude of networks whereby remote clients and servers may access and interoperate with one another.

The DIGI™ controller 701 may be based on computer systems that may comprise, but are not limited to, components such as: a computer systemization 702 connected to memory 729.

Computer Systemization

A computer systemization 702 may comprise a clock 730, central processing unit (“CPU(s)” and/or “processor(s)” (these terms are used interchangeable throughout the disclosure unless noted to the contrary)) 703, a memory 729 (e.g., a read only memory (ROM) 706, a random access memory (RAM) 705, etc.), and/or an interface bus 707, and most frequently, although not necessarily, are all interconnected and/or communicating through a system bus 704 on one or more (mother)board(s) 702 having conductive and/or otherwise transportive circuit pathways through which instructions (e.g., binary encoded signals) may travel to effect communications, operations, storage, etc. Optionally, the computer systemization may be connected to an internal power source 786; e.g., optionally the power source may be internal. Optionally, a cryptographic processor 726 and/or transceivers (e.g., ICs) 774 may be connected to the system bus. In another embodiment, the cryptographic processor and/or transceivers may be connected as either internal and/or external peripheral devices 712 via the interface bus I/O. In turn, the transceivers may be connected to antenna(s) 775, thereby effectuating wireless transmission and reception of various communication and/or sensor protocols; for example the antenna(s) may connect to: a Texas Instruments WiLink WL1283 transceiver chip (e.g., providing 802.11n, Bluetooth 3.0, FM, global positioning system (GPS) (thereby allowing DIGI™ controller to determine its location)); Broadcom BCM4329FKUBG transceiver chip (e.g., providing 802.11n, Bluetooth 2.1+EDR, FM, etc.); a Broadcom BCM4750IUB8 receiver chip (e.g., GPS); an Infineon Technologies X-Gold 618-PMB9800 (e.g., providing 2G/3G HSDPA/HSUPA communications); and/or the like. The system clock typically has a crystal oscillator and generates a base signal through the computer systemization's circuit pathways. The clock is typically coupled to the system bus and various clock multipliers that will increase or decrease the base operating frequency for other components interconnected in the computer systemization. The clock and various components in a computer systemization drive signals embodying information throughout the system. Such transmission and reception of instructions embodying information throughout a computer systemization may be commonly referred to as communications. These communicative instructions may further be transmitted, received, and the cause of return and/or reply communications beyond the instant computer systemization to: communications networks, input devices, other computer systemizations, peripheral devices, and/or the like. Of course, any of the above components may be connected directly to one another, connected to the CPU, and/or organized in numerous variations employed as exemplified by various computer systems.

The CPU comprises at least one high-speed data processor adequate to execute program components for executing user and/or system-generated requests. Often, the processors themselves will incorporate various specialized processing units, such as, but not limited to: integrated system (bus) controllers, memory management control units, floating point units, and even specialized processing sub-units like graphics processing units, digital signal processing units, and/or the like. Additionally, processors may include internal fast access addressable memory, and be capable of mapping and addressing memory 729 beyond the processor itself; internal memory may include, but is not limited to: fast registers, various levels of cache memory (e.g., level 1, 2, 3, etc.), RAM, etc. The processor may access this memory through the use of a memory address space that is accessible via instruction address, which the processor can construct and decode allowing it to access a circuit path to a specific memory address space having a memory state. The CPU may be a microprocessor such as: AMD's Athlon, Duron and/or Opteron; ABM's application, embedded and secure processors; IBM and/or Motorola's DragonBall and PowerPC; IBM's and Sony's Cell processor; Intel's Celeron, Core (2) Duo, Itanium, Pentium, Xeon, and/or XScale; and/or the like processor(s). The CPU interacts with memory through instruction passing through conductive and/or transportive conduits (e.g., (printed) electronic and/or optic circuits) to execute stored instructions (i.e., program code) according to conventional data processing techniques. Such instruction passing facilitates communication within the DIGI™ controller and beyond through various interfaces. Should processing requirements dictate a greater amount speed and/or capacity, distributed processors (e.g., Distributed DIGI™ embodiments), mainframe, multi-core, parallel, and/or super-computer architectures may similarly be employed. Alternatively, should deployment requirements dictate greater portability, smaller Personal Digital Assistants (PDAs) may be employed.

Depending on the particular implementation, features of the DIGI™ implementations may be achieved by implementing a microcontroller such as CAST's R8051XC2 microcontroller; Intel's MCS 51 (i.e., 8051 microcontroller); and/or the like. Also, to implement certain features of the DIGI™ embodiments, some feature implementations may rely on embedded components, such as: Application-Specific Integrated Circuit (“ASIC”), Digital Signal Processing (“DSP”), Field Programmable Gate Array (“FPGA”), and/or the like embedded technology. For example, any of the DIGI™ component collection (distributed or otherwise) and/or features may be implemented via the microprocessor and/or via embedded components; e.g., via ASIC, coprocessor, DSP, FPGA, and/or the like. Alternately, some implementations of the DIGI™ may be implemented with embedded components that are configured and used to achieve a variety of features or signal processing.

Depending on the particular implementation, the embedded components may include software solutions, hardware solutions, and/or some combination of both hardware/software solutions. For example, DIGI™ features discussed herein may be achieved through implementing FPGAs, which are semiconductor devices containing programmable logic components called “logic blocks”, and programmable interconnects, such as the high performance FPGA Virtex series and/or the low cost Spartan series manufactured by Xilinx. Logic blocks and interconnects can be programmed by the customer or designer, after the FPGA is manufactured, to implement any of the DIGI™ features. A hierarchy of programmable interconnects allow logic blocks to be interconnected as needed by the DIGI™ system designer/administrator, somewhat like a one-chip programmable breadboard. An FPGA's logic blocks can be programmed to perform the function of basic logic gates such as AND, and XOR, or more complex combinational functions such as decoders or simple mathematical functions. In most FPGAs, the logic blocks also include memory elements, which may be simple flip-flops or more complete blocks of memory. In some circumstances, the DIGI™ may be developed on regular FPGAs and then migrated into a fixed version that more resembles ASIC implementations. Alternate or coordinating implementations may migrate DIGI™ controller features to a final ASIC instead of or in addition to FPGAs. Depending on the implementation all of the aforementioned embedded components and microprocessors may be considered the “CPU” and/or “processor” for the DIGI™

### Power Source

The power source 786 may be of any standard form for powering small electronic circuit board devices such as the following power cells: alkaline, lithium hydride, lithium ion, lithium polymer, nickel cadmium, solar cells, and/or the like. Other types of AC or DC power sources may be used as well. In the case of solar cells, in one embodiment, the case provides an aperture through which the solar cell may capture photonic energy. The power cell 786 is connected to at least one of the interconnected subsequent components of the DIGI™ thereby providing an electric current to all subsequent components. In one example, the power source 786 is connected to the system bus component 704. In an alternative embodiment, an outside power source 786 is provided through a connection across the I/O 708 interface. For example, a USB and/or IEEE 1394 connection carries both data and power across the connection and is therefore a suitable source of power.

Interface Adapters

Interface bus(ses) 707 may accept, connect, and/or communicate to a number of interface adapters, conventionally although not necessarily in the form of adapter cards, such as but not limited to: input output interfaces (I/O) 708, storage interfaces 709, network interfaces 710, and/or the like. Optionally, cryptographic processor interfaces 727 similarly may be connected to the interface bus. The interface bus provides for the communications of interface adapters with one another as well as with other components of the computer systemization. Interface adapters are adapted for a compatible interface bus. Interface adapters conventionally connect to the interface bus via a slot architecture. Conventional slot architectures may be employed, such as, but not limited to: Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and/or the like.

Storage interfaces 709 may accept, communicate, and/or connect to a number of storage devices such as, but not limited to: storage devices 714, removable disc devices, and/or the like. Storage interfaces may employ connection protocols such as, but not limited to: (Ultra) (Serial) Advanced Technology Attachment (Packet Interface) ((Ultra) (Serial) ATA(PI)), (Enhanced) Integrated Drive Electronics ((E)IDE), Institute of Electrical and Electronics Engineers (IEEE) 1394, fiber channel, Small Computer Systems Interface (SCSI), Universal Serial Bus (USB), and/or the like.

Network interfaces 710 may accept, communicate, and/or connect to a communications network 713. Through a communications network 713, the DIGI™ controller is accessible through remote clients 733b (e.g., computers with web browsers) by users 733a. Network interfaces may employ connection protocols such as, but not limited to: direct connect, Ethernet (thick, thin, twisted pair 10/100/1000 Base T, and/or the like), Token Ring, wireless connection such as IEEE 802.11a-x, and/or the like. Should processing requirements dictate a greater amount speed and/or capacity, distributed network controllers (e.g., Distributed DIGI™), architectures may similarly be employed to pool, load balance, and/or otherwise increase the communicative bandwidth required by the DIGI™ controller. A communications network may be any one and/or the combination of the following: a direct interconnection; the Internet; a Local Area Network (LAN); a Metropolitan Area Network (MAN); an Operating Missions as Nodes on the Internet (OMNI); a secured custom connection; a Wide Area Network (WAN); a wireless network (e.g., employing protocols such as, but not limited to a Wireless Application Protocol (WAP), I-mode, and/or the like); and/or the like. A network interface may be regarded as a specialized form of an input output interface. Further, multiple network interfaces 710 may be used to engage with various communications network types 713. For example, multiple network interfaces may be employed to allow for the communication over broadcast, multicast, and/or unicast networks.

Input Output interfaces (I/O) 708 may accept, communicate, and/or connect to user input devices 711, peripheral devices 712, cryptographic processor devices 728, and/or the like. I/O may employ connection protocols such as, but not limited to: audio: analog, digital, monaural, RCA, stereo, and/or the like; data: Apple Desktop Bus (ADB), IEEE 1394a-b, serial, universal serial bus (USB); infrared; joystick; keyboard; midi; optical; PC AT; PS/2; parallel; radio; video interface: Apple Desktop Connector (ADC), BNC, coaxial, component, composite, digital, Digital Visual Interface (DVI), high-definition multimedia interface (HDMI), RCA, RF antennae, S-Video, VGA, and/or the like; wireless transceivers: 802.11a/b/g/n/x; Bluetooth; cellular (e.g., code division multiple access (CDMA), high speed packet access (HSPA(+)), high-speed downlink packet access (HSDPA), global system for mobile communications (GSM), long term evolution (LTE), WiMax, etc.); and/or the like. One typical output device may include a video display, which typically comprises a Cathode Ray Tube (CRT) or Liquid Crystal Display (LCD) based monitor with an interface (e.g., DVI circuitry and cable) that accepts signals from a video interface, may be used. The video interface composites information generated by a computer systemization and generates video signals based on the composited information in a video memory frame. Another output device is a television set, which accepts signals from a video interface. Typically, the video interface provides the composited video information through a video connection interface that accepts a video display interface (e.g., an RCA composite video connector accepting an RCA composite video cable; a DVI connector accepting a DVI display cable, etc.).

User input devices 711 often are a type of peripheral device 712 (see below) and may include: card readers, dongles, finger print readers, gloves, graphics tablets, joysticks, keyboards, microphones, mouse (mice), remote controls, retina readers, touch screens (e.g., capacitive, resistive, etc.), trackballs, trackpads, sensors (e.g., accelerometers, ambient light, GPS, gyroscopes, proximity, etc.), styluses, and/or the like.

Peripheral devices 712, such as user devices 100 or other components of the system, such as peripheral sensors and the like, may be connected and/or communicate to I/O and/or other facilities of the like such as network interfaces, storage interfaces, directly to the interface bus, system bus, the CPU, and/or the like. Peripheral devices may be external, internal and/or part of the DIGI™ controller. Cryptographic units such as, but not limited to, microcontrollers, processors 726, interfaces 727, and/or devices 728 may be attached, and/or communicate with the DIGI™ controller. A MC68HC16 microcontroller, manufactured by Motorola Inc., may be used for and/or within cryptographic units. The MC68HC16 microcontroller utilizes a 16-bit multiply-and-accumulate instruction in the 16 MHz configuration and requires less than one second to perform a 512-bit RSA private key operation. Cryptographic units support the authentication of communications from interacting agents, as well as allowing for anonymous transactions. Cryptographic units may also be configured as part of CPU. Equivalent microcontrollers and/or processors may also be used. Other commercially available specialized cryptographic processors include: the Broadcom's CryptoNetX and other Security Processors; nCipher's nShield, SafeNet's Luna PCI (e.g., 7100) series; Semaphore Communications' 40 MHz Roadrunner 184; Sun's Cryptographic Accelerators (e.g., Accelerator 6000 PCIe Board, Accelerator 500 Daughtercard); Via Nano Processor (e.g., L2100, L2200, U2400) line, which is capable of performing 500+MB/s of cryptographic instructions; VLSI Technology's 33 MHz 6868; and/or the like.

Memory Generally, any mechanization and/or embodiment allowing a processor to affect the storage and/or retrieval of information is regarded as memory 729. However, memory is a fungible technology and resource, thus, any number of memory embodiments may be employed in lieu of or in concert with one another. It is to be understood that the DIGI™ controller and/or a computer systemization may employ various forms of memory 729. For example, a computer systemization may be configured wherein the functionality of on-chip CPU memory (e.g., registers), RAM, ROM, and any other storage devices are provided by a paper punch tape or paper punch card mechanism; of course, such an embodiment would result in an extremely slow rate of operation. In a typical configuration, memory 729 will include ROM 706, RAM 705, and a storage device 714. A storage device 714 may be any conventional computer system storage. Storage devices may include a drum; a (fixed and/or removable) magnetic disk drive; a magneto-optical drive; an optical drive (i.e., Blueray, CD ROM/RAM/Recordable (R)/ReWritable (RW), DVD R/RW, HD DVD R/RW etc.); an array of devices (e.g., Redundant Array of Independent Disks (RAID)); solid state memory devices (USB memory, solid state drives (SSD), etc.); other processor-readable storage mediums; and/or other devices of the like. Thus, a computer systemization generally requires and makes use of memory.

Component Collection

The memory 729 may contain a collection of program and/or database components and/or data such as, but not limited to: operating system component(s) 715 (operating system); information server component(s) 716 (information server); user interface component(s) 717 (user interface); Web browser component(s) 718 (Web browser); database(s) 719; mail server component(s) 721; mail client component(s) 722; cryptographic server component(s) 720 (cryptographic server) and/or the like (i.e., collectively a component collection). These components may be stored and accessed from the storage devices and/or from storage devices accessible through an interface bus. Although non-conventional program components such as those in the component collection, typically, are stored in a local storage device 714, they may also be loaded and/or stored in memory such as: peripheral devices, RAM, remote storage facilities through a communications network, ROM, various forms of memory, and/or the like.

Operating System

The operating system component 715 is an executable program component facilitating the operation of the DIGI™ controller. Typically, the operating system facilitates access of I/O, network interfaces, peripheral devices, storage devices, and/or the like. The operating system may be a highly fault tolerant, scalable, and secure system such as: Apple Macintosh OS X (Server); AT&T Plan 9; Be OS; Unix and Unix-like system distributions (such as AT&T's UNIX; Berkley Software Distribution (BSD) variations such as FreeBSD, NetBSD, OpenBSD, and/or the like; Linux distributions such as Red Hat, Ubuntu, and/or the like); and/or the like operating systems. However, more limited and/or less secure operating systems also may be employed such as Apple Macintosh OS, IBM OS/2, Microsoft DOS, Microsoft Windows 2000/2003/3.1/95/98/CE/Millenium/NT/Vista/XP (Server), Palm OS, and/or the like. An operating system may communicate to and/or with other components in a component collection, including itself, and/or the like. Most frequently, the operating system communicates with other program components, user interfaces, and/or the like. For example, the operating system may contain, communicate, generate, obtain, and/or provide program component, system, user, and/or data communications, requests, and/or responses. The operating system, once executed by the CPU, may enable the interaction with communications networks, data, I/O, peripheral devices, program components, memory, user input devices, and/or the like. The operating system may provide communications protocols that allow the DIGI™ controller to communicate with other entities through a communications network 713. Various communication protocols may be used by the DIGI™ controller as a subcarrier transport mechanism for interaction, such as, but not limited to: multicast, TCP/IP, UDP, unicast, and/or the like.

Information Server

An information server component 716 is a stored program component that is executed by a CPU. The information server may be a conventional Internet information server such as, but not limited to Apache Software Foundation's Apache, Microsoft's Internet Information Server, and/or the like. The information server may allow for the execution of program components through facilities such as Active Server Page (ASP), ActiveX, (ANSI) (Objective-) C (++), C# and/or .NET, Common Gateway Interface (CGI) scripts, dynamic (D) hypertext markup language (HTML), FLASH, Java, JavaScript, Practical Extraction Report Language (PERL), Hypertext Pre-Processor (PHP), pipes, Python, wireless application protocol (WAP), WebObjects, and/or the like. The information server may support secure communications protocols such as, but not limited to, File Transfer Protocol (FTP); HyperText Transfer Protocol (HTTP); Secure Hypertext Transfer Protocol (HTTPS), Secure Socket Layer (SSL), messaging protocols (e.g., America Online (AOL) Instant Messenger (AIM), Application Exchange (APEX), ICQ, Internet Relay Chat (IRC), Microsoft Network (MSN) Messenger Service, Presence and Instant Messaging Protocol (PRIM), Internet Engineering Task Force's (IETF's) Session Initiation Protocol (SIP), SIP for Instant Messaging and Presence Leveraging Extensions (SIMPLE), open XML-based Extensible Messaging and Presence Protocol (XMPP) (i.e., Jabber or Open Mobile Alliance's (OMA's) Instant Messaging and Presence Service (IMPS)), Yahoo! Instant Messenger Service, and/or the like. The information server provides results in the form of Web pages to Web browsers, and allows for the manipulated generation of the Web pages through interaction with other program components. After a Domain Name System (DNS) resolution portion of an HTTP request is resolved to a particular information server, the information server resolves requests for information at specified locations on the DIGI™ controller based on the remainder of the HTTP request. For example, a request such as http://123.124.125.126/mylnformation.html might have the IP portion of the request “123.124.125.126” resolved by a DNS server to an information server at that IP address; that information server might in turn further parse the http request for the “/mylnformation.html” portion of the request and resolve it to a location in memory containing the information “mylnformation.html.” Additionally, other information serving protocols may be employed across various ports, e.g., FTP communications across port 21, and/or the like. An information server may communicate to and/or with other components in a component collection, including itself, and/or facilities of the like. Most frequently, the information server communicates with the DIGI™ database 719, operating systems, other program components, user interfaces, Web browsers, and/or the like.

Access to the DIGI™ database may be achieved through a number of database bridge mechanisms such as through scripting languages as enumerated below (e.g., CGI) and through inter-application communication channels as enumerated below (e.g., CORBA, WebObjects, etc.). Any data requests through a Web browser are parsed through the bridge mechanism into appropriate grammars as required by the DIGI™. In one embodiment, the information server would provide a Web form accessible by a Web browser. Entries made into supplied fields in the Web form are tagged as having been entered into the particular fields, and parsed as such. The entered terms are then passed along with the field tags, which act to instruct the parser to generate queries directed to appropriate tables and/or fields. In one embodiment, the parser may generate queries in standard SQL by instantiating a search string with the proper join/select commands based on the tagged text entries, wherein the resulting command is provided over the bridge mechanism to the DIGI™ as a query. Upon generating query results from the query, the results are passed over the bridge mechanism, and may be parsed for formatting and generation of a new results Web page by the bridge mechanism. Such a new results Web page is then provided to the information server, which may supply it to the requesting Web browser.

Also, an information server may contain, communicate, generate, obtain, and/or provide program component, system, user, and/or data communications, requests, and/or responses.

User Interface

Computer interfaces in some respects are similar to automobile operation interfaces. Automobile operation interface elements such as steering wheels, gearshifts, and speedometers facilitate the access, operation, and display of automobile resources, and status. Computer interaction interface elements such as check boxes, cursors, menus, scrollers, and windows (collectively and commonly referred to as widgets) similarly facilitate the access, capabilities, operation, and display of data and computer hardware and operating system resources, and status. Operation interfaces are commonly called user interfaces. Graphical user interfaces (GUIs) such as the Apple Macintosh Operating System's Aqua, IBM's OS/2, Microsoft's Windows 2000/2003/3.1/95/98/CE/Millenium/NT/XP/Vista/7 (i.e., Aero), Unix's X-Windows (e.g., which may include additional Unix graphic interface libraries and layers such as K Desktop Environment (KDE), mythTV and GNU Network Object Model Environment (GNOME)), web interface libraries (e.g., ActiveX, AJAX, (D)HTML, FLASH, Java, JavaScript, etc. interface libraries such as, but not limited to, Dojo, jQuery(UI), MooTools, Prototype, script.aculo.us, SWFObject, Yahoo! User Interface, any of which may be used and) provide a baseline and means of accessing and displaying information graphically to users.

A user interface component 717 is a stored program component that is executed by a CPU. The user interface may be a conventional graphic user interface as provided by, with, and/or atop operating systems and/or operating environments such as already discussed. The user interface may allow for the display, execution, interaction, manipulation, and/or operation of program components and/or system facilities through textual and/or graphical facilities. The user interface provides a facility through which users may affect, interact, and/or operate a computer system. A user interface may communicate to and/or with other components in a component collection, including itself, and/or facilities of the like. Most frequently, the user interface communicates with operating systems, other program components, and/or the like. The user interface may contain, communicate, generate, obtain, and/or provide program component, system, user, and/or data communications, requests, and/or responses.

Web Browser

A Web browser component 718 is a stored program component that is executed by a CPU. The Web browser may be a conventional hypertext viewing application such as Microsoft Internet Explorer or Netscape Navigator. Secure Web browsing may be supplied with 128 bit (or greater) encryption by way of HTTPS, SSL, and/or the like. Web browsers allowing for the execution of program components through facilities such as ActiveX, AJAX, (D)HTML, FLASH, Java, JavaScript, web browser plug-in APIs (e.g., FireFox, Safari Plug-in, and/or the like APIs), and/or the like. Web browsers and like information access tools may be integrated into PDAs, cellular telephones, and/or other mobile devices. A Web browser may communicate to and/or with other components in a component collection, including itself, and/or facilities of the like. Most frequently, the Web browser communicates with information servers, operating systems, integrated program components (e.g., plug-ins), and/or the like; e.g., it may contain, communicate, generate, obtain, and/or provide program component, system, user, and/or data communications, requests, and/or responses. Of course, in place of a Web browser and information server, a combined application may be developed to perform similar functions of both. The combined application would similarly affect the obtaining and the provision of information to users, user agents, and/or the like from the DIGI™ enabled nodes. The combined application may be nugatory on systems employing standard Web browsers.

Mail Server

A mail server component 721 is a stored program component that is executed by a CPU 703. The mail server may be a conventional Internet mail server such as, but not limited to sendmail, Microsoft Exchange, and/or the like. The mail server may allow for the execution of program components through facilities such as ASP, ActiveX, (ANSI) (Objective−) C (++), C# and/or .NET, CGI scripts, Java, JavaScript, PERL, PHP, pipes, Python, WebObjects, and/or the like. The mail server may support communications protocols such as, but not limited to: Internet message access protocol (IMAP), Messaging Application Programming Interface (MAPI)/Microsoft Exchange, post office protocol (POP3), simple mail transfer protocol (SMTP), and/or the like. The mail server can route, forward, and process incoming and outgoing mail messages that have been sent, relayed and/or otherwise traversing through and/or to the DIGI™.

Access to the DIGI™ mail may be achieved through a number of APIs offered by the individual Web server components and/or the operating system.

Also, a mail server may contain, communicate, generate, obtain, and/or provide program component, system, user, and/or data communications, requests, information, and/or responses.

Mail Client

A mail client component 722 is a stored program component that is executed by a CPU 703. The mail client may be a conventional mail viewing application such as Apple Mail, Microsoft Entourage, Microsoft Outlook, Microsoft Outlook Express, Mozilla, Thunderbird, and/or the like. Mail clients may support a number of transfer protocols, such as: IMAP, Microsoft Exchange, POP3, SMTP, and/or the like. A mail client may communicate to and/or with other components in a component collection, including itself, and/or facilities of the like. Most frequently, the mail client communicates with mail servers, operating systems, other mail clients, and/or the like; e.g., it may contain, communicate, generate, obtain, and/or provide program component, system, user, and/or data communications, requests, information, and/or responses. Generally, the mail client provides a facility to compose and transmit electronic mail messages.

Cryptographic Server

A cryptographic server component 720 is a stored program component that is executed by a CPU 703, cryptographic processor 726, cryptographic processor interface 727, cryptographic processor device 728, and/or the like. Cryptographic processor interfaces will allow for expedition of encryption and/or decryption requests by the cryptographic component; however, the cryptographic component, alternatively, may run on a conventional CPU. The cryptographic component allows for the encryption and/or decryption of provided data. The cryptographic component allows for both symmetric and asymmetric (e.g., Pretty Good Protection (PGP)) encryption and/or decryption. The cryptographic component may employ cryptographic techniques such as, but not limited to: digital certificates (e.g., X.509 authentication framework), digital signatures, dual signatures, enveloping, password access protection, public key management, and/or the like. The cryptographic component will facilitate numerous (encryption and/or decryption) security protocols such as, but not limited to: checksum, Data Encryption Standard (DES), Elliptical Curve Encryption (ECC), International Data Encryption Algorithm (IDEA), Message Digest 5 (MD5, which is a one way hash function), passwords, Rivest Cipher (RC5), Rijndael, RSA (which is an Internet encryption and authentication system that uses an algorithm developed in 1977 by Ron Rivest, Adi Shamir, and Leonard Adleman), Secure Hash Algorithm (SHA), Secure Socket Layer (SSL), Secure Hypertext Transfer Protocol (HTTPS), and/or the like. Employing such encryption security protocols, the DIGI™ may encrypt all incoming and/or outgoing communications and may serve as node within a virtual private network (VPN) with a wider communications network. The cryptographic component facilitates the process of “security authorization” whereby access to a resource is inhibited by a security protocol wherein the cryptographic component effects authorized access to the secured resource. In addition, the cryptographic component may provide unique identifiers of content, e.g., employing and MD5 hash to obtain a unique signature for a digital audio file. A cryptographic component may communicate to and/or with other components in a component collection, including itself, and/or facilities of the like. The cryptographic component supports encryption schemes allowing for the secure transmission of information across a communications network to enable the DIGI™ component to engage in secure transactions if so desired. The cryptographic component facilitates the secure accessing of resources on the DIGI™ and facilitates the access of secured resources on remote systems; i.e., it may act as a client and/or server of secured resources. Most frequently, the cryptographic component communicates with information servers, operating systems, other program components, and/or the like. The cryptographic component may contain, communicate, generate, obtain, and/or provide program component, system, user, and/or data communications, requests, and/or responses.

The DIGI™ Database

The DIGI™ database component 719 may be embodied in a database and its stored data. The database is a stored program component, which is executed by the CPU; the stored program component portion configuring the CPU to process the stored data. The database may be a conventional, fault tolerant, relational, scalable, secure database such as Oracle or Sybase. Relational databases are an extension of a flat file. Relational databases consist of a series of related tables. The tables are interconnected via a key field. Use of the key field allows the combination of the tables by indexing against the key field; i.e., the key fields act as dimensional pivot points for combining information from various tables. Relationships generally identify links maintained between tables by matching primary keys. Primary keys represent fields that uniquely identify the rows of a table in a relational database. More precisely, they uniquely identify rows of a table on the “one” side of a one-to-many relationship.

Alternatively, the DIGI™ database may be implemented using various standard data-structures, such as an array, hash, (linked) list, struct, structured text file (e.g., XML), table, and/or the like. Such data-structures may be stored in memory and/or in (structured) files. In another alternative, an object-oriented database may be used, such as Frontier, ObjectStore, Poet, Zope, and/or the like. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the DIGI™ database is implemented as a data-structure, the use of the DIGI™ database 719 may be integrated into another component such as the DIGI™ component 735. Also, the database may be implemented as a mix of data structures, objects, and relational structures. Databases may be consolidated and/or distributed in countless variations through standard data processing techniques. Portions of databases, e.g., tables, may be exported and/or imported and thus decentralized and/or integrated.

In one embodiment, the database component 719 includes several tables 719a-n. A Users (e.g., operators and physicians) table 719a may include fields such as, but not limited to: user_id, ssn, dob, first_name, last_name, age, state, address_firstline, address_secondline, zipcode, devices_list, contact_info, contact_type, alt_contact_info, alt_contact_type, and/or the like to refer to any type of enterable data or selections discussed herein. The Users table may support and/or track multiple entity accounts. A Clients table 719b may include fields such as, but not limited to: user_id, client_id, client_ip, client_type, client_model, operating_system, os_version, app_installed_flag, and/or the like. An Apps table 719c may include fields such as, but not limited to: app_ID, app_name, app_type, OS_compatibilities_list, version, timestamp, developer_ID, and/or the like. A blood pressure parameters table 719d including, for example, measured blood pressures and other senser outputs, such as measured_pressure, time_stamp, as well other figures of merit that can be measured using embodiments in accordance with the present disclosure, including but not limited to those specifically addressed herein above.

In one embodiment, user programs may contain various user interface primitives, which may serve to update the DIGI™ platform. Also, various accounts may require custom database tables depending upon the environments and the types of clients the DIGI™ system may need to serve. It should be noted that any unique fields may be designated as a key field throughout. In an alternative embodiment, these tables have been decentralized into their own databases and their respective database controllers (i.e., individual database controllers for each of the above tables). Employing standard data processing techniques, one may further distribute the databases over several computer systemizations and/or storage devices. Similarly, configurations of the decentralized database controllers may be varied by consolidating and/or distributing the various database components 719a-n. The DIGI™ system may be configured to keep track of various settings, inputs, and parameters via database controllers.

The DIGI™ database may communicate to and/or with other components in a component collection, including itself, and/or facilities of the like. Most frequently, the DIGI™ database communicates with the DIGI™ component, other program components, and/or the like. The database may contain, retain, and provide information regarding other nodes and data.

The DIGI™ Components

The DIGI™ component 735 is a stored program component that is executed by a CPU. In one embodiment, the DIGI™ component incorporates any and/or all combinations of the aspects of the DIGI™ systems discussed in the previous figures. As such, the DIGI™ component affects accessing, obtaining and the provision of information, services, transactions, and/or the like across various communications networks.

The DIGI™ component may transform data collected by the device 100 or other input signals received, e.g., from a mobile device, into commands for operating the device 100.

The DIGI™ component enabling access of information between nodes may be developed by employing standard development tools and languages such as, but not limited to: Apache components, Assembly, ActiveX, binary executables, (ANSI) (Objective-) C (++), C# and/or .NET, database adapters, CGI scripts, Java, JavaScript, mapping tools, procedural and object oriented development tools, PERL, PHP, Python, shell scripts, SQL commands, web application server extensions, web development environments and libraries (e.g., Microsoft's ActiveX; Adobe AIR, FLEX & FLASH; AJAX; (D)HTML; Dojo, Java; JavaScript; jQuery(UI); MooTools; Prototype; script.aculo.us; Simple Object Access Protocol (SOAP); SWFObject; Yahoo! User Interface; and/or the like), WebObjects, and/or the like. In one embodiment, the DIGI™ server employs a cryptographic server to encrypt and decrypt communications. The DIGI™ component may communicate to and/or with other components in a component collection, including itself, and/or facilities of the like. Most frequently, the DIGI™ component communicates with the DIGI™ database, operating systems, other program components, and/or the like. The DIGI™ may contain, communicate, generate, obtain, and/or provide program component, system, user, and/or data communications, requests, and/or responses.

### Distributed DIGI™ Embodiments

The structure and/or operation of any of the DIGI™ node controller components may be combined, consolidated, and/or distributed in any number of ways to facilitate development and/or deployment. Similarly, the component collection may be combined in any number of ways to facilitate deployment and/or development. To accomplish this, one may integrate the components into a common code base or in a facility that can dynamically load the components on demand in an integrated fashion.

The component collection may be consolidated and/or distributed in countless variations through standard data processing and/or development techniques. Multiple instances of any one of the program components in the program component collection may be instantiated on a single node, and/or across numerous nodes to improve performance through load-balancing and/or data-processing techniques. Furthermore, single instances may also be distributed across multiple controllers and/or storage devices; e.g., databases. All program component instances and controllers working in concert may do so through standard data processing communication techniques.

The configuration of the DIGI™ controller will depend on the context of system deployment. Factors such as, but not limited to, the budget, capacity, location, and/or use of the underlying hardware resources may affect deployment requirements and configuration. Regardless of if the configuration results in more consolidated and/or integrated program components, results in a more distributed series of program components, and/or results in some combination between a consolidated and distributed configuration, data may be communicated, obtained, and/or provided. Instances of components consolidated into a common code base from the program component collection may communicate, obtain, and/or provide data. This may be accomplished through intra-application data processing communication techniques such as, but not limited to: data referencing (e.g., pointers), internal messaging, object instance variable communication, shared memory space, variable passing, and/or the like.

If component collection components are discrete, separate, and/or external to one another, then communicating, obtaining, and/or providing data with and/or to other component components may be accomplished through inter-application data processing communication techniques such as, but not limited to: Application Program Interfaces (API) information passage; (distributed) Component Object Model ((D)COM), (Distributed) Object Linking and Embedding ((D)OLE), and/or the like), Common Object Request Broker Architecture (CORBA), Jini local and remote application program interfaces, JavaScript Object Notation (JSON), Remote Method Invocation (RMI), SOAP, process pipes, shared files, and/or the like. Messages sent between discrete component components for inter-application communication or within memory spaces of a singular component for intra-application communication may be facilitated through the creation and parsing of a grammar A grammar may be developed by using development tools such as lex, yacc, XML, and/or the like, which allow for grammar generation and parsing capabilities, which in turn may form the basis of communication messages within and between components.

For example, a grammar may be arranged to recognize the tokens of an HTTP post command, e.g.:
w3c -post http:// . . . Value1

where Value1 is discerned as being a parameter because “http://” is part of the grammar syntax, and what follows is considered part of the post value. Similarly, with such a grammar, a variable “Value1” may be inserted into an “http://” post command and then sent. The grammar syntax itself may be presented as structured data that is interpreted and/or otherwise used to generate the parsing mechanism (e.g., a syntax description text file as processed by lex, yacc, etc.). Also, once the parsing mechanism is generated and/or instantiated, it itself may process and/or parse structured data such as, but not limited to: character (e.g., tab) delineated text, HTML, structured text streams, XML, and/or the like structured data. In another embodiment, inter-application data processing protocols themselves may have integrated and/or readily available parsers (e.g., JSON, SOAP, and/or like parsers) that may be employed to parse (e.g., communications) data. Further, the parsing grammar may be used beyond message parsing, but may also be used to parse: databases, data collections, data stores, structured data, and/or the like. Again, the desired configuration will depend upon the context, environment, and requirements of system deployment.

For example, in some implementations, the DIGI™ controller may be executing a PHP script implementing a Secure Sockets Layer (“SSL”) socket server via the information server, which listens to incoming communications on a server port to which a client may send data, e.g., data encoded in JSON format. Upon identifying an incoming communication, the PHP script may read the incoming message from the client device, parse the received JSON-encoded text data to extract information from the JSON-encoded text data into PHP script variables, and store the data (e.g., client identifying information, etc.) and/or extracted information in a relational database accessible using the Structured Query Language (“SQL”). An exemplary listing, written substantially in the form of PHP/SQL commands, to accept JSON-encoded input data from a client device via a SSL connection, parse the data to extract variables, and store the data to a database, is provided below:

```
<?PHP
header(′Content-Type: text/plain′);
// set ip address and port to listen to for incoming data
$address = ‘192.168.0.100’;
$port = 255;
// create a server-side SSL socket, listen for/accept incoming
communication
$sock = socket_create(AF_INET, SOCK_STREAM, 0);
socket_bind($sock, $address, $port) or die(‘Could not bind to address’);
socket_listen($sock);
$client = socket_accept($sock);
// read input data from client device in 1024 byte blocks until end of
message
do {
  $input = “”;
  $input = socket_read($client, 1024);
  $data .= $input;
} while($input != “”);
// parse data to extract variables
$obj = json_decode($data, true);
// store input data in a database
mysql_connect(″201.408.185.132″,$DBserver,$password); // access
database server
mysql_select(″CLIENT_DB.SQL″); // select database to append
mysql_query(“INSERT INTO UserTable (transmission)
VALUES ($data)”); // add data to UserTable table in a CLIENT database
mysql_close(″CLIENT_DB.SQL″); // close connection to database
?>
```

Also, the following resources may be used to provide example embodiments regarding SOAP parser implementation:
http://www.xav.com/perl/site/lib/SOAP/Parser.html
http://publib.boulder.ibm.com/infocenter/tivihelp/v2r1/index.jsp?topic=/com.ibm.IBMDI.doc/referenceguide295.htm

and other parser implementations:
http://publib.boulder.ibm.com/infocenter/tivihelp/v2r1/index.jsp?topic=/com.ibm.IBMDI.doc/referenceguide259.htm

all of which are hereby expressly incorporated by reference.

In order to address various issues and advance the art, the entirety of this application (including the Cover Page, Title, Headings, Field, Background, Summary, Brief Description of the Drawings, Detailed Description, Claims, Abstract, Figures, Appendices and/or otherwise) shows by way of illustration various embodiments in which the claimed inventions may be practiced. The advantages and features of the application are of a representative sample of embodiments only, and are not exhaustive and/or exclusive. They are presented only to assist in understanding and teach the claimed principles. It should be understood that they are not representative of all disclosed embodiments. As such, certain aspects of the disclosure have not been discussed herein. That alternate embodiments may not have been presented for a specific portion of the invention or that further undescribed alternate embodiments may be available for a portion is not to be considered a disclaimer of those alternate embodiments. It will be appreciated that many of those undescribed embodiments incorporate the same principles of the invention and others are equivalent. Thus, it is to be understood that other embodiments may be utilized and functional, logical, organizational, structural and/or topological modifications may be made without departing from the scope and/or spirit of the disclosure. As such, all examples and/or embodiments are deemed to be non-limiting throughout this disclosure. Also, no inference should be drawn regarding those embodiments discussed herein relative to those not discussed herein other than it is as such for purposes of reducing space and repetition. For instance, it is to be understood that the logical and/or topological structure of any combination of any program components (a component collection), other components and/or any present feature sets as described in the figures and/or throughout are not limited to a fixed operating order and/or arrangement, but rather, any disclosed order is exemplary and all equivalents, regardless of order, are contemplated by the disclosure. Furthermore, it is to be understood that such features are not limited to serial execution, but rather, any number of threads, processes, services, servers, and/or the like that may execute asynchronously, concurrently, in parallel, simultaneously, synchronously, and/or the like are contemplated by the disclosure. As such, some of these features may be mutually contradictory, in that they cannot be simultaneously present in a single embodiment. Similarly, some features are applicable to one aspect of the invention, and inapplicable to others. In addition, the disclosure includes other inventions not presently claimed. Applicant reserves all rights in those presently unclaimed inventions including the right to claim such inventions, file additional applications, continuations, continuations in part, divisions, and/or the like thereof. As such, it should be understood that advantages, embodiments, examples, functional, features, logical, organizational, structural, topological, and/or other aspects of the disclosure are not to be considered limitations on the disclosure as defined by the claims or limitations on equivalents to the claims. It is to be understood that, depending on the particular needs and/or characteristics of a DIGI™ individual and/or enterprise user, database configuration and/or relational model, data type, data transmission and/or network framework, syntax structure, and/or the like, various embodiments of the DIGI™ may be implemented that enable a great deal of flexibility and customization.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Descriptions herein of circuitry and method steps and computer programs represent conceptual embodiments of illustrative circuitry and software embodying the principles of the disclosed embodiments. Thus the functions of the various elements shown and described herein may be provided through the use of dedicated hardware as well as hardware capable of executing software in association with appropriate software as set forth herein.

Terms to exemplify orientation, such as upper/lower, left/right, top/bottom and above/below, may be used herein to refer to relative positions of elements as shown in the figures. It should be understood that the terminology is used for notational convenience only and that in actual use the disclosed structures may be oriented different from the orientation shown in the figures. Thus, the terms should not be construed in a limiting manner.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein.

Similarly, it will be appreciated that the system and process flows described herein represent various processes which may be substantially represented in computer-readable media and so executed by a computer or processor, whether or not such computer or processor is explicitly shown. Moreover, the various processes can be understood as representing not only processing and/or other functions but, alternatively, as blocks of program code that carry out such processing or functions.

As examples, the Specification describes and/or illustrates aspects useful for implementing the claimed disclosure by way of various circuits or circuitry which may be illustrated as or using terms such as blocks, modules, device, system, unit, controller, and/or other circuit-type depictions. Such circuits or circuitry are used together with other elements to exemplify how certain embodiments may be carried out in the form or structures, steps, functions, operations, activities, etc. In certain embodiments, such illustrated items represent one or more computer circuitry (e.g., microcomputer or other CPU) which is understood to include memory circuitry that stores code (program to be executed as a set/sets of instructions) for performing an algorithm. The specification may also make reference to an adjective that does not connote any attribute of the structure (“first [type of structure]” and “second [type of structure]”) in which case the adjective is merely used for English-language antecedence to differentiate one such similarly-named structure from another similarly-named structure (e.g., “first circuit configured to convert . . . ” is interpreted as “circuit configured to convert . . . ”). On the other hand, specification may make reference to an adjective that is intended to connote an attribute of the structure (e.g., monitor server), in which case the adjective (e.g., monitor) modifies to refer to at least a portion of the named structure (e.g., server) is configured to have/perform that attribute (e.g., monitor server refers to at least a portion of a server that includes/performs the attribute of monitoring.

Some portions of the above description present the techniques described herein in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

The methods, systems, computer programs and mobile devices of the present disclosure, as described above and shown in the drawings, among other things, provide for improved methods, systems and machine readable programs for carrying out blood pressure measurement. It will be apparent to those skilled in the art that various modifications and variations can be made in the devices, methods, software programs and mobile devices of the present disclosure without departing from the spirit or scope of the disclosure. Thus, it is intended that the present disclosure include modifications and variations that are within the scope of the subject disclosure and equivalents.

What is claimed is:

1. A device for measuring blood pressure including detection, processing and display circuitry, the device comprising:
a pc board including circuitry;
a measuring surface to receive a user's anatomic region of interest to detect blood pressure;
a first circuit configured and arranged to detect, by a first sensor, a pressure or force applied to the measuring surface;
a second circuit configured and arranged to detect, by a second sensor, measured blood volume oscillations in an anatomical region of interest while force is being applied to the sensor by the anatomical region of interest; and
processing circuitry configured and arranged to estimate a blood pressure value for the user from the measured pressure or force and the measured blood volume oscillations.

2. The device of claim 1, wherein the circuitry is configured and arranged to place a visual indicator on the measuring surface of the device, wherein the visual indicator is arranged in relation to the first and second sensors.

3. The device of claim 2, wherein at least one of the first sensor and second sensor is integrated into the measuring surface of the device.

4. The device of claim 1, further comprising a compliant deflectable mass disposed adjacent to the measuring surface of the device.

5. The device of claim 4, wherein the deflectable mass includes a gel material.

6. The device of claim 5, wherein the gel material is covered by a membrane that at least partially defines the measuring surface.

7. The device of claim 5, wherein the first sensor is at least partially embedded in the compliant deflectable mass.

8. The device of claim 4, wherein the deflectable mass includes a fluid surrounded by a bladder, and further wherein the bladder at least partially defines the measuring surface.

9. The device of claim 1, further comprising a housing to contain the pc board.

10. The device of claim 9, wherein the housing includes a shell that at least partially defines the measuring surface.

11. The device of claim 10, wherein the shell defines a first opening therethrough within a line of sight of the second sensor.

12. The device of claim 11, wherein the housing defines a second opening to permit a line of sight from the first opening through the second opening to a detector of a further device.

13. The device of claim 1, further comprising at least one LED to direct light into the anatomical region of interest.

14. The device of claim 1, wherein the second sensor is disposed on a movable platform displaceable from a first position from a second position, wherein movement from the first position to the second position is resisted by a resilient element.

15. The device of claim 1, wherein the measuring surface defines a cavity therein to accommodate tissue to permit a pooling of blood within the tissue.

16. The device of claim 1, wherein at least one of the sensors includes a photoplethysmography (PPG) sensor and a pressure sensor.

17. The device of claim 16, wherein the PPG sensor is an array of PPG sensors including a plurality of PPG sensors and further wherein the device is configured and arranged to choose the sensor that yields a largest amplitude oscillogram.

18. A processor-readable tangible non-transient medium storing a computer program for operating an arrangement of processing circuitry including a device for measuring blood pressure communicatively coupled to at least one processing circuit according to any preceding claim, the computer program comprising:
instructions configured to measure, by the sensor, a measured pressure applied to the sensor;
instructions configured to measure, by the sensor, measured blood volume oscillations in the anatomical region of interest while pressure is being applied to the sensor by the anatomical region of interest; and
instructions configured to estimate, by the processing circuitry of the device, a blood pressure value for the user from the measured pressure and the measured blood volume oscillations.

19. The processor-readable tangible non-transient medium of claim 18, wherein the computer program further includes instructions configured to place a visual indicator on an exterior surface of the device, wherein the visual indicator is arranged in relation to the sensor and the sensor is integrated into the exterior surface of the device.

20. The processor-readable tangible non-transient medium of claim 18, wherein the computer program further includes instructions configured to construct two or more oscillograms for the user from the measured pressure and the measured blood volume oscillations, wherein each oscillogram in the two or more oscillograms is constructed from measurements taken at different placements of the anatomy of interest in relation to the sensor.
